SEQUENCE LISTING (121 sequences)

SEQ ID NO: 1 - DNA, 661 bp, human
  misc_feature (533)..(533): n = any nucleotide
gtaggtggggaaggggtgtcaggtgggtactgcagatgggctctaggacctcggccttcaagttgtgtctgcccgcctcttgctactgtcttggatattttaaagtccttttgacgttgttctgatttctgggcaggggacagagtaagtgtgtatttgctctgagactgttaatttggtatttccatcccaagttacagggaagacctcaggctgcaggttcctagctccgggctgaggtggcttgtggaggcagacagctgttgtctggaagtgcagagggctgggggctggccaggctgttactgagttcagaataggaggaaagagtgtgtagcaaagtcggcgctccttggccactgccagcattcagagttgtcttgtttgccttgccttaaacgttgccttcctggacgcctacaaagtcaggttgtaaccgctggccactgctgtgctcactggcagcccctgatttacgtgaggacctcaagtgtgtgttgggcagaattccccagcgcttcccgtacaccccnccacccccagtgcagcatcgctcggtgcgtggctggtggactggaggagtgtgcgtgccggcagcactgccaggcacgtgcctaatgctctggccctgtgtgtttgtgttttcttcccgatttctgag

SEQ ID NO: 2 - DNA, 20 bp, human
agtgcagagggctgggggct

SEQ ID NO: 3 - DNA, 20 bp, human
agtgcagagggctgggggct

SEQ ID NO: 4 - DNA, 20 bp, human
ttgtctggaagtgcagaggg

SEQ ID NO: 5 - DNA, 20 bp, human
ttgtctggaagtgcagaggg

SEQ ID NO: 6 - DNA, 20 bp, human
tggctggtggactggaggag

SEQ ID NO: 7 - DNA, 20 bp, human
tggctggtggactggaggag

SEQ ID NO: 8 - DNA, 20 bp, human
gcttgtggaggcagacagct

SEQ ID NO: 9 - DNA, 20 bp, human
gcttgtggaggcagacagct

SEQ ID NO: 10 - DNA, 20 bp, human
agtgcagagggctgggggct

SEQ ID NO: 11 - DNA, 20 bp, human
agtgcagagggctgggggct

SEQ ID NO: 12 - DNA, 19 bp, human
tttgctctgagactgttaa

SEQ ID NO: 13 - DNA, 19 bp, human
tttgctctgagactgttaa

SEQ ID NO: 14 - DNA, 19 bp, human
agggctgggggctggccag

SEQ ID NO: 15 - DNA, 19 bp, human
agggctgggggctggccag

SEQ ID NO: 16 - DNA, 19 bp, human
gttgttctgatttctgggc

SEQ ID NO: 17 - DNA, 19 bp, human
gttgttctgatttctgggc

SEQ ID NO: 18 - DNA, 19 bp, human
tgtgtgtttgtgttttctt

SEQ ID NO: 19 - DNA, 19 bp, human
tgtgtgtttgtgttttctt

SEQ ID NO: 20 - DNA, 19 bp, human
agagggctgggggctggcc

SEQ ID NO: 21 - DNA, 19 bp, human
agagggctgggggctggcc

SEQ ID NO: 22 - DNA, 23 bp, human
gccctgtgtgtttgtgttttctt

SEQ ID NO: 23 - DNA, 23 bp, human
gccctgtgtgtttgtcttttctt

SEQ ID NO: 24 - DNA, 19 bp, human
agagggctgggggctggcc

SEQ ID NO: 25 - DNA, 19 bp, human
agagggctgggggctggcc

SEQ ID NO: 26 - DNA, 19 bp, human
tgtgtgtttgtgttttctt

SEQ ID NO: 27 - DNA, 19 bp, human
tgtgtgtttgtgttttctt

SEQ ID NO: 28 - DNA, 19 bp, human
agcccctgatttacgtgag

SEQ ID NO: 29 - DNA, 19 bp, human
agcccctgatttacgtgag

SEQ ID NO: 30 - DNA, 19 bp, human
gtgtgtttgtgttttcttc

SEQ ID NO: 31 - DNA, 19 bp, human
gtgtgtttgtgttttcttc

SEQ ID NO: 32 - DNA, 19 bp, human
gcagagggctgggggctgg

SEQ ID NO: 33 - DNA, 19 bp, human
gcagagggctgggggctgg

SEQ ID NO: 34 - DNA, 19 bp, human
gccttcctggacgcctaca

SEQ ID NO: 35 - DNA, 19 bp, human
gccttcctggacgcctaca

SEQ ID NO: 36 - DNA, 19 bp, human
tttgctctgagactgttaa

SEQ ID NO: 37 - DNA, 19 bp, human
tttgctctgagactgttaa

SEQ ID NO: 38 - DNA, 19 bp, human
ttaaacgttgccttcctgg

SEQ ID NO: 39 - DNA, 19 bp, human
ttaaacgttgccttcctgg

SEQ ID NO: 40 - DNA, 27 bp, human
tttctgggcaggggacagagtaagtgt

SEQ ID NO: 41 - DNA, 27 bp, human
tttctgggtaggggacagagtatgtgt

SEQ ID NO: 42 - DNA, 19 bp, human
gaattccccagcgcttccc

SEQ ID NO: 43 - DNA, 19 bp, human
gaattccccagcgcttccc

SEQ ID NO: 44 - DNA, 295 bp, human
gtaagtgcccttccgggagctcacacccgctctctgtctcccctgtccttcctctgcttcattttttcctggactctgaccgatgtttgcgttagagtatgtttgaacgtggggtcgattgggaaggattaagccttggtgctgaggctggatattgcaggaggatacagggtgaatggagccggcggggcggggcgggccgggctgctgtgccgtggctgctgttgtgctgacaccctctttcctagagaaacagcctcttattcacaaccagctgatttgaaatttcctgcag

SEQ ID NO: 45 - DNA, 22 bp, human
ggcggggcggggcgggccgggc

SEQ ID NO: 46 - DNA, 22 bp, human
ggcggggcggggcgggccgggc

SEQ ID NO: 47 - DNA, 22 bp, human
ggcggggcggggcgggccgggc

SEQ ID NO: 48 - DNA, 22 bp, human
ggcggggcggggcgggccgggc

SEQ ID NO: 49 - DNA, 21 bp, human
gcggggcggggcgggccgggc

SEQ ID NO: 50 - DNA, 21 bp, human
gcggggcggggcgggccgggc

SEQ ID NO: 51 - DNA, 21 bp, human
gcggggcggggcgggccgggc

SEQ ID NO: 52 - DNA, 21 bp, human
gcggggcggggcgggccgggc

SEQ ID NO: 53 - DNA, 658 bp, human
gtatgtaccgtgctgggaccacttccccaggtgccttccccacccagccaggtctgtagttttgaaagtcttgtatagctttttccttggtttaaaagcaataaatgcccactggagataaattagaaaatatggaagaaagctataaaaaagaaactaaaaaaatctcttgtaattccaccactcaaatataactttttttcttaaaaaattttttttctcttacttagagacaggcagggtctggctctgtcccccaggctggagtgcagtggtgccatcatagctcactgcagcctcaacctcttgggctcaaggcattctctcgcctcagcctcctgagcagctgggactgcaggcatgagccatggttcctgggcattttctcttgatattttgatgaagcagcctctttgtccccaggtcatagctgcttaagacactatgtacagagatcttagttgaatgagacaagtgacttctggctgtgccctgcagataggccttgggtgcagccatggtttgtagattcccctggagaaatccaagcaacacacatgtatttggtactcactaagtgcctacagaaccaaaccgaaactgggccgcactggggaggagatcaccgtggagaccggagggcgcactcacggagagt

SEQ ID NO: 54 - DNA, 60 bp, human
cagggtctggctctgtcccccaggctggagtgcagtggtgccatcatagctcactgcagc

SEQ ID NO: 55 - DNA, 60 bp, human
cagggtcttgctctgttgcccaggctggggtgcagtggcgcaatcatggctcactgcagc

SEQ ID NO: 56 - DNA, 60 bp, human
ctcaacctcttgggctcaaggcattctctcgcctcagcctcctgagcagctgggactgca

SEQ ID NO: 57 - DNA, 60 bp, human
ctcaacctcctgggctcaagccatcctcccgcctcagcctcctgagcagctgggactaca

SEQ ID NO: 58 - DNA, 60 bp, human
gctctgtcccccaggctggagtgcagtggtgccatcatagctcactgcagcctcaacctc

SEQ ID NO: 59 - DNA, 60 bp, human
gctctgtcacccaggctggagtgtagtggtgcaatcagagctcactgcagcctccaactc

SEQ ID NO: 60 - DNA, 47 bp, human
ttgggctcaaggcattctctcgcctcagcctcctgagcagctgggac

SEQ ID NO: 61 - DNA, 47 bp, human
ctgggctcaagcaatcctcccacctcagcctcctgagtagctaggac

SEQ ID NO: 62 - DNA, 60 bp, human
ctctgtcccccaggctggagtgcagtggtgccatcatagctcactgcagcctcaacctct

SEQ ID NO: 63 - DNA, 60 bp, human
ctctgtcacccaggctggagtgcagtggtgcgatcttggctcactgcaacctccgcctcc

SEQ ID NO: 64 - DNA, 60 bp, human
tgggctcaaggcattctctcgcctcagcctcctgagcagctgggactgcaggcatgagcc

SEQ ID NO: 65 - DNA, 60 bp, human
tgggttcaagtgattctcctgcctcagcctcccgagtagctgggactacaggcgtgtgcc

SEQ ID NO: 66 - DNA, 60 bp, human
gtggggacaaacagaaagacacaaggaacaattagaggctctccatagcaatgtcagaga

SEQ ID NO: 67 - DNA, 60 bp, human; identical to SEQ ID NO: 66

SEQ ID NO: 68 - DNA, 60 bp, human
tagggcagagcggatggtggtgacaacgctctgacaaacgttactattgaacgagagtca

SEQ ID NO: 69 - DNA, 60 bp, human; identical to SEQ ID NO: 68

SEQ ID NO: 70 - DNA, 60 bp, human; identical to SEQ ID NO: 54

SEQ ID NO: 71 - DNA, 60 bp, human
cagggtcttgctctgtcacccaggctggagttcagtggtgcaatcatagctcactgcagc

SEQ ID NO: 72 - DNA, 60 bp, human; identical to SEQ ID NO: 56

SEQ ID NO: 73 - DNA, 60 bp, human
ctcaaactcctgggctcaagcaatcctcccacctcagcctcctgagtagctgggactgca

SEQ ID NO: 74 - DNA, 60 bp, human
ctgtcccccaggctggagtgcagtggtgccatcatagctcactgcagcctcaacctcttg

SEQ ID NO: 75 - DNA, 60 bp, human
ctgtcacccaggctggagtgcagtggcgccatcatggctcactgcagcctcaacctcctg

SEQ ID NO: 76 - DNA, 59 bp, human
ggctcaaggcattctctcgcctcagcctcctgagcagctgggactgcaggcatgagcca

SEQ ID NO: 77 - DNA, 59 bp, human
ggctcaagccatcctaccacctcagcctcctgagtagctggaactacaggcatgggcca

SEQ ID NO: 78 - DNA, 60 bp, human
ggtctggctctgtcccccaggctggagtgcagtggtgccatcatagctcactgcagcctc

SEQ ID NO: 79 - DNA, 60 bp, human
ggtctcgctctgtcactcaggctggagtgcagtggtgccatcacagctcactgcagcctc

SEQ ID NO: 80 - DNA, 44 bp, human
aacctcttgggctcaaggcattctctcgcctcagcctcctgagc

SEQ ID NO: 81 - DNA, 44 bp, human
aaattcttgggctcaagccatcctctcacctcagcctcctgagc

SEQ ID NO: 82 - DNA, 16 bp, Saccharomyces cerevisiae
cttattttttcattat

SEQ ID NO: 83 - DNA, 16 bp, Saccharomyces cerevisiae
tttttcattatgaaaa

SEQ ID NO: 84 - DNA, 16 bp, Saccharomyces cerevisiae
aaaatatttgttagta

SEQ ID NO: 85 - DNA, 16 bp, Saccharomyces cerevisiae
ctgctgtagaggttct

SEQ ID NO: 86 - DNA, 18 bp, Saccharomyces cerevisiae
ctaataatttggaaagga

SEQ ID NO: 87 - DNA, 16 bp, Saccharomyces cerevisiae
ataacatttttaaaac

SEQ ID NO: 88 - DNA, 16 bp, Saccharomyces cerevisiae
ggttctttcccccttt

SEQ ID NO: 89 - DNA, 17 bp, Saccharomyces cerevisiae
ctaataatttggaaagg

SEQ ID NO: 90 - DNA, 16 bp, Saccharomyces cerevisiae
aagtggtttttctgga

SEQ ID NO: 91 - DNA, 16 bp, Saccharomyces cerevisiae
tagataataaaagaaa

SEQ ID NO: 92 - DNA, 16 bp, Saccharomyces cerevisiae
ctagataataaaagaa

SEQ ID NO: 93 - DNA, 16 bp, Saccharomyces cerevisiae
gttaagtattttttta

SEQ ID NO: 94 - DNA, 16 bp, Saccharomyces cerevisiae
cctttcaaaacttata

SEQ ID NO: 95 - DNA, 16 bp, Saccharomyces cerevisiae
atttgttagtatatgt

SEQ ID NO: 96 - DNA, 16 bp, Saccharomyces cerevisiae
tctttctttccttctt

SEQ ID NO: 97 - DNA, 16 bp, Saccharomyces cerevisiae
tatgtttttttctttt

SEQ ID NO: 98 - DNA, 16 bp, Saccharomyces cerevisiae
tcttcataaaaaagca

SEQ ID NO: 99 - DNA, 17 bp, Saccharomyces cerevisiae
ttctttttctttctttc

SEQ ID NO: 100 - DNA, 16 bp, Saccharomyces cerevisiae
gtatgtttttttcttt

SEQ ID NO: 101 - DNA, 18 bp, Saccharomyces cerevisiae
ctttttctttctttcctt

SEQ ID NO: 102 - DNA, 17 bp, Saccharomyces cerevisiae
tttttttcttttattct

SEQ ID NO: 103 - DNA, 16 bp, Saccharomyces cerevisiae
ttttattctactttta

SEQ ID NO: 104 - DNA, 17 bp, Saccharomyces cerevisiae
aatttaacgatgagatg

SEQ ID NO: 105 - DNA, 17 bp, Saccharomyces cerevisiae
caaacacagaatcattt

SEQ ID NO: 106 - DNA, 17 bp, Saccharomyces cerevisiae
cgatgagatgagctgtg

SEQ ID NO: 107 - DNA, 16 bp, Saccharomyces cerevisiae
ttttttttgtttttga

SEQ ID NO: 108 - DNA, 16 bp, Saccharomyces cerevisiae
ttaattttttttgaat

SEQ ID NO: 109 - DNA, 17 bp, Saccharomyces cerevisiae
taattttttttgaattt

SEQ ID NO: 110 - DNA, 16 bp, Saccharomyces cerevisiae
ttttttttgaattttt

SEQ ID NO: 111 - DNA, 16 bp, Saccharomyces cerevisiae
tttttttgaatttttt

SEQ ID NO: 112 - DNA, 16 bp, Saccharomyces cerevisiae
agttttaatttttttt

SEQ ID NO: 113 - DNA, 16 bp, Saccharomyces cerevisiae
tttttttttgtttttg

SEQ ID NO: 114 - DNA, 18 bp, Saccharomyces cerevisiae
tttttttgtttttgattt

SEQ ID NO: 115 - DNA, 16 bp, Saccharomyces cerevisiae
ttgaatttttttttgt

SEQ ID NO: 116 - DNA, 16 bp, Saccharomyces cerevisiae
ttttaattttttttga

SEQ ID NO: 117 - DNA, 16 bp, Saccharomyces cerevisiae
aataaattgtactcac

SEQ ID NO: 118 - DNA, 17 bp, Saccharomyces cerevisiae
tttttgaattttttttt

SEQ ID NO: 119 - DNA, 16 bp, Saccharomyces cerevisiae
aaaattcaaaaaaaat

SEQ ID NO: 120 - DNA, 16 bp, Saccharomyces cerevisiae
aaaaaaattcaaaaaa

SEQ ID NO: 121 - DNA, 16 bp, Saccharomyces cerevisiae
tttttttttgttcatg

(19) United States
(12) Patent Application Publication
(10) Pub. No.: US 2004/0265865 A1
(43) Pub. Date: Dec. 30, 2004

(54) Method for identifying effector molecules

(76) Inventors: John Mattick, Indooroopilly (AU); Michael Gagen, Taringa (AU); Stefan Stanley, Highgate Hill (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/804,859

(22) Filed: Mar. 19, 2004

Related U.S. Application Data
(63) Continuation-in-part of application No. PCT/AU02/01286, filed on Sep. 19, 2002.
(60) Provisional application No. 60/324,127, filed on Sep. 19, 2001.

(51) Int. Cl. C12Q 1/68; C07H 21/02

(52) U.S. Cl. 435/6; 536/23.1

(57) ABSTRACT

The present invention relates generally to the field of bioinformatics and its applications to functional genomics and advanced genetic engineering. More particularly, the present invention contemplates a method for identifying effector molecules capable of modulating gene network integration and which facilitate genetic multi-tasking and the regulation of complex suites of programmed responses within, on and between eukaryotic cells. The present invention permits, therefore, the identification of a new generation of proteome and nucleome modulators useful in a range of therapeutic and trait-modifying protocols. The ability to manipulate genetic networks within a cell and within whole organisms also provides a sophisticated genetic engineering approach of introducing new traits and to influencing the genetic architecture and, hence, to enable cell and organismal programming or re-programming. The identification of effector molecules and their target or receiver sites, further enables the development of diagnostic protocols for a range of conditions or physiological or genetic states of an organism useful, for example, in modulating stem cell differentiation, quantitative traits, aging or the development of pathological conditions.

### FIELD OF THE INVENTION

The present invention relates generally to the field of bioinformatics and its applications to functional genomics and advanced genetic engineering. More particularly, the present invention contemplates a method for identifying effector molecules capable of modulating gene network integration and which facilitate genetic multi-tasking and the regulation of complex suites of programmed responses within, on and between eukaryotic cells. The present invention permits, therefore, the identification of a new generation of proteome and nucleome modulators useful in a range of therapeutic and trait-modifying protocols. The ability to manipulate genetic networks within a cell and within whole organisms also provides a sophisticated genetic engineering approach of introducing new traits and to influencing the genetic architecture and, hence, to enable cell and organismal programming or re-programming. The identification of effector molecules and their target or receiver sites, further enables the development of diagnostic protocols for a range of conditions or physiological or genetic states of an organism, for example, in modulating stem cell differentiation, quantitative traits, aging or the development of pathological conditions.

### BACKGROUND OF THE INVENTION

Bibliographic details of references provided in the subject specification are listed at the end of the specification.

Reference to any prior art in this specification is not, and should not be taken as, an acknowledgment or any form of suggestion that this prior art forms part of the common general knowledge in any country.

The current understanding of the relationship between genetic information and biological function is predicated in the one gene-one protein hypothesis and in the classical studies of the lac operon and the “genetic code”, i.e. the triplet code specifying amino acids in protein coding sequences. The concept of DNA as a relatively stable, heritable source of template information for proteins, transduced through a temporary and discrete RNA readout has influenced ideas on the structure of genetic systems. Accordingly, cells and organisms are thought of as being built from a myriad of structural and catalytic proteins, whose expression is generally controlled by other regulatory proteins which bind to DNA. This is a biochemical rather than an informatic perspective, which, apart from local analysis of promoter function, gives little thought to the problem of how complex programs of gene activity in the higher organisms might be integrated and regulated in four dimensions.

Genome sequencing projects have shown that the core proteome sizes of Caenorhabditis elegans and Drosophila melanogaster are of similar size and each only about twice the size of yeast and some bacteria, despite these animals' every appearance of possessing more than twice the complexity of microorganisms (Chervitz et al., Science 282: 2022-2028, 1998; Rubin et al., Science 287: 2204-2215, 2000), leading to the conclusion that “the evolution of additional complex attributes is essentially an organizational one; a matter of novel interactions that derive from the temporal and spatial segregation of fairly similar components” (Rubin et al., Science 287: 2204-2215, 2000). This conclusion is reinforced by the finding that the human genome has only about 30,000 protein coding genes (Roest Crollius et al., Nature Genet. 25: 235-238, 2000; Consortium, Nature 409: 860-921, 2001; Venter et al., Science 291: 1304-1351, 2001), the vast majority of which are shared in common with the mouse. The increased complexity of the higher eukaryotes is related, at least in part, to the production of different protein isoforms from the same gene by alternative splicing (Croft et al., Nature Genet. 24: 340-341, 2000). However, perhaps the most surprising and yet so far least considered feature of the genomes of the complex organisms, relative to simpler organisms, is the huge increase in the output of non-protein-coding RNA sequences, which have been estimated to account for around 97-98% of all transcriptional output from the human genome (Mattick, EMBO Reports 2: 986-991, 2001) (see below).

The view that phenotypic variation in complex organisms results from the differential use of a set of core components is becoming common (Duboule and Wilkins, Trends. Genet. 14: 54-59, 1998) and includes such concepts as “synexpression groups” (Niehrs and Pollet, Nature 402: 483-487, 1999), “syntagms” of interacting genes (Huang, Int. J. Dev. Biol. 42: 487-494, 1998) and gene cassettes (Jan and Jan, Proc. Natl. Acad. Sci. USA 90: 8305-8307, 1993), the re-use of modules in signaling pathways (Pawson, Nature 373: 573-580, 1995; Hunter, Cell 100: 113-127, 2000a) and enhanced rates of evolution by varying connections between modular network components (Hartwell et al., Nature 402: C47-52, 1999; Holland Nature 402: C-41-44, 1999). These concepts have been drawn primarily from electrical circuit design and have focussed principally on the modules rather than on the interconnecting control architecture of the system.

Particular network models, which range in size from single regulated circuits (Mestl et al., J. Theor. Biol. 176: 291-300, 1995; Mendoza and Alvarez-Buylla, J. Theor. Biol. 193: 307-319, 1998; Yuh et al., Science 279: 1896-1902, 1998) to complete genomes (Thieffry et al., Bioessays 20: 433-440, 1998) have demonstrated that feedback subnetworks can exhibit computational behaviors including “learned behavior” (Bhalla and Iyengar, Science 283: 381-387, 1999) that switching networks and transcriptional control networks can exhibit dynamical stability (Wolf and Eeckman, J. Theor. Biol. 195: 167-186, 1998; Smolen et al., Am. J. Physiol. 277: C777-790, 1999) and that feedback circuits can implement oscillators governing cell cycles and circadian clocks (Dano et al., Nature 402: 320-322, 1999; Haase and Reed, Nature 401: 394-397, 1999; Shearman et al., Science 288: 1013-1019, 2000). Stochastic noise and time delays allowing feedback, molecular memory and oscillations can be incorporated into such circuit models (Smolen et al., Am. J. Physiol. 277: C777-790, 1999) generating probabilistic phenotypic variation (McAdams and Arkin, Proc. Natl. Acad. Sci. USA 94: 814-819, 1997) and amplification of signals (Hasty t al., Proc. Natl. Acad. Sci. USA 97: 2075-2080, 2000). Some of these models have been verified by synthesizing circuits in cells to feature bistability, oscillations and stochastic destruction of temporal correlations (Becskei and Serrano, Nature 405: 590-593, 2000; Elowitz and Leibler, Nature 403: 335-338, 2000; Gardner et al., Nature 403: 339-342, 2000).

However, such models are unsuited to the analysis of global cellular connectivity and dynamics as they cannot be scaled up to large network sizes, since linear increases in the number of interconnected circuit nodes requires quadratic increases in the number of interconnecting molecules. This leads to an explosive increase in model size which severely constrains numerical simulations using current computing technologies (see e.g. Weng et al., Science 284: 92-96, 1999). A number of alternate approaches have sought to avoid this size explosion by treating sub-networks as active integrated logic components which are interconnected into larger networks (McAdams and Shapiro, Science 269: 650-656, 1995) or by exploiting hierarchically organized control systems to significantly decrease analytical complexity (van der Gugten and Westerhoff, Biosystems 44: 79-106, 1997).

In work leading up to the present invention, the inventors reasoned that biology has solved this problem differentily, and that the types of network control architecture which are used to integrate and multi-task computers and which are used in the brain to coordinate complex activities such as motor coordination and cognition, may also be employed by molecular biological networks to generate phenotypic complexity and variability.

Multi-tasking is employed in every computer where control codes (program instructions) of n bits set the central processing circuit to process one of 2ⁿ different operations. Sequences of control codes (a program) can be internally stored in memory creating a self-contained programmed response network—a computer—as originally defined by von Neumann in 1945 (von Neumann, First Draft of a report on the EDVAC. In: B. Randall, ed. The origins of digital computers: selected papers. Spring, Berlin, 1982). Prior to the arrival of the von Neumann computing architecture, a computer could only be reprogrammed by laborious re-wiring of the central processing unit, while subsequently re-programming simply required loading new control codes into memory. In all computing networks, processing requires not only stored program instructions, but also communication between nodes to synchronize and integrate network activity. The present inventors propose, in accordance with the present invention, that gene networks could exploit similar technology using internal controls based on RNA to multi-task components and sub-networks to generate a wide range of programmed responses, such as in differentiation and development. This system has interesting and perhaps mutually informative analogies with small world networks and dataflow computing.

Existing genetic circuit models, although sophisticated, ignore endogenous controlled multi-tasking and consider each molecular sub-network (involving a few genes for instance) to be sparsely interconnected, and either off or on to express only one dynamical output (see e.g. McAdams and Shapiro, Science 269: 650-656 1995; Bhalla and Iyengar, Science 283: 381-387 1999; Weng et al., Science 284: 92-96 1999). Such models require more complex genetic programs to be built from many sub-networks encoded by exponentially large numbers of genes, a severe constraint, both in theory and in practice. In contrast, multi-tasking via n controls (single molecules suffice) can, in theory, achieve exponential (2ⁿ) multi-tasking of sub-network dynamical outputs, and allow a wide range of programmed responses to be obtained from limited numbers of sub-networks (and genetic coding information). The imbalance between the exponential benefit of controlled multi-tasking and the small linear cost of control molecules makes it likely that evolution will have explored this option. Indeed, this may have been the only feasible way to lift the constraints on the complexity and sophistication of genetic programming.

Complex organisms require two levels of genetic programming for their autopoeitic development from a fertilised embryo. The genomes of these organisms must specify the functional components of the system, mainly proteins, which have been the primary focus of genetic and genomic research to date. Damage to these components (by mutation) is also very obvious (as in monogenic diseases), just as damaging the components of any structure is obvious. The genomes of these organisms must also specify the control architecture which deploys these components in sophisticated suites of differentiation and development. Damage to this architecture is much more subtle, because of the nature and complexity of this information (which primarily affects quantitative trait variation). Traditionally it has been assumed that this architecture is embedded in the cis-acting control sequences which regulate gene expression in conjunction with trans-acting proteins acting at a variety of levels. However, as noted above, the vast majority of the transcriptional output of the genomes of the higher organisms, up to 97-98% in humans, is noncoding RNA. This noncoding RNA is derived from the introns of both protein-encoding and non-protein-encoding (noncoding RNA) genes, and the exons of noncoding RNA genes, which appear to comprise at least half of all transcripts from the human genome. Putting together the extent of introns in protein coding genes with the estimate of the number of non-coding RNA genes suggests that at least 50% of the human genome is actively transcribed into non-coding RNAs. Thus, either that the human genome is replete with useless transcription or these RNAs are fulfilling some unexpected function(s).

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identifiers is provided in Table 1. A sequence listing is provided after the claims.

The present invention is predicated in part on the proposal that non-coding RNAs have evolved to form a second tier of gene expression in the eukaryotes, and that these molecules (or their processed derivatives) act as endogenous controls for genetic multitasking and regulating complex suites of gene expression. Since intronic RNAs are produced in parallel with protein encoding sequences, their most logical (general) function would be networking, i.e. a molecular memory of recent transcription events which allows activity at one locus to be communicated directly to others. If this is the case, then it can be predicted that these RNAs are further processed into multiple species, each one capable of transmitting information independently to different targets. This is similar to the types of networks that exist in other complex information systems such as the brain, where secondary outputs (termed efference signals) underlie sensory awareness, motor coordination, and cognition, and wherein the patterns of neural activation depend on the flux of “hidden units”, collectively referred to as the “hidden layer” (Mattick and Gagen. Molec. Biol. Evol. 18: 1611-1630, 2001). At face value, such efference RNAs (eRNAs) would enable an enormous increase in network connectivity and functionality over the situation where system activity is solely regulated through protein-based feedback loops which relay metabolic and environmental state information. They would also allow a much more sophisticated and genomically compact regulatory system than would be possible using proteins alone, especially for integrating the complex subroutines that operate during embryonic differentiation and development. Moreover, if a system utilizing an RNA communication network has evolved, it is also predicted that many genes have evolved solely to express RNA, as higher order regulators in the network. These noncoding RNAs would be expected to interact with, and to transmit signals to, a variety of cellular targets, including other RNAs, genes (DNA/chromatin), and proteins. It would also be predicted that a significant proportion of these interactions, perhaps the majority, would occur via sequence-specific interactions between the eRNAs (transmitters) and homologous target sequences in other RNAs or the genome (receivers), i.e. that the specificity of signalling is embedded in the primary sequence of the RNA transmitter and the RNA or DNA receiver as a kind of “bit string” or “zip code”. In both cases these transmitter and receiver sequences are encoded in the genome and potential interacting pairs within this regulatory network will be recognisable by sequence homology using rules that apply to duplex or higher order DNA-RNA or RNA-RNA interactions. In the case of RNA-protein interactions, the interacting partners will be identified by direct experimental procedures and/or ab initio from sequence analysis when the algorithms for this become available.

In accordance with the present invention, it is proposed that efference RNA signals integrate and regulate gene activity in eukaryotes at a variety of levels. It is also proposed that this RNA network was a fundamental advance in the genetic operating system of the eukaryotes, which lies at the heart of the programmed responses which direct cellular and differentiation and organismal development. At face value such a system has enormous advantages over a regulatory circuitry that relies simply on protein feedback loops, especially when attempting to integrate large sets and different levels of gene activity. If this is so, it further suggests that the evolution of a more advanced genetic operating system based on a highly parallel RNA-based communication network may have been the fundamental prerequisite for the emergence of complex organisms. It also implies that the basis of species diversity and quantitative trait variation in complex organisms is primarily embedded in the control architecture of the system, rather than structural variation in the protein components themselves (although this will also contribute). This in turn has considerable implications for understanding and modifying the genetic programming of the higher organisms and the genetic factors underpinning complex traits.

In accordance with the present invention therefore, it is proposed that RNA sequences derived from introns of protein-encoding genes and from introns and exons of non-protein-encoding transcripts have evolved to function as network control molecules in higher organisms, freeing such organisms from the constraints of a simple single-output protein-based genetic operating system. The recognition that such RNA sequences, referred to herein as efference or eRNAs, are genetic signalling modifiers permits the rational design of a range of signal modifiers including the identification of corresponding receiver DNA, RNA and protein molecules and permits rational modification of physiological, biochemical and genetic output to alter inter alia organismal differentiation and development to modify quantiative traits and to alter physiological parameters underlying disease and disease susceptibility. The recognition of the importance of eRNAs in defining the genetic architecture of a cell further enables cell and organismal programming or re-programming. This includes the identification and modification of eRNA transmitter sequences or their target sequences to alter the epigenetic status and accessiblity of genomic loci, gene transcription, alternative splicing, RNA turnover, mRNA translation and signal transduction systems. This is useful in directing the differentiation and development, for example of stem cells. It also enables the development of novel diagnostic and therapeutic protocols.

In addition, the present invention further enables the identification of embedded structural motifs which are involved in protein/RNA complex interaction.

The recognition that eRNAs and their receiver targets are involved in genetic network signalling permits the rational design of eRNAs and their analogs and to identify target sequences to thereby modulate genetic signalling pathways. The present invention enables, therefore, genetic engineering of cells at a highly sophisticated level. The present invention further provides a computer system for identifying eRNAs or DNA sequences encoding same as well as receiver DNA, RNA and proteins. Such a computer system includes software, hardware, computer codes, user interfaces and databases acquiring storing and retrieving genetic data and/or physiological or other biological data associated with eRNAs or DNAs encoding same.

Furthermore, the recognition of the role of eRNAs in determining the genetic architecture of a cell or group or family of cells, enables the design of protocols and genetic and chemical agents which can influence this architecture. Accordingly, agents can now be identified which can program a cell to differentiate, proliferate and/or re-new or re-program an already differentiated or partially differentiated cell to exhibit characteristics of another cell type.

The present invention provides, therefore, a method for modulating the genetic make up of a cell or the phenotype of a cell as well as agents useful for same. The present invention further enables high throughput screening protocols for agents which act via eRNAs or their receiver targets. Such agents include enogenous molecules such as RNA's or products identified by natural products screening or the screening of chemical libraries.

An example of eRNA is the shared intronic sequence of GRIA2, GRIA3 and GRIA4 genes shown in FIG. 6. The present invention extends to homologous eRNAs having at least 70% identity to the nucleotide sequence shown in FIG. 6 and to nucleotide sequences capable of hybridzing to the sequence shown in FIG. 6 or its complementary form under low stringency conditions.

The present invention is further useful in manipulating stem cells to differentiate along a particular pathway and, hence, be involved in tissue repair, regeneration and/or augmentation.

TAABLE 1
SUMMARY OF SEQUENCE IDENTIFIERS (SEQ ID Nos.)

| Seq ID No. | Description |
|---|---|
| 1 | Nucleotide sequence of intron from human Chr19 be- |
| | tween nucleotides 38234 and 167860 |
| 2-43 | Olgonucleotide human sequence enquiries |
| 44 | Nucleotide sequence of intron from human Chr12 be- |
| | tween 156966 and 180225 |
| 45-52 | Olgonucleotide human sequence enquiries |
| 53 | Nucleotide sequence of intron on human Chr12 between |
| | nucleotide 156966 and 180225 |
| 54-81 | Oligonucleotide sequence enquiries |
| 82-121 | Putative eRNA sequences for S. cerevisiae |

### BRIEF DESCRIPTION OF THE FIGURES

FIG. 1 is a schematic representation of sub-network, an uncontrolled regulated network and a controlled multi-tasked network. Panel (a) shows an uncontrolled sub-network wherein nodes take limited numbers of regulatory inputs rₖ and generate limited numbers of protein outputs gₖ. Here, g₁ regulates n₂ while being subject to feedback interactions from g₂ (dotted line). Panel (b) shows the same sub-network with each node expressing a multiplex output of protein product gₖ and many control molecules cₖ each capable of targeted interactions to multi-task the sub-network. A sample interactions (shown as dot-dash lines) include control c₁ determining the alternative splicing of the node n₃ output giving g₃ or g₃, the latter of which regulates node n₂ when expressed, while nodes n₁ and n₃ each feedback controls onto the other. It is evident that controls increase interconnectivity which increases network dynamical output complexity.

FIG. 2 is a diagrammatic representation showing (A) a simple network involved in particular cellular functions and (B) a complex network involved in cellular differentiation and development.

FIG. 3 is a diagrammatic representation of a system used to carry out the instructions encoded by the storage medium of FIGS. 4 and 5.

FIG. 4 is a diagrammatic representation of a cross-section of a magnetic storage medium.

FIG. 5 is a diagrammatic representation of a cross-section of an optically readable data storage system.

FIG. 6 is a diagrammatic representation of an eRNA network centred around the GRIA2, GRIA3 and GRIA4 genes. The eRNA comprises the nucleotide sequence which is a shared intronic sequence of the GRIA genes. The sequence is shown in the figure.

### DETAILED DESCRIPTION OF THE PREFERRED EMBODIMENTS

The present invention is predicated in part on the recognition that eukaryotic cells have evolved a complex network of genetic signals which facilitates integration of gene activity and multi-tasking of the cellular proteome. It is proposed, in accordance with the present invention, that integration and multi-tasking of this sophisticated and complex genetic network is mediated at least in part by trans-acting, non-protein coding RNA molecules corresponding to introns or other non-coding RNA sequences of protein-encoding nucleotide sequences or introns and/or exons from RNA sequences of non-protein-encoding nucleotide sequences. The identification of these RNA molecules, referred to herein as efference RNAs or eRNAs, permits the development of a further level of functional genomics and advanced genetic engineering. In particular, eRNAs and/or their target or associated molecules or homologs, analogs, functional equivalents or synthetic forms are now obtainable and have utility as therapeutic agents and trait-modifying agents in eukaryotic cells such as vertebrate and invertebrate animal cells and plant cells. The eRNAs and their targets influence, therefore, the genetic architecture of the cell and, hence, these molecules were as well as analogs and homologs thereof have trait-modification potential. Reference to a “target” includes a “receiver” and includes nucleotide sequences in genomic DNA or RNA, including introns, exons 5′ or 3′ untranslated regions of genes or their transcripts (UTRs), as well as 5′ or 3′ flanking regions of genes and intergenic regions, which act as receivers of the eRNAs. Such targets are referred to herein as “receiver DNAs” or “receiver RNAs”. The targets may also be proteins with which eRNAs interact (i.e. “receiver proteins”). The eRNAs are regarded as “transmitters”.

Accordingly, one aspect of the present invention contemplates a method for identifying an eRNA or a DNA sequence comprising an eRNA-encoding sequence in the nucleome of a eukaryotic cell, said method comprising identifying non-protein-encoding nucleotide sequences within an RNA transcript or a DNA sequence encoding same in said nucleome, determining the nucleotide sequence of said non-protein-encoding nucleotide sequence and subjecting said sequence to phenotyping to determine its effect on one or more biological events within a cell or an organism and/or determining the degree to which said sequence is conserved or is variant in the organism's genome or in the genome of other species or genera of eukaryotic cells wherein a non-protein-encoding nucleotide sequence having a biological effect in a cell or a nucleotide sequence conserved within the genome or between different cells' nucleomes is deemed to be an eRNA or DNA sequence comprising a nucleotide sequence encoding same.

In a related embodiment, there is provided a method for identifying a receiver DNA or RNA, said method comprising identifying an eRNA by the method comprising identifying non-protein-encoding nucleotide sequences within an RNA transcript or a DNA sequence encoding same in said nucleome, determining the nucleotide sequence of said non-protein-encoding nucleotide sequence and subjecting said sequence to phenotyping to determine its effect on one or more biological events within a cell or an organism and/or determining the degree to which said sequence is conserved or is variant in the organism's genome or in the genome of other species or genera of eukaryotic cells wherein a non-protein-encoding nucleotide sequence having a biological effect in a cell or a nucleotide sequence conserved within the genome or between different cells' nucleomes is deemed to be an eRNA or DNA sequence comprising a nucleotide sequence encoding same and then contacting said eRNA with nucleome material and screening for interaction between the eRNA and a DNA or RNA wherein the detection of such interaction is indicative of a receiver molecule.

In a further related embodiment, the present invention provides a method for identifying a receiver protein, said method comprising identifying an eRNA by the method comprising identifying non-protein-encoding nucleotide sequences within an RNA transcript or a DNA sequence encoding same in said nucleome, determining the nucleotide sequence of said non-protein-encoding nucleotide sequence and subjecting said sequence to phenotyping to determine its effect on one or more biological events within a cell or an organism and/or determining the degree to which said sequence is conserved or is variant in the organism's genome or in the genome of other species or genera of eukaryotic cells wherein a non-protein-encoding nucleotide sequence having a biological effect in a cell or a nucleotide sequence conserved within the genome or between different cells' nucleomes is deemed to be an eRNA or DNA sequence comprising a nucleotide sequence encoding same and then contacting said eRNA with proteome material and screening for interaction between the eRNA and a protein wherein the detection of such interaction is indicative of a receiver protein.

In an alternative embodiment, bioinformatics is used to identify conserved nucleotide sequences of putative eRNAs or receiver sequences. An example of a non-bioinformatic method to detect eRNAs and/or receiver molecules is by gel retardation assays.

An “eRNA” means an “efference RNA” and corresponds to an RNA derived from intronic sequences of protein-encoding genes or derived from intronic and/or exonic sequences of non-protein-encoding transcripts which are involved in endogenous control of a genetic network within eukaryotic cells, including modulation of signalling and genetic, events within and between eukaryotic cells to alter differentiation and development and to alter gene expression patterns that may be useful in advanced genetic engineering of plants, animals and other eukaryotes and in the treatment of imbalances that underlie common diseases including cancer. An eRNA is regarded herein as a transmitter. A non-protein-encoding transcript means an RNA sequence transcribed from a gene but which is not translated into a protein sequence. Reference to a “genetic network” includes the genetic signals required to inter alia induce expression of a suite of genes, induce physiological changes within, on or between cells or facilitate multi-tasking of a cell's proteome. The genetic network may also be regarded as the genetic architecture of the cell. Such networking may involve the facilitation of RNA-DNA, RNA-RNA and RNA-protein interactions and may readily be observed by parameters such as alterations to gene expression, RNA splicing, DNA methylation, remodelling of chromatin, other signal transduction systems and cellular physiology, including responses to environmental variables. eRNAs act inter alia via receiver DNA, RNA or protein sequences.

Reference to an “intron” includes any RNA sequence which is capable of being excised from a primary RNA transcript (e.g. a pre-messenger RNA transcript). An “exon” includes any RNA sequence which is re-assembled to form a contiguous RNA after the removal of introns by splicing, which may form a messenger RNA (mRNA) containing protein-coding sequence, or a non-protein-coding RNA without protein-coding capacity. “Non-protein-encoding RNA sequences” also includes introns as well as RNA sequences 5′ of the authentic translation initiation site or 3′ of the translation termination codon. The latter two sites are generally referred to 5′ untranslated regions (UTR) or 3′ UTR of mRNA. The term “untranslated region” or “UTR” is a term of the art referring to the particular location of a genetic sequence relative to the translation initiation site. However, the use of these terms is not to exclude the possibility that some partial translation may occur in this region. For convenience, reference to a “protein” includes reference to a peptide or polypeptide. In a particularly preferred embodiment, the 3′ and 5′ UTRs or parts thereof act as receiver molecules for eRNAs.

An “RNA transcript” represents the sequence of ribonucleotides transcribed from a deoxyribonucleotide sequence of a gene. Thus, an RNA transcript includes and encompasses a primary gene transcript or pre-messenger RNA (pre-mRNA), which may contain one or more introns, as well as a messenger RNA (mRNA) in which any introns of the pre-mRNA have been excised and the exons spliced together. It is proposed, in accordance with the present invention, that some of the excised RNA introns in protein-coding transcripts or introns and exons in non-protein-coding transcripts act as eRNA molecules and modulate genetic signalling within a cell.

The “proteome” is regarded as the total protein within and on a cell. The “nucleome” is the total nucleic acid complement and includes the genome and all RNA molecules such as mRNA, heterogenous nuclear RNA (hnRNA), small nuclear RNA (snRNA), small nucleolar RNA (snoRNA), small cytoplasmic RNA (scRNA), ribosomal RNA (rRNA), translational control RNA (tcRNA), transfer RNA (tRNA), eRNA, messenger-RNA-interfering complementary RNA (micRNA) or interference RNA (iRNA) and mitochondrial RNA (mtRNA).

It is particularly useful to identify eRNAs on the basis of conserved ribonucleotide sequences in intronic RNA sequences of protein-encoding nucleotide sequences or intronic and/or exonic sequences of non-protein-encoding nucleotide sequences or their corresponding deoxyribonucleotide sequences. Reference to “conserved” includes any polyribonucleotide or polydeoxyribonucleotide sequence sharing at least about 80% nucleotide complementarity to another sequence in the nucleome. Conserved sequences in the genome including 3′ and 5′ regions of genes is suggestive of a putative receiver molecule.

The term “similarity” as used herein includes partial or exact sequence identity or complementarity between compared sequences at the nucleotide level. In a preferred embodiment, nucleotide and sequence comparisons are made at the level of exact complimentarity or identity rather than partial identity or complementarity.

Terms used to describe sequence relationships between two or more polynucleotides include “reference sequence”, “comparison window”, “sequence similarity”, “sequence identity”, “sequence complementarity”, “percentage of sequence similarity”, “percentage of sequence identity”, “percentage of sequence complementarity”, “substantial similarity”, “substantial complementarity” and “substantial identity”. A “reference sequence” is at least 12 but frequently 15 to 18 and often at least 25 or above, such as 30 monomer units, inclusive of nucleotides, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity or complementarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. Nucl. Acids Res. 25: 3389 1997. A detailed discussion of sequence analysis can be found in Unit 19.3 of Ausubel et al. (1998).

The terms “sequence similarity”, “sequence identity” and “sequence complementarity” as used herein refers to the extent that sequences are identical or functionally or structurally similar or complementary on a nucleotide-by-nucleotide basis over a window of comparison using standard rules for DNA-DNA, RNA-RNA and RNA-DNA base pairing. Thus, a “percentage of sequence identity”, for example, is calculated by comparing two optimally aligned sequences over the window of comparison, determining the number of positions at which the identical nucleic acid base (e.g. A, T, C, G, I, U) occurs in both sequences to yield the number of matched positions, dividing the number of matched positions by the total number of positions in the window of comparison (i.e., the window size), and multiplying the result by 100 to yield the percentage of sequence identity or complementarity. For the purposes of the present invention, “sequence identity” between DNA sequences will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to DNA sequence similarity. Sequence complementarity in duplex and higher order RNA-RNA, RNA-DNA and RNA-protein interactions will be assessed by rules as described in Hermann. et al., Chem Biol, 6: R335-43. 1999; Masquida et al. Rna, 6: 9-15. 2000; Praseuth et al., Biochim Biophys Acta, 1489: 181-206 1999; Varani et al., EMBO Rep, 1: 18-23 2000.

Conveniently, an intronic or other protein-non-encoding sequence at the RNA or DNA level to a database of DNA or RNA sequences in the genome or nucleome and the identification of at least 80% similar sequences (e.g. determined by BLAST analysis) after optimal alignment is determined. The presence of one or more other homologous or complementary sequences in the database or between databases for different species, genera or families of invertebrate or non-invertebrate animals or plants is indicative of a candidate sequence involved in genetic network signal modulation.

Sequence similarity and complementarity provides one of a number of features or identifiers useful for analyzing the likelihood of a target RNA sequence being an eRNA. Other identifiers include the participation of the gene from which the potential eRNA is derived in a pathway or its involvement in multiple pathways such as part of the physiological or genetic networks contained within a cell. Furthermore, putative eRNA sequences may also share common secondary or tertiary structures. This may occur, for example, when the eRNA interacts with certain RNAses or ribosomes or nucleic acid binding proteins. Partly as a result of these features, apart from sequence determination, putative eRNA sequences may be detected by conventional genetic techniques such as deletional analysis, transgenesis, genetic silencing procedures (e.g. co-suppression, antisense techniques, RNAi induction) and the physiological effects of such procedures observed. Such physiological effects are referred to herein as a nucleotide sequence having a “biological effect”. Furthermore, the effect of eRNA may be demonstrated by ectopic expression studies. For example, intronic sequences from protein-coding sequences may be expressed on non-protein-coding sequences to determine the function of the eRNA in the absence of exon sequences or cis-acting elements in the transcript from which the eRNA is obtained. Transgenic animals and cells obtained therefrom in which genomic sequences have been replaced by cDNA sequences which do not contain the introns of the genetic sequences can also be employed.

The main advantage of RNA as a regulatory molecule is its compact size and sequence specificity. The likelihood is that most RNA signals will be transmitted through primary sequence-specific interactions with other RNAs and with DNA, forming complexes that are recognized by proteins containing particular types of domains. This provides an opportunity to identify both the potential transmitters and receivers (targets) in such networks, as well as the types of interacting proteins. Importantly, most of these interactions would be expected to involve RNA-RNA and RNA-DNA interactions (potentially including triplexes and other higher-order structures) that do not obey canonical Watson-Crick base-pairing rules. Thus, the present invention extends to algorithms which allow genomic sequence to be searched for these different types of interactions. Complete search algorithms, such as those based on suffix arrays and suffix trees are particularly useful to analyse this properly.

The ability of RNA to form strong interactions with other RNAs suggests that RNA-RNA and (to a lesser extent) RNA-DNA base pairing is stronger than DNA-DNA base pairing, and can allow for stable mismatches and the formation of particular secondary structures such as bulges, stems and loops, which, rather than being seen as mismatch errors (as in DNA repair), may also in fact contain embedded structural motifs that can be recognized by particular proteins. For example, perfect versus imperfect matching of microRNAs to their targets determines whether the mRNA target is actively degraded by the RNAi pathway or is translationally repressed.

Accordingly, it is proposed that the prediction can be made that different types of RNA signals and the different structures of the resulting complexes are recognized and acted on by particular classes of nucleic-acid-binding proteins. An understanding these secondary structural and mismatch rules enables the bioinformatic approaches to dissecting these networks at the genomic level. It also allows better prediction of the regulatory consequences of different types of RNA signals, by the development of specific algorithms to identify particular subsets that obey different sets of rules for the combination of sequence specificity and the type of secondary structure that is created by the interaction, bearing in mind that parts of the network will be silent in any given cell or lineage because an RNA transmitter or target is not expressed, or a DNA target has been made inaccessible by chromatin modification.

The present invention is predicated in part on the proposal that in order for a molecular genetic network to be capable of complex programming and multi-tasking, each of the gene sub-networks within a cell must produce numerous control molecules in parallel with their primary gene products, which dynamically communicate with other sub-networks (via transcriptional, splicing and translational controls, among others). Such a system would be expected to display an exponential increase in its ability to manage and integrate larger genetic datasets, and in its functionality and phenotypic range. In addition, because modulation of system dynamics can be readily achieved by mutation of control molecules, such a system should be able to explore new expression space at fast evolutionary rates over short evolutionary timescales.

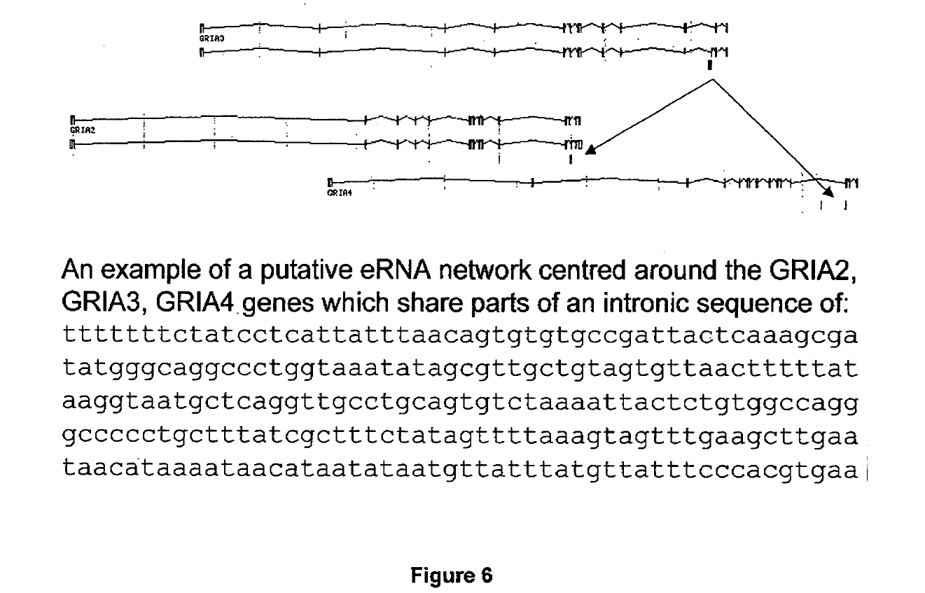

An example of eRNA is the shared intronic sequence of GRIA2, GRIA3 and GRIA4 genes shown in FIG. 6. The present invention extends to homologous eRNAs having at least 70% identity to the nucleotide sequence shown in FIG. 6 and to nucleotide sequences capable of hybridzing to the sequence shown in FIG. 6 or its complementary form under low stringency conditions.

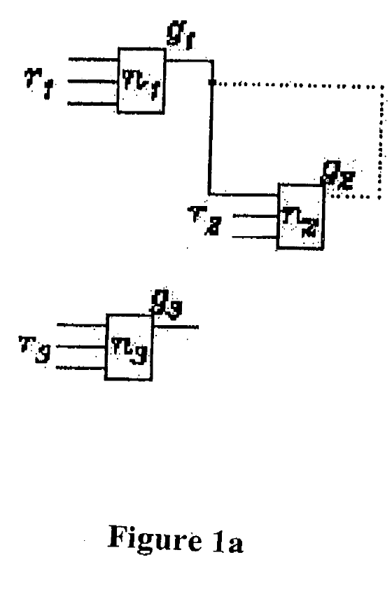

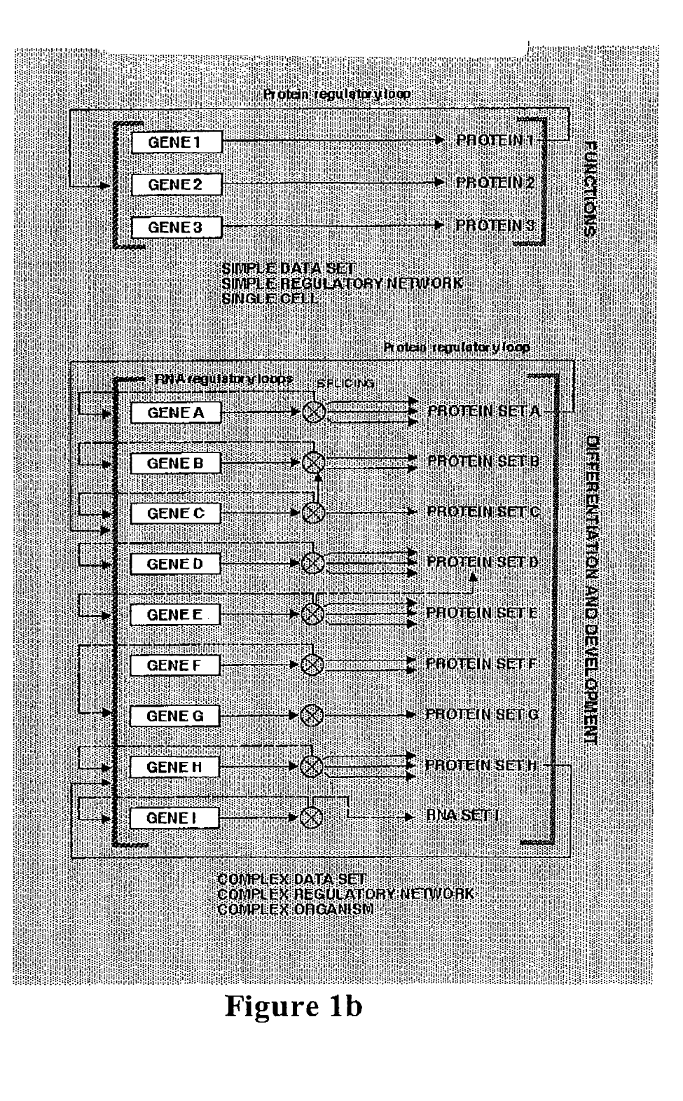

A controlled multi-tasked molecular network is schematically shown in FIG. 1, in contrast to an uncontrolled regulated network. This network architecture can be equally applied to computer networks, neural networks and cellular networks. An example of simple and complex genetic networks is shown in FIG. 2.

The nodes of a controlled multi-tasked network must be capable of generating and integrating multiple inputs and outputs. Such networks are generally stable and scale-free, with some nodes having high connectivity and others low connectivity, similar to most communication and social networks, including the Internet (Albert et al., Nature 406: 378-382, 2000). Multiply connected networks are widely employed in other complex information processing systems, including in neurobiology where secondary networking signals, termed “efference” signals, underlie sensory awareness and motor coordination (Bridgeman, Ann. Biomed. Eng. 23: 409-422 1995; Andersen et al., Annu. Rev. Neurosci 20: 303-330 1997). The concept of multiple inputs and outputs is also a well established feature of neural networks in cognition, language and memory (Plunkett et al., J. Child Psychol. Psychiatry 38: 53-80 1997; Elman, A Companion to Cognitive Science, Basil Blackwood Bechtel and Graham, Eds 1998). These networks involve densely connected webs of processing units that propagate and transform complex patterns of activity, and are capable of self-organization. They operate by a form of parallel distributed processing, whereby information is distributed across the system such that patterns of activation across sets of “hidden units” (i.e. controls), which define the state of the network, then determine the pattern of activation across output nodes (McClelland and Rumelhart, J. Exp. Psychol. Gen 114: 159-197 1985; McClelland and Plaut, Curr. Opin. Neurohol 3: 209-216 1993; Plunkett et al., J. Child Psychol. Psychiatry 38: 53-80 1997).

The assessment of the presence of similar nucleotide sequences in a genome or nucleome database is suitably facilitated with the assistance of a computer programmed with software, which inter alia adds or weighs index values (I_{V}) for each feature associated with the candidate sequences to provide a predictive value (P_{V}) corresponding to the likelihood of the candidate sequences being involved in modulating genetic network signalling. The features are selected from:—

(a) the transmitter sequence is derived from an intron in a protein-coding RNA transcript or an intron or an exon in a non-protein-coding RNA transcript or their DNA equivalents;

(b) the target receiver sequence lies in an intron or an exon in an RNA transcript or its DNA equivalent;

(c) the target receiver sequence lies in an intergenic genomic DNA sequence, such as a promoter or enhancer region;

(d) the target receiver is a DNA or RNA sequence capable of interaction with an eRNA;

(e) the target receiver sequence lies in a 5′ untranslated region of an RNA transcript or its DNA equivalent;

(f) the target receiver sequence lies in a 3′ untranslated region of an RNA transcript or its DNA equivalent;

(g) the target receiver is a protein capable of sequence-specific recognition of an eRNA and/or its target recognition sequences;

(h) the sequence is a DNA or RNA which recognizes and/or interacts with an eRNA;

(i) the sequence comprises at least 12 nucleotides;

(j) the sequence has at least 80% nucleotide identity or complementarity to at least one sequence of the same genome or nucleome;

(k) the sequence has at least 80% nucleotide identity or complementarity to at least one sequence in a genome or nucleome of a different species, genus or family of animal or plant cells;

(l) The sequence associates by its position to a feature from available databases, for example, Genbank, the Gene Ontology databse or SWISSPORT; and

(m) The sequence associates by its position to a protein (ie. falls within the transcript) and that protein's expression profile, as determined by microarray analysis, is modulated in a specific way during a phenomona of interest, for example, highly up or down regulated in the initial phase of meiosis.

In a preferred embodiment of the features (j) and (k), the sequence preferably has at least 90% and more preferably at least 95% nucleotide identity or complementarity to said at least one sequence (e.g. as determined by BLAST analysis) such as at least about 96%, 97%, 98%, 99% or 100%.

With respect to feature (i), the preferred number of nucleotides is from about 12 to about 100, more preferably from about 12 to about 50 and even more preferably from about 12 to about 30 such as about 22.

Preferably, the features are further selected from:—

(1) expression of the sequences mentioned in (e) is associated with the modulation of the same phenotype.

In accordance with the present invention, index values for such features are stored in a machine-readable storage medium which is capable of being processed by the processing means of the computer to provide a predictive value for a candidate sequence being involved in genetic regulation.

Thus, in another aspect, the invention contemplates a computer program product for assessing the likelihood of a candidate nucleotide sequence or group of nucleotide sequences being an eRNA or a receiver for an eRNA involved in network genetic signalling, said product comprising:—

(1) code that receives as input index values for one or more of features wherein said features are selected from:

(a) the transmitter sequence is derived from an intron in a protein-coding RNA transcript or an intron or an exon in a non-protein-coding RNA transcript or their DNA equivalent;

(b) the target receiver sequence lies in an intron or an exon in an RNA transcript or its DNA equivalent;

(c) the target receiver sequence lies in an intergenic genomic DNA sequence, such as a promoter or enhancer region;

(d) the target receiver is a DNA or RNA sequence capable of interaction with an eRNA;

(e) the target receiver sequence lies in a 5′ untranslated region of an RNA transcript or its DNA equivalent;

(f) the target receiver sequence lies in a 3′ untranslated region of an RNA transcript or its DNA equivalent;

(g) the target receiver is a protein capable of sequence-specific recognition of an eRNA and/or its target recognition sequences;

(h) the sequence is a DNA or RNA which recognizes and/or interacts with an eRNA;

(i) the sequence comprises at least 12 nucleotides;

(j) the sequence has at least 80% nucleotide identity or complementarity to at least one sequence of the same genome or nucleome;

(k) the sequence has at least 80% nucleotide identity or complementarity to at least one sequence in a genome or nucleome of a different species, genus or family of animal or plant cells;

(l) the sequence associates by its position to a feature from available databases, for example, Genbank, the Gene Ontology database, SWISSPORT

(m) The sequence associates by its position to a protein (ie. falls within the transcript) and that protein's expression profile, as determined by microarray analysis, is modulated in a specific way during a phenomona of interest, for example highly up or down regulated in the initial phase of meiosis.

(2) code that adds said index values to provide a sum corresponding to a predictive value for said candidate sequences; and

(3) a computer readable medium that stores the codes.

In a related embodiment, the present invention is directed to a computer program product for assessing the likelihood of a candidate nucleotide sequence or group of nucleotide sequences being a receiver molecule involved in network signalling via an eRNA, said product comprising:—

(1) code that receives as input index values for one or more of features wherein said features are selected from:—

(a) the target receiver sequence lies in an intergenic genomic DNA sequence, such as a promoter or enhancer region;

(b) the target receiver is a DNA or RNA sequence capable of interaction with an eRNA;

(c) the target receiver sequence lies in a 5′ untranslated region of an RNA transcript or its DNA equivalent;

(d) the target receiver sequence lies in a 3′ untranslated region of an RNA transcript or its DNA equivalent;

(e) the target receiver is a protein capable of sequence-specific recognition of an eRNA and/or its target recognition sequences;

(f) the sequence is a DNA or RNA which recognizes and/or interacts with an eRNA;

(g) the sequence comprises at least 12 nucleotides;

(h) the sequence has at least 80% nucleotide identity or complementarity to at least one sequence of the same genome or nucleome;

(i) the sequence has at least 80% nucleotide identity or complementarity to at least one sequence in a genome or nucleome of a different species, genus or family of animal or plant cells;

(j) The sequence associates by its position to a feature from available databases, for example, Genbank, the Gene Ontology database, SWISSPORT;

(k) The sequence associates by its position to a protein (ie. falls within the transcript) and that proteins expression profile, as determined by microarray analysis, is modulated in a specific way during a phenomona of interest, for example highly up or down regulated in the initial phase of meiosis.

(2) code that adds said index values to provide a sum corresponding to a predictive value for said candidate sequences; and

(3) a computer readable medium that stores the codes.

In a preferred embodiment, the computer program product comprises codes which assign an index value for each feature of a candidate sequence.

In a related aspect, the invention extends to a computer system for assessing the likelihood of a candidate sequence or group of candidate sequences being an eRNA involved in network genetic signalling wherein said computer system comprises:—

(1) a machine-readable data storage medium comprising a data storage material encoded with machine-readable data, wherein said machine-readable data comprise index values for one or more features, wherein said features are selected from:—

(a) the transmitter eRNA sequence is derived from an intron in a protein-coding RNA transcript or an intron or an exon in a non-protein-coding RNA transcript, or their DNA equivalent;

(b) the sequence comprises at least 12 nucleotides;

(c) the sequence has at least 80% nucleotide identity or complementarity to at least one sequence of the same genome or nucleome;

(d) the sequence has at least 80% nucleotide identity or complementarity to at least one sequence in a genome or nucleome of a different species, genus or family of animal or plant cells;

(e) the sequence comprises a secondary or tertiary structure having an activity; and

(f) the sequence exhibits catalytic activity;

(2) a working memory for storing instructions for processing said machine-readable data;

(3) a central-processing unit coupled to said working memory and to said machine-readable data storage medium, for processing said machine readable data to provide a sum of said index values corresponding to a predictive value for said candidate sequences; and

(4) an output hardware coupled to said central processing unit for receiving said predictive value.

Even yet another aspect of the invention extends to a computer system for assessing the likelihood of a candidate sequence or group of candidate sequences being a receiver RNA, DNA or protein involved in network genetic signalling wherein said computer system comprises:—

(1) a machine-readable data storage medium comprising a data storage material encoded with machine-readable data, wherein said machine-readable data comprise index values for one or more features, wherein said features are selected from:—

(a) the sequence is located in an intron or an exon in an RNA transcript or its DNA equivalent;

(b) the target receiver sequence lies in an intergenic genomic DNA sequence, such as a promoter or enhancer region;

(c) the sequence is located in a 5′ untranslated region of an RNA transcript or its DNA equivalent;

(d) the sequence is located in a 3′ untranslated region of an RNA transcript or its DNA equivalent;

(e) the sequence is a protein capable of sequence-specific recognition of an eRNA and/or its target recognition sequence;

(f) the sequence is an RNA or DNA which recognizes and/or interacts with an eRNA;

(g) the sequence comprises at least 12 nucleotides;

(h) the sequence has at least 80% nucleotide identity or complementarity to at least one sequence of the same genome or nucleome;

(i) the sequence has at least 80% nucleotide identity or complementarity to at least one sequence in a genome or nucleome of a different species, genus or family of animal or plant cells;

(j) the sequence comprises a secondary or tertiary structure having an activity; and

(k) the sequence exhibits catalytic activity;

(2) a working memory for storing instructions for processing said machine-readable data;

(3) a central-processing unit coupled to said working memory and to said machine-readable data storage medium, for processing said machine readable data to provide a sum of said index values corresponding to a predictive value for said candidate sequences; and

(4) an output hardware coupled to said central processing unit for receiving said predictive value.

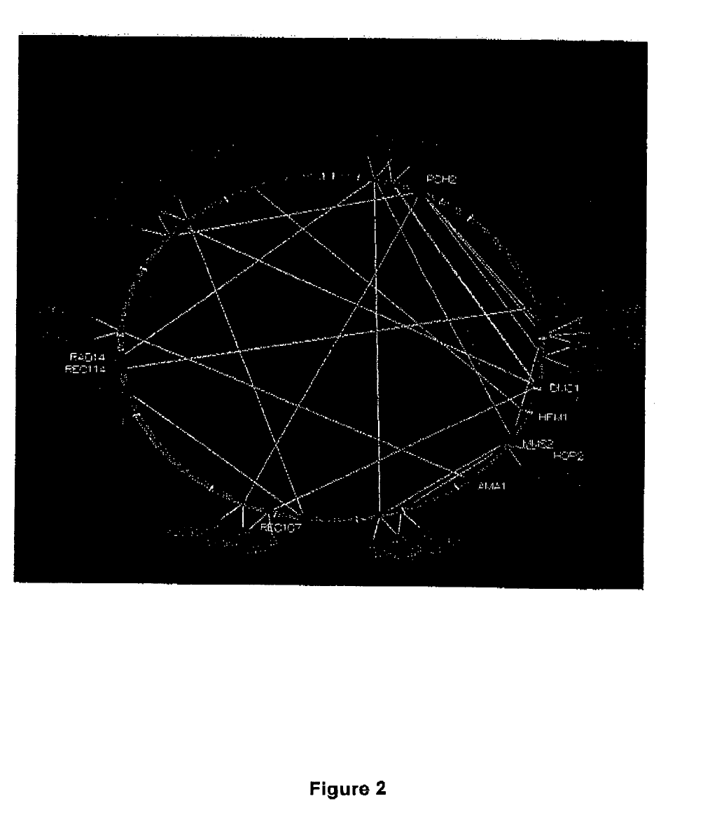

A version of these embodiments is presented in FIG. 3, which shows a system 10 including a computer 11 comprising a central processing unit (“CPU”) 20, a working memory 22 which may be, e.g. RAM (random-access memory) or “core” memory, mass storage memory 24 (such as one or more disk drives or CD-ROM drives), one or more cathode-ray tube (“CRT”) display terminals 26, one or more keyboards 28, one or more input lines 30, and one or more output lines 40, all of which are interconnected by a conventional bidirectional system bus 50.

Input hardware 36, coupled to computer 11 by input lines 30, may be implemented in a variety of ways. For example, machine-readable data of this invention may be inputted via the use of a modem or modems 32 connected by a telephone line or dedicated data line 34. Alternatively or additionally, the input hardware 36 may comprise CD. Alternatively, ROM drives or disk drives 24 in conjunction with display terminal 26, keyboard 28 may also be used as an input device.

Output hardware 46, coupled to computer 11 by output lines 40, may similarly be implemented by conventional devices. By way of example, output hardware 46 may include CRT display terminal 26 for displaying a synthetic polynucleotide sequence or a synthetic polypeptide sequence as described herein. Output hardware might also include a printer 42, so that hard copy output may be produced, or a disk drive 24, to store system output for later use.

In operation, CPU 20 coordinates the use of the various input and output devices 36,46 coordinates data accesses from mass storage 24 and accesses to and from working memory 22, and determines the sequence of data processing steps. A number of programs may be used to process the machine readable data of this invention. Exemplary programs may use for example the following steps:—

(1) inputting index values for at least one feature associated with a candidate sequence, wherein said features are selected from:—

(a) the sequence is an intron or exon in an RNA transcript or its DNA equivalent;

(b) the sequence is a 5′ untranslated region of an RNA transcript or its DNA equivalent;

(c) the sequence is a 3′ untranslated region of an RNA transcript or its DNA equivalent;

(d) the sequence is a DNA, RNA or protein which is capable of interaction with an eRNA;

(e) the sequence comprises at least 12 nucleotides;

(f) the sequence has at least 80% nucleotide identity or complementarity to at least one sequence of the same genome or nucleome;

(g) the sequence has at least 80% nucleotide identity or complementarity to at least one sequence in a genome or nucleome of a different species, genus or family of animal or plant cells;

(h) the sequence comprises a secondary or tertiary structure having an activity; and

(i) the sequence exhibits catalytic activity;

(2) adding the index values for said features to provide a predictive value for said sequence; and (3) outputting said predictive value.

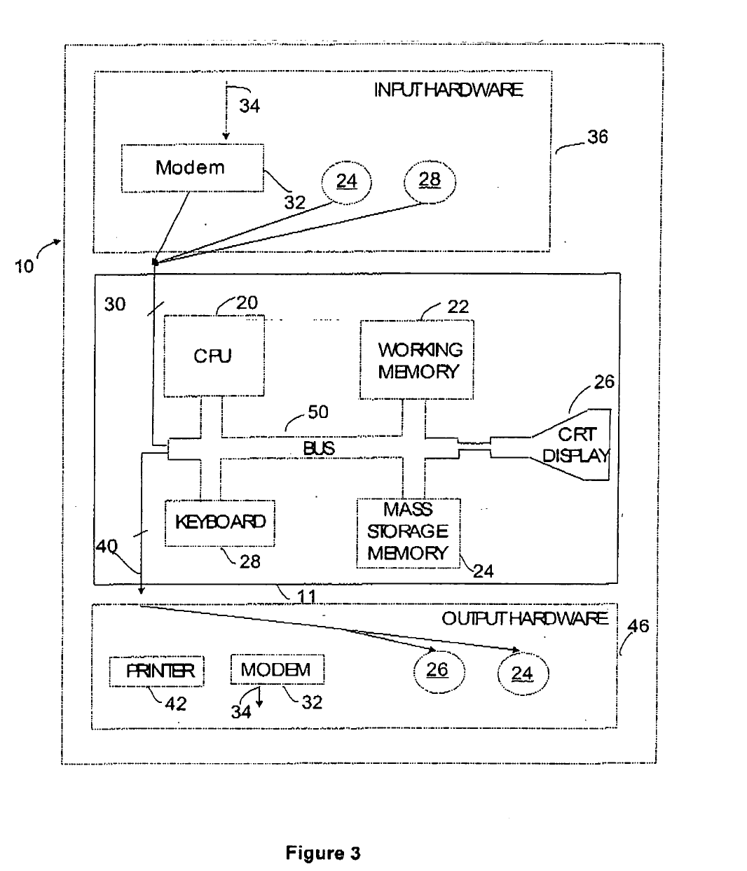

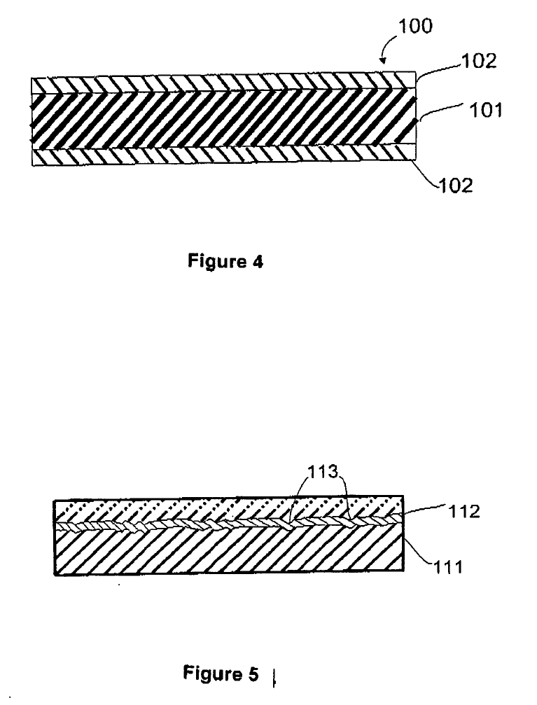

FIG. 4 shows a cross section of a magnetic data storage medium 100 which can be encoded with machine readable data, or set of instructions, for designing a synthetic molecule of the invention, which can be carried out by a system such as system 10 of FIG. 5. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24. The magnetic domains of coating 102 of medium 100 are polarized or oriented so as to encode in manner which may be conventional, machine readable data such as that described herein, for execution by a system such as system 10 of FIG. 3.

FIG. 4 shows a cross section of an optically readable data storage medium 110 which also can be encoded with such a machine-readable data, or set of instructions, for screening a candidate molecule of the present invention, which can be carried out by a system such as system 10 of FIG. 3. Medium 110 can be a conventional compact disk read only memory (CD-ROM) or a rewritable medium such as a magneto-optical disk, which is optically readable and magneto-optically writable. Medium 100 preferably has a suitable substrate 111, which may be conventional, and a suitable coating 112, which may be conventional, usually of one side of substrate 111.

In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarisation of laser light reflected from coating 112. The arrangement of the domains encodes the data as described above.

In essence, the subject computer software analyzes genomic or nucleomic databases for the presence of particular sequences which have one or more features as defined above. Each of these features carries a certain weight as to the importance in establishing that a target sequence is an eRNA or is a DNA sequence encoding an eRNA. Multiple features may be created by combining the features with certain biological effects as discussed above. For example, a conserved intron between species may combine with certain biological phenomena associated with a conserved deletion of this sequence. The resulting features, sub-features and multiple features and combinations thereof combine to produce a “fingerprint” or “descriptor” of not only an individual eRNA but also families of eRNAs and this may also provide a fingerprint of the gene expression status of a cell or animal or plant comprising cells at any given time.

The present system retrieves features and forms composite features from them. More than one feature can be combined in a variety of different ways to form these composite features. In particular, the composite feature can be any function or combination of a simple feature and other composite features. The function can be algebraic, logical, sinusoidal, logarithmic, linear, hyperbolic, statistical and the like. Alternatively, more than one feature can be obtained in a functional manner (e.g. arithmetic, algebraic). By way of example, a composite feature may equal the sum of two or more features or a composite feature may correspond to a sub-fraction of overlap of one or more features from another feature. Alternatively, a composite feature may equal a constant times one or more features. Of course, there are many other ways composite features can be defined.

The genome/nucleome databases may be from any eukaryotic cell such as from a vertebrate or invertebrate, including mammalian, avian, reptilian and amphibian animals, as well as from plants. The term “plants” includes monocotyledonous and dicotyledonous plants. It is particularly useful to employ the analysis function aspect of the present invention to human genome databases.

Computer programs may also be designed to screen nucleic acid molecule similarity at the secondary or tertiary levels. Furthermore, epidemiological studies together with polymorphism mapping may identify conserved polymorphisms in otherwise non-homologous nucleotide sequences. This would suggest an eRNA which is active at the secondary or tertiary levels.

Although not intending to limit the present invention to any one theory or mode of action, it is proposed that the eRNA molecules are “eRNA senders” or “eRNA transmitters” in the sense that they function as trans-acting networking molecules. eRNA senders have target molecules in the form of DNA, RNA and protein receivers. The receiver molecules may be located anywhere in the proteome, genome or nucleome. The identification of an eRNA permits the identification of these receiver molecules. Furthermore, again not intending to limit the present invention to any one theory or mode of action, it is proposed that there may be a connection between interference RNA (RNAi) and eRNA. RNAi is induced by, for example, double standard RNA generally corresponding to at least part of a coding strand of a gene. It is proposed, herein, that eRNAs may also induce RNAi and in fact be the true inducer of RNAi.

Consequently, another aspect of the present invention contemplates a method of inducing post transcription gene silencing (PTGS) of a gene carrying a nucleotide receiver sequence, said method comprising expressing an eRNA having said receiver nucleotide sequence which induces an RNAi capable of targeting said receiver sequence in an mRNA transcript of said gene. The ability to induce specific RNAi mediated PTGS or transcriptional gene silencing (TGS) using eRNAs or their homologs or analogs will greatly enhance the ability to modify traits in plant and animal cells.

RNAi, both in therapeutic and experimental usage, is complicated by an effect known as RNAi transitivity. When a gene is silenced by a RNAi signal, if the transcript of the gene has within it a sequence exactly homologous to the transcript of another gene it is possible for the second gene to be silenced as well, an effect which could lead to invalid experimental results or side-effects in therapy.

Thus, another aspect of the present invention is the utilization of eRNA networks to predict the scope and effect of transitive RNAi, by analysing the sequence of the targeted gene and comparing it to known effectors in the gene regulatory network.

Another aspect of the present invention provides an eRNA molecule identified by the method comprising identifying non-protein-encoding nucleotide sequences within an RNA transcript or a DNA sequence encoding same in said nucleome, determining the nucleotide sequence of said non-protein-encoding nucleotide sequence and subjecting said sequence to phenotyping to determine its effect on one or more biological events within a cell and/or determining the degree to which said sequence is conserved in the cell's genome or in the genome of other species or genera of eukaryotic cells wherein a non-protein-encoding nucleotide sequence having a biological effect in a cell or a nucleotide sequence conserved within the genome or between different cells' nucleomes is deemed to be an eRNA or DNA sequence comprising a nucleotide sequence encoding same.

Yet another aspect of the present invention is directed to a receiver DNA or RNA identified by the method comprising identifying non-protein-encoding nucleotide sequences within an RNA transcript or a DNA sequence encoding same in said nucleome, determining the nucleotide sequence of said non-protein-encoding nucleotide sequence and subjecting said sequence to phenotyping to determine its effect on one or more biological events within a cell and/or determining the degree to which said sequence is conserved in the cell's genome or in the genome of other species or genera of eukaryotic cells wherein a non-protein-encoding nucleotide sequence having a biological effect in a cell or a nucleotide sequence conserved within the genome or between different cells' nucleomes is deemed to be an eRNA or DNA sequence comprising a nucleotide sequence encoding same and then contacting said eRNA with nucleome material and screening for interaction between the eRNA and a DNA, RNA or protein wherein the detection of such interaction is indicative of a receiver molecule.

Still another aspect of the present invention provides a receiver protein identified by the method comprising identifying non-protein-encoding nucleotide sequences within an RNA transcript or a DNA sequence encoding same in said nucleome, determining the nucleotide sequence of said non-protein-encoding nucleotide sequence and subjecting said sequence to phenotyping to determine its effect on one or more biological events within a cell and/or determining the degree to which said sequence is conserved in the cell's genome or in the genome of other species or genera of eukaryotic cells wherein a non-protein-encoding nucleotide sequence having a biological effect in a cell or a nucleotide sequence conserved within the genome or between different cells' nucleomes is deemed to be an eRNA or DNA sequence comprising a nucleotide sequence encoding same and then contacting said eRNA with proteome material and screening for interaction between the eRNA and a protein wherein the detection of such interaction is indicative of a receiver protein.

Determination of methylation profiles within a cell and more particularly changing profiles in differentiating, aging or mutating cells is a convenient way of identifying epigenetic signatures in the genome and therefore identifying putative genetic targets for the presence of putative eRNAs or their corresponding receiver sequences.

One convenient method is described in an International Application filed 14 Sep. 2002 in the name of The University of Queensland and involves an amplification-based assay procedure to determine the methylation profile of nucleotides in the genome of a cell or group of cells. More particularly, the nucleotides are in the form of CpG or CpNpG sites. The ability to determine genomic and transgene methylomes in a cell or group of cells is an important tool in functional genomics and in developing the next generation of gene-expression modulating agents. Combining methylation profile with mapping enables a determination of the epigenetic consequences of internal and external stimuli. For example, methylation profiles may correlate with disease conditions or a propensity for a disease condition to develop or monitoring the aging process or the development process of cells. Furthermore, the methylation profile can be used to determine genes which either are expressed or are not expressed in certain disease states or with certain phenotypic traits. The identification of a condition or predisposition for development of a condition leads to the selection of targets for the identification of eRNAs or receiver sequences for eRNAs.

The amplification-based technology is referred to as amplified methylation polymorphisms (AMP). The AMP technology determines the methylation profile of many thousands of CpG or CpNpG sites around the genome and provides a genetic profile of the methylation status of these sites. This genetic signature is the methylome fingerprint of a cell's or group of cells' genome.

The AMP technology involves amplification of DNA markers in the form of small inverted repeats comprising the CpG or CpNpG sites but where amplification depends on the methylation status of the cytosines within the amplicon or nearby.

The protocol uses, in one form, a single arbitrary decamer oligonucleotide primer containing the recognition sequences of a methylation-sensitive restriction enzyme. These short oligonucleotide primers containing such recognition sequences are referred to herein as AMP primers. The recognition sequences for the methylation-sensitive restriction enzyme are located in the middle of the primer followed by up to four selective nucleotides, extending to the 3′ end. AMP profiles are generated from both undigested genomic DNA and genomic DNA digested with the methylation sensitive enzyme. Comparison of the profiles from digested and undigested genomic DNA reveals three classes of AMP markers: digestion resistant (Class I) indicative of methylation, digestion sensitive (Class II) indicative of non-methylation, and digestion dependent (Class III). The nature of the last class of AMP markers is proposed to represent physically-linked cis-acting inhibitory sequences which suppress amplification of Class III markers from undigested template. Digestion with the enzyme removes the inhibitor from the amplicon, thereby allowing amplification. The digestion-dependent (Class III) markers are proposed to encompass a methylated restriction site or sites in the amplicon sequence flanked by a non-methylated restriction site and then the putative inhibitory sequence. Digestion-dependent markers represent, therefore, junctions between methylated and non-methylated DNA in the genome. Cloning, sequencing and mapping AMP markers shows that they often correspond to CpG islands, features known to be landmarks for genes in genomes. These are then proposed to be sites of eRNA or eRNA receiver systems.

Methylation enzymes contemplated herein include AatII, AciI, AclI, AgeI, AscI, AvaI, BamHI, BsaA1, BsaH1, BsiE, BsiW, BsrF, BssHII, BstBI, BstUI, Cla1, EagI, HaeII, HgaI, HhaI, HinPI, HpaII, MloI, MspI, NaeI, NarI, NotI, NruI and PmlI. HpaII is particularly preferred in accordance with the present invention.

Accordingly, another aspect of the present invention provides a method for identifying a gene having encoding a putative eRNA or comprising a receiver sequence for an eRNA said method comprising determining the methylation profile of one or more CpG or CpNpG nucleotides at one or more sites within the genome of a eukaryotic cell or group of cells by obtaining a sample of genomic DNA from the cell or group of cells, digesting a sub-sample of the sample of genomic DNA with HpaII which has a recognition nucleotide sequence corresponding to or within the sites, subjecting the digested DNA to an amplification means such as polymerase chain reaction (PCR) using primers comprising a nucleotide sequence capable of annealing to a non-cleaved form of a HpaII cleavable nucleotide sequence and subjecting the products of the PCR to separation or other detection means relative to a control, said control comprising another sub-sample of the sample of genomic DNA not subjected to digestion by HpaII but subjected to an amplification reaction using the same primers as for the digested DNA sample and then subjecting the products to the amplification reaction to the separation or detection means wherein the presence of PCR products in enzyme digested and non-digested samples is indicative of a HpaII-digestion-resistant marker (Hr), the absence and presence of PCR products in enzyme digested and undigested samples, respectively, is indicative of a HpaII-digestion-sensitive marker (HS) and the presence and absence of PCR products in enzyme digested and undigested samples, respectively, is indicative of a HpaII-digestion-dependent marker (H^{d}) wherein these sites are proposed to comprise genes or intergenic regions which are then screened for the presence of eRNAs or receive sequences.

The present invention is further described by the following non-limiting Examples.

### EXAMPLE 1

### A Role for Introns and Other Non-Coding RNAs in Dynamical Gene-Gene Communication, Genetic Multi-Tasking and Systems Integration

Potential cellular control molecules enabling multi-tasking and system integration must be capable of specifically targeted interactions with other molecules, must be plentiful (as limited numbers impair connectivity and adaptation in real and evolutionary time), and must carry information about the dynamical state of cellular gene expression. These goals are most directly or economically achieved by spatially and temporally synchronizing control molecule production with gene expression. Most protein-coding genes of higher eukaryotes are mosaics containing one or more intervening sequences (introns) of generally high sequence complexity, which are spliced out during pre-mRNA processing to generate a nuclear population of intronic RNA with concentration profiles linked to that of the exons, which are reassembled during this process to form mRNA, and which are subsequently translated into protein. The numbers of protein coding genes do not increase exponentially in complex organisms and hence cannot provide large scale cellular connectivity (which does increase exponentially). The genomes of higher organisms are, nevertheless, much larger than those of single celled organisms, with the vast majority of this size increase (after accounting for variable amounts of repetitive DNA) occurring within intron sequences and other non-protein-coding RNAs. Introns, therefore, fulfil the essential conditions for system connectivity and multi-tasking—(i) multiple output in parallel with gene expression; (ii) large numbers, especially if, as is likely (see below), they are further processed to smaller molecules after excision from the primary transcript; and (iii) the potential for specifically targeted interactions as a function of their sequence complexity. Sequences of just 20-30 nucleotides should generally have sufficient specificity for homology-dependent or structure-specific interactions. Introns are, therefore, excellent candidates for, and perhaps the only source of, possible control molecules for multi-tasking eukaryotic molecular networks, which relieve the problems associated with protein-based systems as genetic output can be multiplexed and target specificity can be efficiently encoded, assuming a receptive infrastructure.

### EXAMPLE 2

### Introns have Populated the Eukaryotic Lineage Late in Evolution

Modern nuclear introns are not ancient remnants of the prebiotic assembly of genes but the evolutionary descendants of self catalytic group II introns, which have similar splicing mechanisms (Lambowitz et al., Annu. Rev. Biochem. 62: 587-6221993; Eickbush, Nature 404: 940-941 2000). These elements appear to have penetrated the eukaryotic lineage late in evolution (Cavalier-Smith, Trends Genet. 7: 145-148 1991; Palmer et al., Curr. Opin. Genet. Dev. 1: 470-477, 1991; Mattick, Curr. Opin. Genet. Dev. 4: 823-831 1994; Stoltzfus et al., Science 265: 202-207 1994; Cho and Doolittle, J. Mol. Evol. 44: 573-584 1997; Wolf et al., J. Theor. Biol. 195: 167-186 1998) and to have expanded initially by retrotransposition (Cousineau et al., 2000; Eickbush, 2000) and later (after their sequence constraints were reduced by the evolution of the spliceosome) by other mutational, recombinational and insertional processes (Tarrio et al., Proc. Natl. Acad. Sci. USA 95: 1658-1662 1998). Self-catalytic group II introns do occur in bacteria, usually in tRNA genes (Ferat et al., Nature 364: 358-361 1993; Martinez-Abarca et al., Mol. Microbiol. 38: 917-926 2000) and the likely reason that introns are generally absent from prokaryotic protein coding sequences is the intimate coupling of transcription and translation in these cells, which does not allow time for intron excision (Mattick, Curr. Opin. Genet. Dev. 4: 823-831 1994).

The evolution of the nucleus and the separation of transcription and translation in the eukaryotes provided the opportunity for these introns to invade protein coding genes, as long as their removal by self splicing was efficient enough not to interfere with mRNA and protein production. The subsequent evolution of the spliceosome (involving the devolution of internal cis-acting catalytic RNAs into trans-acting spliceosomal RNAs and recruitment of accessory proteins) (Lambowitz et al. Annu. Rev. Biochem. 62: 587-622, 1993; Mattick, Curr. Opin. Genet. Dev. 4: 823-831 1994; Newman, Curr. Opin. Genet. Dev. 4: 298-304 1994; Stoltzfus, J. Mol. Evol. 49: 169-181 1999; Yean et al., Nature 408: 881-884 2000) made intron processing easier, which reduced the negative selection against them and allowed them more latitude. It also relaxed their internal sequence requirements, leaving them free to evolve and to explore new evolutionary space, based on RNA molecules produced in parallel with protein coding sequences (Mattick, Curr. Opin. Genet. Dev. 4: 823-831 1994). This would have been accelerated by the co-evolution of receptor systems for these molecules, involving RNA-protein, RNA-RNA and RNA-DNA/chromatin interactions, in the same way as other complex systems such as the ribosome and the spliceosome have evolved (Stoltzfus, J. Mol. Evol. 49: 169-181 1999). It is proposed, therefore, that intron-derived RNAs may have evolved trans-acting functions.

### EXAMPLE 3

### Intron Density Correlates with Developmental Complexity

Intron size and sequence complexity correlates well with developmental complexity, and introns comprise the majority of pre-mRNA sequences in the higher organisms. In developmentally simple eukaryotes like Schizosaccharomyces pombe, Aspergillus and Dictyostelium, introns comprise only 10-20% of the primary transcript, and are generally small with an average length of less than 100 bases and density about 1-3 introns per kilobase of protein coding sequence. These data are consistent with hybridization kinetic analyses of the relative sequence complexity of hnRNA (“heterogeneous nuclear RNA”) versus mRNA in lower eukaryotes (Davidson, 1976). In the higher plants there are 2-4 introns per gene of average length about 250 bases comprising about 50% of the primary transcript. In animals the average intron size rises to about 500 bases in Drosophila and C. elegans, and to about 3400 in human (6-7 introns per gene, average over 95% of the primary transcript) (Palmer et al., Curr. Opin. Genet. Dev. 1: 470-477, 1991; Deutsch et al. Nucleic Acids Res. 27: 3219-3228, 1999; Consortium, Nature 409: 860-921 2001; Venter et al., Science 291: 1304-1351 2001).

### EXAMPLE 4

### Introns have the Signatures of Information

Introns (and other non-protein coding RNAs, see below) of higher organisms exhibit all the signatures of information. They generally have high sequence complexity (Tautz et al., Nature 322: 652-656 1986) although one must distinguish between introns that may have evolved function and those that have not (which will be more degenerate) and take account of the differing proportions of functional and non-functional introns in lineages of different developmental complexity. While introns generally show less conservation than adjacent protein coding sequences, which are subject to strong constraints, so also do adjacent promoters and 5′ and 3′ untranslated regions of mRNA. The plasticity and more rapid evolution of these regulatory sequences does not mean they are non-functional and the present inventors suggest the same holds, in general, for introns.

### EXAMPLE 5

### Non-Coding RNAs Comprise the Majority of Genomic Output

Many (if not most, see below) transcripts from the genomes of higher organisms do not encode proteins at all (Eddy, Curr. Opin. Genet. Dev. 9: 695-699 1999; Erdmann et al., Nucleic Acids Res. 27: 192-195 1999). Where they have been examined these non-protein-coding transcripts are conserved and clearly functional. Well documented examples include XIST (involved in female X chromosome inactivation) (Brockdorff, Curr. Opin. GEnet. Dev. 8: 328-333 1998; Lee et al., Cell 75: 843-854 1999; Hong et al., Mamm, Genome 11: 220-224 2000) and H19 (mutants of which promote tumor development) (Wrana, Bioessays 16: 89-90 1994; Hurst et al. Trends Genet. 15: 134-135, 1999), both of which are imprinted and differentially spliced without encoding any protein. Others include roX1 and roX2 RNAs involved in dosage response (male X-chromosome activation) in Drosophila, heat shock response RNA in Drosophila, oxidative stress response RNAs in mammals, His-1 RNA involved in viral response/carcinogenesis in human and mouse, SCA8 RNA involved in spinocerebellar ataxia type 8 which is antisense to an actin-binding protein, and ENOD40 RNA in legumes and other plants (Eddy, Curr. Opin. Genet. Dev. 9: 695-699 1999; Erdmann et al., Nucleic Acids Res. 27: 192-195 1999; Nemes et al., Hum. Mol. Genet. 9: 1543-1551 2000). The 200 kb bithorax-abdominal A/B locus of Drosophila produces seven major transcripts (there may be minor ones as well), only three of which encode proteins, but all of which have phenotypic signatures and are developmentally regulated (Akam et al., Quant. Biol. 50: 195-200 1985; Hogness et al., Quant. Biol. 50: 181-194 1985; Lipshitz et al., Genes Dev. 1: 307-322 1987; Sanchez-Herrero et al., Drosophila. Development 107: 321-329 1989). These are not isolated examples. Many loci, including imprinted loci, express non-coding antisense and intergenic transcripts, some of which are alternatively spliced and developmentally regulated (Ashe et al., Genes Dev. 11: 2494-2509 1997; Lipman, Nucleic Acids Res. 25: 3580-3583 1997; Potter et al., Mamm. Genome 9: 799-806 1998; Lee et al., Nature Genet. 21: 400-404 1999; Filipowicz, Acta. Biochim. Pol. 46: 377-389 2000; Hastings et al., J. Biol. Chem. 275: 11507-11513 2000; Nemes et al., Hum. Mol. Genet. 9: 1543-1551 2000), as well as being stably detectable in the nucleus (Ashe et al., Genes Dev. 11: 2494-2509 1997).

### EXAMPLE 6

### Examples of Gene Regulation and Communication by Introns and Non-Coding RNAs

The activity of the heterochronic genes lin-14 and lin-41, which regulate developmental timing in C. elegans, are controlled by lin-4 and let-7 gene products encoding small RNAs that are antisense to repeated elements in the 3′ untranslated region of target mRNAs, and which appear to inhibit translation by RNA-RNA interactions (Lee et al., Cell 75: 843-854 1993; Wightman et al., C. elegans. Cell 75: 855-862 1993; Feinbaum et al., Caenorhabditis elegans. Dev. Biol. 210: 87-95 1999; Reinhart et al., Caenorhabditis elegans. Nature 403: 901-906 2000) possibly by targeting the mRNA for endoribonuclease attack (Nashimoto, FEBS Lett. 472: 179-186 2000). Lin-4 and let-7 do not contain obvious protein coding sequences, and the surrounding genomic sequences suggests that both are derived from functional introns surrounded by vestigial exons (Lee et al., Cell 75: 843-854 1993; Reinhart et al., Caenorhabditis elegans. Nature 403: 901-906 2000). Moreover, let-7 is functionally conserved in other bilaterian animals, from mollusks to mammals (Pasquinelli et al., Nature 408: 86-89 2000). Interestingly, the size of these RNAs (21-22 nt) is similar to that produced by the RNA interference (RNAi) pathway (Bass, Cell 101: 235-238 2000; Parrish et al., Mol. Cell. 6: 1077-1087 2000; Yang et al., Curr. Biol. 10: 1191-1200 2000; Zamore et al., Cell 101: 25-33 2000; Sharp, Genes Dev 15: 485-490 2001) (see below).

It has also been discovered that most small nucleolar RNAs (a group of more than 100 stable RNA molecules concentrated in the nucleolus) derive from processed introns of other genes, which encode various ribosomal proteins (e.g. L1, L5, L7, L13, S1, S3, S7, S8, S13 and others), ribosome-associated proteins (e.g. eIF-4A), nucleolar proteins (e.g. nucleolin, laminin, fibrillarin), the heat shock protein hsc70 and the cell-cycle regulated protein RCC1, among others (Prislei et al., Gene 163: 221-226 1993; Sollner-Webb, Cell 75: 403-405 1993; Bachellerie et al., Biochem. Cell. Biol. 73: 835-843 1995; Maxwell et al., Annu. Rev. Biochem. 64: 897-934, 1995; Nicoloso et al., J. Mol. Biol. 260: 178-195 1996; Rebane et al., Gene 210: 255-263 1998; Filipowicz et al., Acta. Biochim, Pol. 46: 377-389 1999; Filipowicz, Proc. Natl. Acad. Sci. USA 97: 14035-14037 2000). These provide both clear examples of dual gene outputs, and potential instances of coordinate regulation (efference control) involving intronic sequences, in this case of ribosomal biogenesis and cell growth (Pelczar et al., Mol. Cell. Biol. 18: 4509-4518 1998; Smith et al., Mol. Cell. Biol. 18: 6897-6909 1998; Tanaka et al., Genes Cells 5: 277-287 2000). More tellingly, some genes have so evolved that their protein coding capacity no longer exists, and their primary product is intron-derived small nucleolar RNAs (Tycowski et al., Nature 379: 464-466 1996; Bortolin et al., RNA 4: 445-454 1998; Pelczar et al., Mol. Cell. Biol. 18: 4509-4518 1998; Smith Smith et al., Mol. Cell. Biol. 18: 6897-6909 1998; Tanaka et al., Genes Cells 5: 277-287 2000) leading to the statement that “genes generating functionally important RNAs exclusively from their intron regions are probably more frequent than has been anticipated” (Bortolin et al., RNA 4: 445-454 1998).

These nucleolar RNAs are processed from introns by specific mechanisms involving endonucleolytic cleavage by double stranded RNase III-related enzymes (Caffarelli et al., X laevis. Biochem. Biophys. Res. Commun. 233: 514-517 1997; Chanfreau et al., EMBO J. 17: 3726-3737 1998; Qu et al., Mol. Cell. Biol. 19: 1144-1158 1999) (also implicated in RNAi, transgene silencing and methylation (Mette et al., EMBO J. 19: 5194-5201 2000)—see below), exonucleolytic trimming (Cecconi et al., Nucleic Acids Res. 23: 4670-4676 1995; Mitchell et al., Nature Struct. Biol. 7: 843-8461997; Allmang et al., EMBO J. 18: 5399-5410 1999a; Allmang et al., Genes Dev. 13: 2148-2158 1999b; van Hoof et al., Cell 99: 347-350 1999; van Hoof et al., EMBO J. 19: 1357-1365 2000) and possibly even adjacent RNA sequences that have self cleaving activity (Prislei et al., Gene 163: 221-226 1995). This processing occurs in large RNA processing complexes called exosomes, which are also involved in processing rRNA and small nuclear RNAs, and which contain at least 10 3′-5′ exonucleases, helicases and RNA binding proteins and which are found in both the nucleus and the cytoplasm (Mitchell, et al., Cell 91: 457-466 1997; Allmang et al., EMBO J. 18: 5399-5410 1999a,b; van Hoof et al. Cell 99: 347-350, 1999; Mitchell et al., Nature Struct. Biol. 7: 843-846 2000).

### EXAMPLE 7

### Intron Processing, Stability, Decay and Memory

After splicing, introns (initially in lariat form) are debranched (Ruskin et al., Science 229: 135-140 1985), a process that is itself subject to regulation (Ruskin et al., Science 229: 135-140 1985; Qian et al., Nucleic Acids Res. 20: 5345-5350 1992), but subsequent events are unknown. The inventors suggest that it is likely that excised introns are processed by specific pathways similar to those used to produce small nucleolar RNAs, and which generate multiple smaller species which can function independently as transacting signals in the network, affecting the metabolism of other RNAs and the modulation of chromatin structure, among other things (see below).

There are other documented examples of small transacting functional RNAs processed from longer transcripts (Sit et al., Science 281: 829-832 1998; Cavaille et al., Proc. Natl. Acad. Sci. USA 97: 14311-14316 2000). There are also large numbers of ribonucleases and other RNA-related proteins in plants and animals (see below), most of whose functions and substrates are not well defined. Such processing may also involve other splicing pathways (Santoro et al., Mol. Cell. Biol. 14: 6975-6982 1994; Kreivi et al., Curr. Biol. 6: 802-805 1996) and guide RNAs, possibly derived from introns or other non-protein-coding RNAs. These have been described as “riboregulators” (in relation to antisense RNAs) (Delihas, Mol. Microbiol. 15: 411-414 1995) and the “ribotype” (in relation to alternatively spliced mRNAs) (Herbert et al., Nature Genet. 21: 265-269 1999a), and may be considered to be part of the “soft wiring” of the cell (Herbert et al., Acad. Sci. 870: 119-132 1999b; Mattick, Curr. Opin. Genet. Dev. 4: 823-831 1994).

The decay characteristics of eRNAs are likely to be important to their function. Both short- and long-lived eRNAs provide a molecular memory of prior gene activation status, a significant efficiency gain over using bistable regulated gene networks as memories (Gardner et al., Escherichia coli. Nature 403: 339-342 2000). Differential eRNA decay (Qian et al., Nucleic Cids Res. 20: 5345-5350 1992) and diffusion rates would create spatially and temporally complex signal pulses that enable specific communication speeds, half lives and maximal communication radii for eRNA information transfer, allowing fine control of cellular activities.

### EXAMPLE 8

### Transvection and Chromatic Structure

The inventors propose predict that if eRNAs do have an important function in regulating gene expression, there should be genetic clues from intensively studied systems. A good candidate is the Drosophila bithorax complex, which is the archetypal developmental control locus, and which has been subjected to a considerable amount of genetic and molecular scrutiny. The bithorax region of this complex locus covers over 100 kb and contains 3 transcription units, one of which (Ubx) contains large introns and is differentially spliced to produce several variants of the morphogenetic homeobox protein UBX (Hogness et al., Quant. Biol. 50: 181-194 1985; Duncan, Annu. Rev. Genet. 21: 285-319 1987). The others are located upstream and are referred to as the early and late bxd units, and do not appear to encode proteins. Mutants of this locus can be classified into Ubx alleles, which disrupt the protein coding sequence and the abx, bx, pbx, and bxd alleles, which are located either within the introns of the Ubx unit (abx, bx) or in the 40 kb upstream region (pbx, bxd) and which affect the spatial pattern of UBX expression. The latter alleles are thought to represent cis-acting regulatory sequences controlling Ubx expression and are usually interpreted in terms of conventional enhancer elements, despite the fact that they are themselves transcribed. The bxd transcription unit produces a 27 kb transcript early in embryogenesis, which has a number of large introns, and is subject to differential splicing to give various small (˜1.2 kb) polyA+RNAs which do not contain any significant open reading frame (Akam et al., Quant. Biol. 50: 195-200 1985; Hogness et al., Quant. Biol. 50: 181-194 1985; Lipshitz et al., Genes. Dev. 1: 307-322 1987). The expression of this transcript is highly regulated during embryogenesis, in a pattern that is partially reflexive of Ubx transcript (Akam et al., Quant. Biol. 50: 195-200 1985; Irish et al., EMBO J. 8: 1527-1537 1989). A number of bxd insertional mutations have no effect on the amount or the size of the bxd polyA+RNA, suggesting that this species is irrelevant to the observed phenotypes and that the real import of the transcription and processing of this gene is to produce intronic RNAs (Hogness et al., Quant. Biol. 50: 181-194 1985). The “cis-regulatory” elements in this region also appear to be able to regulate the expression of Ubx in trans, since defective elements can be complemented by wild-type sequences on the other chromosome.

This phenomenon (partial complementation, or “allelic cross-talk”, between a mutation in a “cis-regulator” on one chromosome and one in the coding region of the adjacent gene on the other chromosome) has been known for many years, and is termed “transvection” (Judd, Cell 53: 841-843 1988; Pirrotta, Bioessays 12: 409-414 1990). Transvection has been observed in a number of different loci, and appears to be synapsis-dependent, since translocation of the “regulatory” sequences to other chromosomal sites normally diminishes or eliminates this trans-complementation of gene expression patterns (Judd, Cell 53: 841-843 1988; Pirrotta, Bioessays 12: 409-414 1990; Wu et al., Curr. Opin. Genet. Dev. 9: 237-246 1999). Mechanistically this has been interpreted in terms of enhancer elements from one copy of the gene being able to interact directly with its homolog on the other chromosome (i.e. to influence both promoters) because of their close alignment (Geyer et al., Drosophila. EMBO J. 9: 2247-2256 1990), although there are other propositions, mostly based on the same theme of chromosome pairing (Wu et al., Curr. Opin. Genet. Dev. 9: 237-246 1999). However, translocation of these regulatory sequences can in fact lead to a spectrum of transvection effects, ranging from weak to strong, suggesting that remote action is possible (Micol et al., Genetics 126: 365-373 1990) and that a simple model of chromosome pairing and transcriptional crossover is incorrect (Goldsborough et al., Nature 381: 807-810 1996). Moreover, these effects may be simply interpreted by regarding the “cis-acting regulatory regions” as encoding separate (non-coding RNA) genes.

Transvection at distance is accentuated in the presence of mutant alleles of the Polycomb gene (which normally acts to maintain repression of transcription of Ubx and other genes in cells where it was not initially activated) and at many loci is dependent on the zeste gene product, which acts in opposition to polycomb-group proteins to enhance transcription (Wu et al., Trends Genet. 5: 189-194 1989; Laney et al, Genes Dev. 6: 1531-1541 1992; Pirrotta, Biochim. Biophys. Acta 1424: M1-8 1999), indicating that factors other than chromosome pairing are involved in this process (Castelli-Gair et al., EMBO J. 9: 4267-4275 1990; Castelli-Gair et al., Genetics 126: 177-184 1990). Zeste null mutants do not affect chromosome pairing, even though transvection at some loci is entirely dependent on zeste (Gemkow et al., Drosophila melanogaster. Development 125: 4541-4552 1998; Pirrotta, Biochim. Biophys. Acta 1424: M1-8 1999). Moreover it has been shown that a region in the vicinity of the late bxd transcript which can attenuate Ubx expression can exert its action independent of its position (Castelli-Gair et al., Development 114: 877-184 1992a; Castelli-Gair et al., Mol. Gen. Genet. 234: 117-184 1992b). To explain such observations one has either to invoke DNA looping over enormous (interchromosomal) distances to bring regulatory proteins into contact with the Ubx promoter, or a (diffusible) substance expressed from these sequences, i.e. RNA.

Similar observations have been made at the downstream abdA-AbdB region of the bithorax complex which also encode homeotic proteins controlling segment identity. As in the case of bithorax itself, the sequences upstream of abdA and AbdB, which are referred to as the infrabdominal (iab) region, are thought to function as cis-acting regulatory elements, despite the fact that this region, like bxd, is also itself transcribed. Transvection (involving iab and abdA/AbdB alleles) at this locus is synapsis (pairing) independent and relatively insensitive to location, again suggesting that a trans-acting RNA may be involved (Hendrickson et al., Drosophila melangaster, Genetics 139: 835-848 1995; Hopmann et al., Genetics 139: 815-833 1995; Sipos et al., Genetics 149: 1031-1050 1998). The efficiency of this transvection is also different in different tissues, indicating that the state of differentiation has an effect on this process (Sipos et al., Genetics 149: 1031-1050 1998). Another (small, 800 bp) “element” in this region (Mcp) has also been shown to be capable of “trans-silencing”, independent of homology or homology pairing in the immediate vicinity of Mcp transgene inserts. The inventors propose that Mcp encodes a trans-acting RNA, whose ability to communicate with its target loci is affected by spatial separation and by polycomb/zeste mediated effects on chromatin architecture.

These genetic phenomena are connected, with common features being non-protein-coding RNAs and dynamic interactions and remodeling of chromatin involving DNA methylation and trithorax- and polycomb-group proteins, occurring in large complexes with a variety of other proteins, including histone modifying factors and transcription factors. The influence on transvection and other phenomena of complexes containing trithorax- and polycomb-group proteins may, therefore, be interpreted more easily in terms of maintaining, enhancing or inhibiting accessibility of these sites to trans-acting RNAs and/or executing signals from such RNAs.

### EXAMPLE 9

### Genetic Programming and the Evolution of Complex Organisms

The evolution of complex phenotypes is usually understood to proceed by a sequence from cells that were entirely unregulated and whose dynamics were governed by rate processes and input constraints. The existence of these cells provided the preconditions for the appearance of regulatory mechanisms which fine tuned rate processes. The inventors propose that these regulated networks, following a change in gene structure and output in the eukaryotic lineage, provided the necessary precondition for the appearance of controlled multi-tasked networks, which in turn, led to the appearance of programmed response networks capable of implementing stored sequences of dynamical activities in response to internal and external stimuli. Further, the inventors suggest that there is only one plausible mechanism for the evolution and control of multi-tasking in cell and developmental biology and that far from being evolutionary junk, nuclear introns and other non-protein-coding RNAs have evolved this function.

The majority of information in a multi-tasked network is held in control sequences. Non-protein-coding RNAs comprise the majority of the genomic output and unique sequence information in the higher eukaryotes and the evidence is growing that these RNAs are functional, as is the realization that RNA metabolism in these organisms is much more complex than previously realized.

The three critical steps in the evolution of this system were (i) the entry of introns into protein coding genes in the eukaryotic lineage, (ii) the subsequent relaxation of internal sequence constraints by the evolution of the spliceosome and the exploration of new sequence space, and (iii) the co-evolution of processing and receiver mechanisms for transacting RNAs, which are not yet well characterized but which are likely to involve the dynamic modeling and re-modeling of chromatin and DNA, as well as RNA-RNA and RNA-protein interactions in other parts of the cell. Steps (ii) and (iii) probably occurred, at least initially, by constructive neutral evolution (Stoltzfus, 1999), involving biased variation, epistatic interactions and excess capacities underlying a complex series of steps giving rise to novel structures and operations, and later by molecular co-evolution (Dover et al., Biol. Sci. 312: 275-289 1986). Once this system of RNA communication began to be established, the rate of evolution of functional introns would have accelerated (by positive selection), and led also to the evolution of other non-protein-coding RNAs, which are also usually spliced and are probably derived from genes that had lost their protein coding capacity, as appears to have occurred in the case of transcripts producing small nucleolar RNAs.

In practical terms then, the inventors propose that functional introns provide a cellular memory of recent transcriptional events and underpin a multiple output parallel processing system where gene activity at one locus can connect to others in real time, allowing integration and multi-tasking of a sophisticated network of cellular activity. In this scheme, non-protein-coding RNAs are control molecules in the network that do not require concomitant production of protein. Thus, there are two levels of information produced by gene expression in the higher organisms—mRNA and eRNA—allowing the concomitant expression of both structural (i.e. protein-coding) and networking information, the latter involving multiplex contacts between different genes and gene products via RNA signals that are implicit in primary transcripts. As some genes have evolved to express only eRNA and some genes lack introns, there are three types of genes in the higher organisms—those that encode only protein (which are rare), those that encode only eRNA, and those that encode both.

One prediction of this model is that many core proteins in the higher eukaryotes will be multi-tasked, i.e. have different roles in different sub-networks to produce different phenotypic outcomes. This appears to occur. For example, it has been shown that glycogen synthase kinase-3β participates both in the specification of the vertebrate embryonic dorsoventral axis (via the Wnt/wingless signaling pathway) and in the NF-ηB-mediated cell survival response following TNF activation (Hoeflich et al., Nature 406: 86-90 2000). Both cytochrome c and a flavoprotein (apoptosis-inducing factor) have redox functions in mitochondria as well as specific apoptogenic functions (Chinnaiyan, Neoplasia 1: 5-15 1999; Daugas et al., FEBS Lett. 476: 118-123 2000; Loeffler et al., Exp. Cell Res. 256: 19-26 2000). The XPD gene product functions in both transcription and excision repair of DNA (Lehmann, Genes Dev. 15: 15-23 2001). There are many other documented examples of proteins that participate in more than one developmental and signalling pathway (sub-network) (see e.g. Boutros et al., Mech. Dev. 83: 27-37 1999; Szebenyi et al., Int. Rev. Cytol. 185: 45-106 1999; Coffey et al., J. Neurosci. 20: 7602-7613 2000; O'Brien et al., Proc. Natl. Acad. Sci. USA 97: 12074-12078 2000). There are also examples of proteins having different, even antagonistic, functions in different settings, often as a result of alternative splicing (Jiang et al., Proc. Soc. Exp. Biol. Med. 220: 64-72 1999; Lopez, Annu. Rev. Genet. 32: 279-305 1998; Hastings et al., J. Biol. Chem. 275: 11507-11513 2000), a process that we predict will turn out to be regulated and guided not simply by tissue-specific RNA binding proteins/splicing factors but also by trans-acting RNAs produced by the activity of other genes (see, e.g. Hastings et al., J. Biol. Chem. 275: 11507-11513 2000). Consequently, developmental and phylogenetic profiling efforts will need to assign a range of biological, in addition to biochemical, functions to individual proteins and their splice variants in the network.

A multi-tasked network allows the rapid exploration of exponentially many protein expression profiles without equivalent increase in the size of the controlled parent network. The model therefore also predicts that the core proteome will be relatively stable in the higher organisms, which appears to be the case (Duboule et al., Trends Genet. 14: 54-59 1998; Rubin et al., Science 287: 2204-2215 2000) and that phenotypic variation will result primarily and quite easily from variation in the control architecture, rather than duplication and mutation of gene sub-networks. Once in place, therefore, a controlled multitasked network enables not only the efficient programming of different cellular phenotypes in the differentiation and development of multicellular organisms, but also rapid evolutionary radiation during expansions into uncontested environments, such as initially observed in the Cambrian explosion and as seen after major extinction events.

The corollary is that prokaryotes and simpler eukaryotes operating on simple protein control circuitry are limited in their phenotypic range, genome size and complexity not by the available diversity of polypeptide structures and chemistry, but by a primitive genetic operating system incapable of supporting integrated multi-tasking of gene networks. This would also explain why the Earth was restricted to simpler unicellular and colonial life forms for over 3 billion years, and the rapid evolution of complex life forms after the conditions for feasible parallel outputs were satisfied by the entry of introns into the eukaryotic lineage around 1.2 billion years ago, and the subsequent evolution of the necessary infrastructure for sending and receiving intronic and other non-protein-coding RNA signals.

Genomes are datasets with controls. The present invention examines, therefore, biology and genomes from the viewpoint of information and network theory and unifies a wide range of evolutionary and molecular genetic observations, including the long lag then sudden appearance of developmentally sophisticated multicellular organisms, the plasticity of phenotypic diversity despite the relative conservation of the core proteome and a wide range of unexplained molecular genetic phenomena that all intersect with RNA, the enabling molecule.

### EXAMPLE 10

### eRNA Regulators of HOX, ets-Domain Transcription Factor and Immunoglobulin Gene Expression

A method to identify eRNA elements and potential eRNA elements and/or their targets has been developed. The method searches the database of choice for known and predicted introns. The sequences of the known and predicted introns may then be compared in a BlastN search to identify from the non-redundant genome databases genes that are homologous to eRNA elements. eRNA elements may be embedded within introns or other non-coding RNA such as a 3′ or 5′ untranslated region (UTR). The method may also be used to screen such non-coding RNA sequences for eRNA elements. Short regions of homology between 19 and 200 nucleotides are considered significant to detect eRNA as it is known that short homologous regions of approximately 21 nucleotides act to modulate gene expression. The subject method identifies homologous sequences or complementary sequences which may be eRNA or target sequences.

A predicted intron sequence derived from chr19:38234-167860 is used in a BlastN search of the non-redundant human genome database to identify potential eRNA elements. The search reveals that this intron sequence comprise a number of candidate eRNA elements which may be directed to the regulation of multiple genes. eRNA elements are identified within introns by searching other parts of the genome, including protein- and non-protein-encoding regions, for homology with a candidate eRNA sequence. eRNA elements from this intron are proposed to be involved in regulation of activity of the ets-domain transcription factor, the human chloride channel transporter gene and the developmentally regulated HOX gene. This intron potentially contains an eRNA element directed to the regulation of immunoglobulin gene expression and an eRNA element directed to the regulation of expression of the gene encoding the nuclear factor of κ light polypeptide enhancer (NFκB1).

Predicted intron derived from chr19 between nucleotide sequences 38234-167860:

gtaggtggggaaggggtgtcaggtgggtactgcagatgggctctaggacctcggccttcaag
ttgtgtctgcccgcctcttgctactgtcttggatattttaaagtccttttgacgttgttctg
atttctgggcaggggacagagtaagtgtgtatttgctctgagactgttaatttggtatttcc
atcccaagttacagggaagacctcaggctgcaggttcctagctccgggctgaggtggcttgt
ggaggcagacagctgttgtctggaagtgcagagggctgggggctggccaggctgttactgag
ttcagaataggaggaaagagtgtgtagcaaagtcggcgctccttggccactgccagcattca
gagttgtcttgtttgccttgccttaaacgttgccttcctggacgcctacaaagtcaggttgt
aaccgctggccactgctgtgctcactggcagcccctgatttacgtgaggacctcaagtgtgt
gttgggcagaattccccagcgcttcccgtacaccccnccacccccagtgcagcatcgctcgg
tgcgtggctggtggactggaggagtgtgcgtgccggcagcactgccaggcacgtgcctaatg
ctctggccctgtgtgtttgtgttttcttcccgatttctgag [SEQ ID NO: 1]

| Predicted intron sequence from chr19 between nucleotide 38234-167860 | |
|---|---|
| comprises potential eRNA elements targeted to | |
| gi\|10280826\|gb\|AC012531.11\|AC012531 Homo sapiens, clone RP11-83K1, | |
| complete sequence | |
| | Length = 171949 |

| Score = 40.1 bits (20), Expect = 1.9 | |
|---|---|
| Identities = 20/20 (100%) | |
| Strand = Plus/Minus | |
| Query: 273 | agtgcagagggctgggggct 292 [SEQ ID NO: 2] |
| | \|\|\|\|\|\|\|\|\|\|\|\|\|\|\|\|\|\|\|\| |
| Sbjct: 168539 | agtgcagagggctgggggct 168520 [SEQ ID NO: 3] |
| Predicted intron sequence from chr19 between nucleotide 38234-167860 | |
| comprises potential eRNA elements targeted to | |
| gi\|2992476\|gb\|AC003666.1\|AC003666 Homo sapiens Xp22 BAC GS-551019 (Genome | |
| Systems Human BAC library) and | |
| | cosmids U199A7 and U209F2 (Lawrence Livermore X chromosome |
| | cosmid library) containing part of human chloride channel 4 |
| | gene, complete sequence |

Length = 151750

| Score = 40.1 bits (20), Expect = 1.9 | |
|---|---|
| Identities = 20/20 (100%) | |
| Strand = Plus/Plus | |
| Query: 264 | ttgtctggaagtgcagaggg 283 [SEQ ID NO: 4] |
| | \|\|\|\|\|\|\|\|\|\|\|\|\|\|\|\|\|\|\|\| |
| Sbjct: 102216 | ttgtctggaagtgcagaggg 102235 [SEQ ID NO: 5] |

| Predicted intron sequence from chr19 between nucleotide 38234-167860 | |
|---|---|
| comprises potential eRNA elements targeted to | |
| gi\|4689496\|gb\|AC006948.4\|AC006948 Homo sapiens chromosome 17, clone | |
| hRPK.334_M_10, complete sequence | |
| | Length = 168558 |

| Score = 40.1 bits (20), Expect = 1.9 | |
|---|---|
| Identities = 20/20 (100%) | |
| Strand = Plus/Minus | |
| Query: 563 | tggctggtggactggaggag 582 [SEQ ID NO: 6] |
| | \|\|\|\|\|\|\|\|\|\|\|\|\|\|\|\|\|\|\|\| |
| Sbjct: 20775 | tggctggtggactggaggag 20756 [SEQ ID NO: 7] |
| Predicted intron sequence from chr19 between nucleotide 38234-167860 | |
| comprises potential eRNA elements targeted to | |
| gi\|8894241\|emb\|AL157952.8\|AL157952 Human DNA sequence from clone RP5- | |
| 875K15 on chromosome 11p12-14.1 | |
| | Contains the gene for the eta-domain transcription factor |
| | EHF, ESTs, STSs and GSSs, complete sequence [Homo sapiens] |

Length = 114022

| Score = 40.1 bits (20), Expect = 1.9 | |
|---|---|
| Identities = 20/20 (100%) | |
| Strand = Plus/Plus | |
| Query: 243 | gcttgtggaggcagacagct 262 [SEQ ID NO: 8] |
| | \|\|\|\|\|\|\|\|\|\|\|\|\|\|\|\|\|\|\|\| |
| Sbjct: 64983 | gcttgtggaggcagacagct 65002 [SEQ ID NO: 9] |

| Predicted intron sequence from chr19 between nucleotide 38234-167860 | |
|---|---|
| comprises potential eRNA elements targeted to | |
| gi\|32387\|emb\|X61755.1\|HSHOX3D Human HOX3D gene for homeoprotein HOX3D | |
| | Length = 4968 |

| Score = 40.1 bits (20), Expect = 1.9 | |
|---|---|
| Identities = 20/20 (100%) | |
| Strand = Plus/Minus | |
| Query: 273 | agtgcagagggctgggggct 292 [SEQ ID NO: 10] |
| | \|\|\|\|\|\|\|\|\|\|\|\|\|\|\|\|\|\|\|\| |
| Sbjct: 166 | agtgcagagggctgggggct 147 [SEQ ID NO: 11] |

| Predicted intron sequence from chr19 between nucleotide 38234-167860 | |
|---|---|
| comprises potential eRNA elements targeted to | |
| >gi\|14718391\|gb\|AC021120.6\|AC021120 Homo sapiens clone RP11-34708, | |
| complete sequence | |
| | Length = 193980 |

| Score = 38.2 bits (19), Expect = 7.6 | |
|---|---|
| Identities = 19/19 (100%) | |
| Strand = Plus/Minus | |
| Query: 156 | tttgctctgagactgttaa 174 [SEQ ID NO: 12] |
| | \|\|\|\|\|\|\|\|\|\|\|\|\|\|\|\|\|\|\| |
| Sbjct: 131889 | tttgctctgagactgttaa 131871 [SEQ ID NO: 13] |

| Predicted intron sequence from chr19 between nucleotide 38234-167860 | |
|---|---|
| comprises potential eRNA elements targeted to | |
| gi\|2894631\|gb\|AC004152.1\|AC004152 Homo sapiens chromosome 19, fosmid | |
| 37308, complete sequence | |
| | Length = 37635 |

| Score = 38.2 bits (19), Expect = 7.6 | |
|---|---|
| Identities = 19/19 (100%) | |
| Strand = Plus/Minus | |
| Query: 280 | agggctgggggctggccag 298 [SEQ ID NO: 14] |
| | \|\|\|\|\|\|\|\|\|\|\|\|\|\|\|\|\|\|\| |
| Sbjct: 20673 | agggctgggggctggccag 20655 [SEQ ID NO: 15] |

| Predicted intron sequence from chr19 between nucleotide 38234-167860 | |
|---|---|
| comprises potential eRNA elements targeted to | |
| gi\|14091927\|gb\|AC025212.5\|AC025212 Homo sapiens chromosome 18, clone | |
| RP11-289A1, complete sequence | |
| | Length = 182258 |

| Score = 38.2 bits (19), Expect = 7.6 | |
|---|---|
| Identities = 19/19 (100%) | |
| Strand = Plus/Minus | |
| Query: 116 | gttgttctgatttctgggc 134 [SEQ ID NO: 16] |
| | \|\|\|\|\|\|\|\|\|\|\|\|\|\|\|\|\|\|\| |
| Sbjct: 51238 | gttgttctgatttctgggc 51220 [SEQ ID NO: 17] |
| Predicted intron sequence from chr19 between nucleotide 38234-167860 | |
| comprises potential eRNA elements targeted to | |
| gi\|13489123\|gb\|AC078776.12\|AC078776 Homo sapiens 12 BAC RP11-15519 | |
| (Roswell Park Cancer Institute Human BAC | |
| | Library) complete sequence |

Length = 95801

| Score = 38.2 bits (19), Expect = 7.6 | |
|---|---|
| Identities = 19/19 (100%) | |
| Strand = Plus/Plus | |
| Query: 630 | tgtgtgtttgtgttttctt 648 [SEQ ID NO: 18] |
| | \|\|\|\|\|\|\|\|\|\|\|\|\|\|\|\|\|\|\| |
| Sbjct: 58720 | tgtgtgtttgtgttttctt 58738 [SEQ ID NO: 19] |
| Predicted intron sequence from chr19 between nucleotide 38234-167860 | |
| comprises potential eRNA elements targeted to | |
| gi\|1302657\|gb\|U52112.1\|HSU52112 Homo sapiens Xq28 genomic DNA in the | |
| region of the L1CAM locus | |
| | containing the genes for neural cell adhesion molecule L1 |
| | (L1CAM), arginine-vasopressin receptor (AVPR2), C1 p115 |
| | (C1), ARD1 N-acetyltransferase related protein (TE2), |
| | renin-binding protein> |

Length = 174424

| Score = 38.2 bits (19), Expect = 7.6 | |
|---|---|
| Identities = 19/19 (100%) | |
| Strand = Plus/Minus | |
| Query: 278 | agagggctgggggctggcc 296 [SEQ ID NO: 20] |
| | \|\|\|\|\|\|\|\|\|\|\|\|\|\|\|\|\|\|\| |
| Sbjct: 73811 | agagggctgggggctggcc 73793 [SEQ ID NO: 21] |

| Predicted intron sequence from chr19 between nucleotide 38234-167860 | |
|---|---|
| comprises potential eRNA elements targeted to | |
| gi\|10567853\|gb\|AC035147.3\|AC035147 Homo sapiens chromosome 5 clone CTD- | |
| 2309M13, complete sequence | |
| | Length = 104939 |

| Score = 38.2 bits (19), Expect = 7.6 | |
|---|---|
| Identities = 22/23 (95%) | |
| Strand = Plus/Plus | |
| Query: 626 | gccctgtgtgtttgtgttttctt 648 [SEQ ID NO: 22] |
| | \|\|\|\|\|\|\|\|\|\|\|\|\|\|\| \|\|\|\|\|\|\| |
| Sbjct: 100838 | gccctgtgtgtttgtcttttctt 100860 [SEQ ID NO: 23] |

| Predicted intron sequence from chr19 between nucleotide 38234-167860 | |
|---|---|
| comprises potential eRNA elements targeted to | |
| gi\|9755473\|gb\|AC006452.4\|AC006452 Homo sapiens PAC clone RP4-592P3 from | |
| 7q31-q35, complete sequence | |
| | Length = 121703 |

| Score = 38.2 bits (19), Expect = 7.6 | |
|---|---|
| Identities = 19/19 (100%) | |
| Strand = Plus/Plus | |
| Query: 278 | agagggctgggggctggcc 296 [SEQ ID NO: 24] |
| | \|\|\|\|\|\|\|\|\|\|\|\|\|\|\|\|\|\|\| |
| Sbjct: 117068 | agagggctgggggctggcc 117086 [SEQ ID NO: 25] |

| Predicted intron sequence from chr19 between nucleotide 38234-167860 | |
|---|---|
| comprises potential eRNA elements targeted to | |
| gi\|9954648\|gb\|AC018758.2\|AC018758 Homo sapiens chromosome 19, BAC CTB- | |
| 6117 (BC52850), complete sequence | |
| | Length = 185409 |

| Score = 38.2 bits (19), Expect = 7.6 | |
|---|---|
| Identities = 19/19 (100%) | |
| Strand = Plus/Minus | |
| Query: 630 | tgtgtgtttgtgttttctt 648 [SEQ ID NO: 26] |
| | \|\|\|\|\|\|\|\|\|\|\|\|\|\|\|\|\|\|\| |
| Sbjct: 150073 | tgtgtgtttgtgttttctt 150055 [SEQ ID NO: 27] |

| Predicted intron sequence from chr19 between nucleotide 38234-167860 | |
|---|---|
| comprises potential eRNA elements targeted to | |
| gi\|9937750\|gb\|AC008750.7\|AC008750 Homo sapiens chromosome 19 clone CTD- | |
| 2616J11, complete sequence | |
| | Length = 143044 |

| Score = 38.2 bits (19), Expect = 7.6 | |
|---|---|
| Identities = 19/19 (100%) | |
| Strand = Plus/Minus | |
| Query: 464 | agcccctgatttacgtgag 482 [SEQ ID NO: 28] |
| | \|\|\|\|\|\|\|\|\|\|\|\|\|\|\|\|\|\|\| |
| Sbjct: 118714 | agcccctgatttacgtgag 118696 [SEQ ID NO: 29] |
| Predicted intron sequence from chr19 between nucleotide 38234-167860 | |
| comprises potential eRNA elements targeted to | |
| gi\|9506357\|gb\|M16230.2\|SUSSMP1 Strongylocentrotus purpuratus spicule | |
| matrix protein SM37, partial cds; | |
| | and spicule matrix protein SM50 precursor, gene, exon 1 |

Length = 14091

| Score = 38.2 bits (19), Expect = 7.6 | |
|---|---|
| Identities = 19/19 (100%) | |
| Strand = Plus/Plus | |
| Query: 631 | gtgtgtttgtgttttcttc 649 [SEQ ID NO: 30] |
| | \|\|\|\|\|\|\|\|\|\|\|\|\|\|\|\|\|\|\| |
| Sbjct: 14057 | gtgtgtttgtgttttcttc 14075 [SEQ ID NO: 31] |
| Predicted intron sequence from chr19 between nucleotide 38234-167860 | |
| comprises potential eRNA elements targeted to | |
| gi\|14596303\|emb\|AL356l57.14\|AL356157 Human DNA sequence from clone RP11- | |
| 733D4 on chromosome 10, complete | |
| | sequence [Homo sapiens] |

Length = 198917

| Score = 38.2 bits (19), Expect = 7.6 | |
|---|---|
| Identities = 19/19 (100%) | |
| Strand = Plus/Plus | |
| Query: 276 | gcagagggctgggggctgg 294 [SEQ ID NO: 32] |
| | \|\|\|\|\|\|\|\|\|\|\|\|\|\|\|\|\|\|\| |
| Sbjct: 86783 | gcagagggctgggggctgg 86801 [SEQ ID NO: 33] |
| Predicted intron sequence from chr19 between nucleotide 38234-167860 | |
| comprises potential eRNA elements targeted to | |
| gi\|14594822\|emb\|AJ314754.1\|APL314754 Anas platyrhynchos IgM gene | |
| (partial), mIgM gene (partial), IgA gene | |
| | (partial), mIgA gene (partial) and IgY gene (partial), |
| | clones 5.1, 13.1, 2.1 and PCR 00-106 |

Length = 48796

| Score = 38.2 bits (19), Expect = 7.6 | |
|---|---|
| Identities = 19/19 (100%) | |
| Strand = Plus/Plus | |
| Query: 404 | gccttcctggacgcctaca 422 [SEQ ID NO: 34] |
| | \|\|\|\|\|\|\|\|\|\|\|\|\|\|\|\|\|\|\| |
| Sbjct: 19162 | gccttcctggacgcctaca 19180 [SEQ ID NO: 35] |
| Predicted intron sequence from chr19 between nucleotide 38234-167860 | |
| comprises potential eRNA elements targeted to | |
| gi\|7012904\|gb\|AF213884.1\|AF213884S1 Homo sapiens nuclear factor of kappa | |
| light polypeptide gene enhancer in | |
| | B-cells 1 (NFKB1) gene, complete cds |

Length = 190000

| Score = 38.2 bits (19), Expect = 7.6 | |
|---|---|
| Identities = 19/19 (100%) | |
| Strand = Plus/Plus | |
| Query: 156 | tttgctctgagactgttaa 174 [SEQ ID NO: 36] |
| | \|\|\|\|\|\|\|\|\|\|\|\|\|\|\|\|\|\|\| |
| Sbjct: 92988 | tttgctctgagactgttaa 93006 [SEQ ID NO: 37] |

| Predicted intron sequence from chr19 between nucleotide 38234-167860 | |
|---|---|
| comprises potential eRNA elements targeted to | |
| gi\|2588626\|gb\|AC003081.1\|AC003081 Human BAC clone CTB-9H2 from 7q31, | |
| complete sequence [Homo sapiens] | |
| | Length = 149566 |

| Score = 38.2 bits (19), Expect = 7.6 | |
|---|---|
| Identities = 19/19 (100%) | |
| Strand = Plus/Plus | |
| Query: 395 | ttaaacgttgccttcctgg 413 [SEQ ID NO: 38] |
| | \|\|\|\|\|\|\|\|\|\|\|\|\|\|\|\|\|\|\| |
| Sbjct: 114135 | ttaaacgttgccttcctgg 114153 [SEQ ID NO: 39] |
| Predicted intron sequence from chr19 between nucleotide 38234-167860 | |
| comprises potential eRNA elements targeted to | |
| gi\|9187146\|emb\|AL133553.9\|AL133553 Human DNA sequence from clone GS1- | |
| 174L6 on chromosome 1 Contains part of | |
| | the gene for TPR (translocated promoter region (to |
| | activated MET oncogene)), a gene for a novel protein (MSF: |
| | megakaryocyte stimulating factor), ESTs, STSs and GSSs, |
| | complete sequ> |

Length = 190655

| Score = 38.2 bits (19), Expect = 7.6 | |
|---|---|
| Identities = 25/27 (92%) | |
| Strand = Plus/Plus | |
| Query: 126 | tttctgggcaggggacagagtaagtgt 152 [SEQ ID NO: 40] |
| | \|\|\|\|\|\|\|\| \|\|\|\|\|\|\|\|\|\|\|\|\| \|\|\|\| |
| Sbjct: 182695 | tttctgggtaggggacagagtatgtgt 182721 [SEQ ID NO: 41] |
| Predicted intron sequence from chr19 between nucleotide 38234-167860 | |
| comprises potential eRNA elements targeted | |
| gi\|6735496\|emb\|AL121925.10\|HSJ966J20 Human DNA sequence from clone RP5- | |
| 966J20 on chromosome 20 Contains | |
| | STSs and GSSs, complete sequence [Homo sapiens] |

Length = 39260

| Score = 38.2 bits (19), Expect = 7.6 | |
|---|---|
| Identities = 19/19 (100%) | |
| Strand = Plus/Plus | |
| Query: 505 | gaattccccagcgcttccc 523 [SEQ ID NO: 42] |
| | \|\|\|\|\|\|\|\|\|\|\|\|\|\|\|\|\|\|\| |
| Sbjct: 1220 | gaattccccagcgcttccc 1238 [SEQ ID NO: 43] |
| Predicted intron sequence from chr19 between nucleotide 38234-167860 | |
| comprises potential eRNA elements targeted to | |
| gi\|5123778\|emb\|AL035461.11\|HS967N21 Human DNA sequence from clone RP5- | |
| 967N21 on chromosome 20p12.3-13. | |
| | Contains the CHGB gene for chromogranin B (secretogranin |
| | 1, SCG1), a pseudogene similar to part of KIAA0172, the |
| | gene for a novel protein and KIAA1153, the gene for a |
| | novel MCM2/3/5 fam> |

Length = 139352

```
  Score = 38.2 bits (19), Expect = 7.6
  Identities = 19/19 (100%)
  Strand = Plus/Plus
```

### EXAMPLE 11

### eRNA Elements are Involved in the Regulation of Genes Expressed in Cancer

Jun Dimerization and TNFRSF6B Gene eRNA Element

A predicted intron sequence from chromosome 12 between nucleotide 156966-180225 is used in a BlastN search of the human genome database. The search identified eRNA elements residing in the intron with potential activities in the regulation of genes known to expressed in cancer.

A predicted intron residing on a fragment of DNA derived from chr12 between nucleotide sequences 156966-180225:—

| gtaagtgcccttccgggagctcacacccgctctctgtctcccctgtccttcctctgcttcat | |
|---|---|
| tttttcctggactctgaccgatgtttgcgttagagtatgtttgaacgtggggtcgattggga | |
| aggattaagccttggtgctgaggctggatattgcaggaggatacagggtgaatggagccggc | |
| ggggcggggcgggccgggctgctgtgccgtggctgctgttgtgctgacaccctctttcctag | |
| agaaacagcctcttattcacaaccagctgatttgaaatttcctgcag [SEQ ID NO: 44] | |
| Predicted intron sequence from chr12 between nucleotide 156966-180225 | |
| comprises potential eRNA elements targeted to | |
| gi\|14749255\|ref\|XM_034220.1\| Homo sapiens Jun dimerization protein | |
| p21SNFT (SNFT), mRNA | |
| | Length = 980 |

| Score = 44.1 bits (22), Expect = 0.053 | |
|---|---|
| Identities = 22/22 (100%) | |
| Strand = Plus/Plus | |
| Query: 184 | ggcggggcggggcgggccgggc 205 [SEQ ID NO: 45] |
| | \|\|\|\|\|\|\|\|\|\|\|\|\|\|\|\|\|\|\|\|\|\| |
| Sbjct: 186 | ggcggggcggggcgggccgggc 207 [SEQ ID NO: 46] |
| Predicted intron sequence from chr12 between nucleotide 156966-180225 | |
| comprises potential eRNA elements targeted to | |
| gi\|8246778\|emb\|AL121845.20\|HSJ583P15 Human DNA sequence from clone RP4- | |
| 583P15 on chromosome 20 Contains | |
| | ESTs, STSs, GSSs and ten CpG islands. Contains the |
| | TNFRSF6B gene for tumor necrosis factor receptor 6b |
| | (decoy), the 3′ part of the KIAA1088 gene, the ARFRP1 gene |
| | for ADP-ribosylation fa> |

Length = 120917

| Score = 44.1 bits (22), Expect = 0.053 | |
|---|---|
| Identities = 22/22 (100%) | |
| Strand = Plus/Plus | |
| Query: 184 | ggcggggcggggcgggccgggc 205 [SEQ ID NO: 47] |
| | \|\|\|\|\|\|\|\|\|\|\|\|\|\|\|\|\|\|\|\|\|\| |
| Sbjct: 43351 | ggcggggcggggcgggccgggc 43372 [SEQ ID NO: 48] |

| Predicted intron sequence from chr12 between nucleotide 156966-180225 | |
|---|---|
| comprises potential eRNA elements targeted to | |
| gi\|14523048\|ref\|NG_000006.1\| Homo sapiens genomic alpha globin region | |
| (HBA@) on chromosome 16 | |
| | Length = 43058 |

| Score = 42.1 bits (21), Expect = 0.21 | |
|---|---|
| Identities = 21/21 (100%) | |
| Strand = Plus/Plus | |
| Query: 185 | gcggggcggggcgggccgggc 205 [SEQ ID NO: 49] |
| | \|\|\|\|\|\|\|\|\|\|\|\|\|\|\|\|\|\|\|\|\| |
| Sbjct: 25749 | gcggggcggggcgggccgggc 25769 [SEQ ID NO: 50] |

| Score = 38.2 bits (19), Expect = 3.3 | |
|---|---|
| Identities = 22/23 (95%) | |
| Strand = Plus/Plus | |
| Predicted intron sequence from chr12 between nucleotide 156966-180225 | |
| comprises potential eRNA elements targeted to | |
| gi\|14336674\|gb\|AE006462.1\|AE006462 Homo sapiens 16p13.3 sequence section | |
| 1 of 8 | |
| | Length = 258002 |

```
  Score = 42.1 bits (21), Expect = 0.21
  Identities = 21/21 (100%)
  Strand = Plus/Plus
```

| | |
|---|---|
| Query: 185 | gcggggcggggcgggccgggc 205 [SEQ ID NO: 51] |
| \|\|\|\|\|\|\|\|\|\|\|\|\|\|\|\|\|\|\|\|\| | |
| Sbjct: 154885 | gcggggcggggcgggccgggc 154905 [SEQ ID NO: 52] |

```
  Score = 38.2 bits (19), Expect = 3.3
  Identities = 22/23 (95%)
  Strand = Plus/Plus
```

### EXAMPLE 12

### eRNA Elements Which Overlap and Which are Directed to the Regulation of Multiple Genes

A predicted intron sequence derived from chr12 between nucleotides: 156966-18022 is used in a BlastN search of the non-redundant human genome database to identify potential eRNA elements. The search reveals that a plurality of putative eRNA elements are embedded within a single intron and that a single eRNA element may perform regulatory functions directed at multiple genes. eRNA elements are identified within introns by searching other parts of the genome, including protein- and non-protein-encoding regions, for homology with a candidate eRNA sequence. eRNA elements from this intron are potentially involved in regulation of X-chromosome activity as well as several unannotated genes derived from human DNA.

Predicted intron sequence from chr12 between nucleotide 156966-180225:—

| gtatgtaccgtgctgggaccacttccccaggtgccttccccacccagccaggtctgtagttt | |
|---|---|
| tgaaagtcttgtatagctttttccttggtttaaaagcaataaatgcccactggagataaatt | |
| agaaaatatggaagaaagctataaaaaagaaactaaaaaaatctcttgtaattccaccactc | |
| aaatataactttttttcttaaaaaattttttttctcttacttagagacaggcagggtctggc | |
| tctgtcccccaggctggagtgcagtggtgccatcatagctcactgcagcctcaacctcttgg | |
| gctcaaggcattctctcgcctcagcctcctgagcagctgggactgcaggcatgagccatggt | |
| tcctgggcattttctcttgatattttgatgaagcagcctctttgtccccaggtcatagctgc | |
| ttaagacactatgtacagagatcttagttgaatgagacaagtgacttctggctgtgccctgc | |
| agataggccttgggtgcagccatggtttgtagattcccctggagaaatccaagcaacacaca | |
| tgtatttggtactcactaagtgcctacagaaccaaaccgaaactgggccgcactggggagga | |
| gatcaccgtggagaccggagggcgcactcacggagagt [SEQ ID NO: 53] | |
| Predicted intron sequence from chr12 between nucleotide 156966-180225 | |
| comprises potential eRNA elements targeted to: | |
| gi\|13162510\|gb\|AC011443.6\|AC011443 Homo sapiens chromosome 19 clone CTC- | |
| 218B8, complete sequence | |
| | Length = 156776 |

| Score = 151 bits (76) , Expect = 7e-34 | |
|---|---|
| Identities = 112/124 (90%) | |
| Strand = Plus/Minus | |
| Query: 238 | cagggtctggctctgtcccccaggctggagtgcagtggtgccatcatagctcactgcagc |
| 297 [SEQ ID NO: 54] | |
| | \|\|\|\|\|\|\|\| \|\|\|\|\|\|\| \|\|\|\|\|\|\|\|\|\| \|\|\|\|\|\|\|\|\| \|\| \|\|\|\|\| \|\|\|\|\|\|\|\|\|\|\|\| |
| Sbjct: 49308 | cagggtcttgctctgttgcccaggctggggtgcagtggcgcaatcatggctcactgcagc |
| 49249 [SEQ ID NO: 55] | |
| Query: 298 | ctcaacctcttgggctcaaggcattctctcgcctcagcctcctgagcagctgggactgca |

357 [SEQ ID NO: 56]

| | \|\|\|\|\|\|\|\|\| \|\|\|\|\|\|\|\|\|\| \|\|\| \|\|\| \|\|\|\|\|\|\|\|\|\|\|\|\|\|\|\|\|\|\|\|\|\|\|\|\|\|\|\| \|\| |
|---|---|
| Sbjct: 49248 | ctcaacctcctgggctcaagccatcctcccgcctcagcctcctgagcagctgggactaca |
| 49189 [SEQ ID NO: 57] | |
| Query: 358 | ggca 361 |
| | \|\|\|\| |
| Sbjct: 49188 ggca 49185 | |
| Score = 101 bits (51), Expect = 6e-19 | |
| Identities = 93/107 (86%) | |
| Strand = Plus/Minus | |
| Query: 247 | gctctgtcccccaggctggagtgcagtggtgccatcatagctcactgcagcctcaacctc |
| 306 [SEQ ID NO: 58] | |
| | \|\|\|\|\|\|\|\| \|\|\|\|\|\|\|\|\|\|\|\|\|\| \|\|\|\|\|\|\|\| \|\|\|\| \|\|\|\|\|\|\|\|\|\|\|\|\|\|\|\| \| \|\|\| |
| Sbjct: 81907 | gctctgtcacccaggctggagtgtagtggtgcaatcagagctcactgcagcctccaactc |
| 81848 [SEQ ID NO: 59] | |
| Query: 307 | ttgggctcaaggcattctctcgcctcagcctcctgagcagctgggac 353 |
| [SEQ ID NO: 60] | |
| | \|\|\|\|\|\|\|\|\|\| \|\| \|\|\| \| \|\|\|\|\|\|\|\|\|\|\|\|\|\|\| \|\|\|\| \|\|\|\| |
| Sbjct: 81847 | ctgggctcaagcaatcctcccacctcagcctcctgagtagctaggac 81801 |
| [SEQ ID NO: 61] | |
| Score = 101 bits (51), Expect = 6e-19 | |
| Identities = 105/123 (85%) | |
| Strand = Plus/Plus | |
| Query: 248 | ctctgtcccccaggctggagtgcagtggtgccatcatagctcactgcagcctcaacctct |
| 307 [SEQ ID NO: 62] | |
| | \|\|\|\|\|\|\| \|\|\|\|\|\|\|\|\|\|\|\|\|\|\|\|\|\|\|\|\|\|\| \|\|\| \| \|\|\|\|\|\|\|\|\|\| \|\|\|\| \|\|\|\| |
| Sbjct: 79220 | ctctgtcacccaggctggagtgcagtggtgcgatcttggctcactgcaacctccgcctcc |
| 79279 [SEQ ID NO: 63] | |
| Query: 308 | tgggctcaaggcattctctcgcctcagcctcctgagcagctgggactgcaggcatgagcc |
| 367 [SEQ ID NO: 64] | |
| | \|\|\|\| \|\|\|\|\| \|\|\|\|\|\| \|\|\|\|\|\|\|\|\|\|\|\| \|\|\| \|\|\|\|\|\|\|\|\|\| \|\|\|\|\| \|\| \|\|\| |
| Sbjct: 79280 | tgggttcaagtgattctcctgcctcagcctcccgagtagctgggactacaggcgtgtgcc |
| 79339 [SEQ ID NO: 65] | |
| Query: 368 | atg 370 |
| | \|\|\| |
| Sbjct: 79340 | atg 79342 |

| Predicted intron sequence from chr12 between nucleotide 156966-180225 | |
|---|---|
| comprises potential eRNA elements targeted to: | |
| gi\|6649930\|gb\|AF031075.1\|AF031075 Homo sapiens chromosome X, cosmid | |
| Qc8D3, complete sequence | |
| | Length = 44163 |

| Score = 1453 bits (733), Expect = 0.0 | |
|---|---|
| Identities = 747/754 (99%) | |
| Strand = Plus/Plus | |
| Query: 1 | gtggggacaaacagaaagacacaaggaacaattagaggctctccatagcaatgtcagaga |
| 60 [SEQ ID NO: 66] | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| Sbjct: 22925 | gtggggacaaacagaaagacacaaggaacaattagaggctctccatagcaatgtcagaga |
| 22984 [SEQ ID NO: 67] | |
| Query: 61 | tagggcagagcggatggtggtgacaacgctctgacaaacgttactattgaacgagagtca |
| 120 [SEQ ID NO: 68] | |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| Sbjct: 22985 | tagggcagagcggatggtggtgacaacgctctgacaaacgttactattgaacgagagtca |

| [SEQ ID NO: 69] | |
|---|---|
| Predicted intron sequence from chr12 between nucleotide 156966-180225 | |
| comprises potential eRNA elements targeted to | |
| gi\|4508111\|gb\|AC005072.2\|AC005072 Homo sapiens BAC clone CTB-181H17 from | |
| 7q21.2-q31.1, complete sequence | |
| | Length = 69367 |

| Score = 147 bits (74), Expect = 1e-32 | |
|---|---|
| Identities = 110/122 (90%) | |
| Strand = Plus/Plus | |
| Query: 238 | cagggtctggctctgtcccccaggctggagtgcagtggtgccatcatagctcactgcagc |
| 297 [SEQ ID NO: 70] | |
| | \|\|\|\|\|\|\|\| \|\|\|\|\|\|\|\| \|\|\|\|\|\|\|\|\|\|\|\|\| \|\|\|\|\|\|\|\|\| \|\|\|\|\|\|\|\|\|\|\|\|\|\|\|\|\|\| |
| Sbjct: 46265 | cagggtcttgctctgtcacccaggctggagttcagtggtgcaatcatagctcactgcagc |
| 46324 [SEQ ID NO: 71] | |
| Query: 298 | ctcaacctcttgggctcaaggcattctctcgcctcagcctcctgagcagctgggactgca |
| 357 [SEQ ID NO: 72] | |
| | \|\|\|\|\| \|\|\| \|\|\|\|\|\|\|\|\|\| \|\| \|\|\| \| \|\|\|\|\|\|\|\|\|\|\|\|\|\|\| \|\|\|\|\|\|\|\|\|\|\|\|\| |
| Sbjct: 46325 | ctcaaactcctgggctcaagcaatcctcccacctcagcctcctgagtagctgggactgca |
| 46384 [SEQ ID NO: 73] | |
| Query: 358 | gg 359 |
| | \|\| |
| Sbjct: 46385 gg 46386 | |
| Score = 93.7 bits (47), Expect = 1e-16 | |
| Identities = 86/99 (86%) | |
| Strand = Plus/Minus | |
| Predicted intron sequence from chr12 between nucleotide 156966-180225 | |
| comprises potential eRNA elements targeted to: | |
| gi\|13624997\|emb\|AL356214.20\|AL356214 Human DNA sequence from clone RP11- | |
| 30E16 on chromosome 10, complete | |
| | sequence [Homo sapiens] |

Length = 163964

| Score = 133 bits (67) , Expect = 2e-28 | |
|---|---|
| Identities = 106/119 (89%) | |
| Strand = Plus/Minus | |
| Query: 250 | |
| ctgtcccccaggctggagtgcagtggtgccatcatagctcactgcagcctcaacctcttg 309 | |
| [SEQ ID NO: 74] | |
| | \|\|\|\|\| \|\|\|\|\|\|\|\|\|\|\|\|\|\|\|\|\|\|\|\| \|\|\|\|\|\|\|\| \|\|\|\|\|\|\|\|\|\|\|\|\|\|\|\|\|\|\|\|\| |
| \|\| | |
| Sbjct: 115382 | |
| ctgtcacccaggctggagtgcagtggcgccatcatggctcactgcagcctcaacctcctg 115323 | |
| [SEQ ID NO: 75] | |
| Query: 310 | ggctcaaggcattctctcgcctcagcctcctgagcagctgggactgcaggcatgagcca |
| 368 [SEQ ID NO: 76] | |
| +TL,1 | \|\|\|\|\|\|\|\| \|\|\| \|\| \|\|\|\|\|\|\|\|\|\|\|\|\|\|\| \|\|\|\|\|\| \|\|\| \|\|\|\|\|\|\|\| \|\|\|\| |
| Sbjct: 115322 | ggctcaagccatcctaccacctcagcctcctgagtagctggaactacaggcatgggcca |
| 115264 [SEQ ID NO: 77] | |
| Score = 97.6 bits (49), Expect = 9e-18 | |
| Identities = 97/113 (85%) | |
| Strand = Plus/Minus | |
| Predicted intron sequence from chr12 between nucleotide 156966-180225 | |
| comprises potential eRNA elements targeted to: | |
| gi\|3165399\|gb\|AC003684.1\|AC003684 Homo sapiens Xp22 BAC GSHB-519E5 | |
| (Genome Systems Human BAC library) | |
| | complete sequence |

Length = 210954

| Score = 135 bits (68), Expect = 4e-29 | |
|---|---|
| Identities = 95/104 (91%) | |
| Strand = Plus/Plus | |
| Query: 241 | ggtctggctctgtcccccaggctggagtgcagtggtgccatcatagctcactgcagcctc |
| 300 [SEQ ID NO: 78] | |
| | \|\|\|\|\| \|\|\|\|\|\|\|\| \| \|\|\|\|\|\|\|\|\|\|\|\|\|\|\|\|\|\|\|\|\|\|\|\|\|\| \|\|\|\|\|\|\|\|\|\|\|\|\|\|\|\| |
| Sbjct: 46790 | ggtctcgctctgtcactcaggctggagtgcagtggtgccatcacagctcactgcagcctc |
| 46849 [SEQ ID NO: 79] | |
| Query: 301 | aacctcttgggctcaaggcattctctcgcctcagcctcctgagc 344 |
| [SEQ ID NO: 80] | |
| | \|\| \|\|\|\|\|\|\|\|\|\|\|\|\| \|\|\| \|\|\|\|\| \|\|\|\|\|\|\|\|\|\|\|\|\|\|\|\| |
| Sbjct: 46850 | aaattcttgggctcaagccatcctctcacctcagcctcctgagc 46893 |

```
[SEQ ID NO: 81]
  Score = 113 bits (57), Expect = 2e-22
  Identities = 99/113 (87%)
  Strand = Plus/Minus
```

### EXAMPLE 13

### Generic Methods for Determining the Effect of Putative eRNA

A protein-encoding gene (1), which comprises at least one intron suspected of encoding an eRNA, is modified to prevent translation of the encoded protein but to otherwise preserve transcription of the primary transcript.

A gene so modified (2) is conveniently prepared by oligonucleotide-directed (or site-directed) mutagenesis to convert the start codon (ATG) of the gene to a non-start codon (e.g., AAG or TAG) and to introduce a stop codon (e.g., TAG, TAA, TGA) closely downstream (e.g., within 30 bases) of the normal start codon. The site-directed mutagenesis involves hybridizing an oligonucleotide encoding the desired mutation to a template DNA, wherein the template is the single-stranded form of a plasmid or bacteriophage containing the unaltered or parent gene sequence. After hybridization, a DNA polymerase is used to synthesize an entire second complementary strand of the template that will thus incorporate the oligonucleotide primer and will code for the selected alteration in the parent gene sequence. The resultant heteroduplex molecule is then transformed into a suitable host cell, usually a prokaryote such as E. coli. After the cells are grown, they are plated onto agarose plates and screened using the oligonucleotide primer having a detectable label to identify the bacterial colonies having the mutated or modified gene.

The intron(s) of the parent and modified genes are removed by site-directed mutagenesis or by other standard techniques to provide (3) a modified gene encoding an intronless primary transcript from which a wild-type protein can be translated and (4) a modified gene encoding an intronless primary transcript from which a wild-type protein cannot translated.

Each of the above genes (1-4) is then inserted into a suitable expression vector and the construct so produced is transfected into cells. Expression of the inserted genes (1-4) in the transfected cells will result, respectively, in:—

(a) a normal primary transcript, including introns, from which a functional wild-type protein can be produced;

(b) a primary transcript, excluding introns, from which a functional wild-type protein can be produced;

(c) a primary transcript, including introns, from which a functional wild-type protein cannot be produced; and

(d) a primary transcript, excluding introns, from which a functional wild-type protein cannot be produced.

The phenotypic effects of (a)-(d) are then compared (e.g., by pairwise comparisons) to discriminate which effects may be ascribed to protein and which may be ascribed to eRNA.

Alternatively, genetic complementation to discriminate whether putative eRNA sequences are encoding genuine trans-acting RNAs or cis-acting transcription factor binding sites, can be assessed by allelic replacement with an intronless gene and determination of the phenotypic effect thereof, followed by complementation with the intron-containing gene which cannot produce a protein (e.g. because its translational start codon has ben rendered non-functional by site-directed mutation). If wild-type function is restored by the latter, the complementing genetic factor must be an eRNA derived from the intron. Appropriate secondary controls are employed to confirm whether a transcript is produced and spliced normally (e.g., using Northern blots) and whether a protein is or is not expressed (e.g., using Western blots) as appropriate to the particular construct.

### EXAMPLE 14

### Idenfication of eRNA Candidates in Meiotic Genes

A subset of nucleotide repeats in the S. cerevisiae genome is obtained and then filtered by taking intronic sequences of all known meiotic genes and removing all repeated sequences not in the sequences of the introns. This leaves a putative signal of an eRNA gene regulation network. In Table 2, the gene carrying an intron which is repeated is identified in the left hand column. The nucleotide sequence of the repeat intronic sequence is then shown in the penultimate left hand column.

These 16mer sequences are then screened for potential receiver sequences in 245,000 sequences in the genome. In Table 2, there are three types of putative receiver sequences which are located in two regions:

i) within a gene (third most right column); or

ii) in an intergenic region located:

a) upstream (second most right hand column); or

b) downstream (most right hand column).

Many of these genes are known to be involved in meiotic processes, including cell division. The chance that any given sequence of 16 nucleotides would occur accidently at more than one locus in the yeast genome is less than 1 in 100. The odds against an accidental finding that sequences from introns of genes involved in meoisis occur in or near a set of other genes involved in meiosis is astronomically small, and thus this network must be real. Consequently, this confirms that the identifier of potential eRNA and receiver sequences is a significant event, supporting the concept of eRNA networking. The role of any particular candidate eRNAs in the network may be determined and confirmed by analyses such as set out in Example 13.

TABLE 2

eRNA AND RECEIVE SEQUENCES IN SACCHAROMYCES CEREVISIAE
MEIOTIC GENES

| | | | | | |
|---|---|---|---|---|---|
| Intron | | | | | |
| Bearing Gene | SEQ ID No. | Repeat | Hit | Upstream | Downstream |

| | | | | | |
|---|---|---|---|---|---|
| AMA1 | 82 | CTTATTTTTTCATT | | RPL15A | YLR030W (119) |
| | | AT | | (581) | |
| | 83 | TTTTTCATTATGAA | PHA2 | | |
| | | AA | | | |
| | 84 | AAAATATTTGTTAG | CWH43 | | |
| | | TA | | | |
| DMC1 | 85 | CTGCTGTAGAGGTT | | RIM15 | YFL032W (332) |
| | | CT | | (113) | |
| | 86 | CTAATAATTTGGAA | YNL156C | | |
| | | AGGA | | | |
| | 87 | ATAACATTTTTAAA | | ATP3 (167) | FIG1 (291) |
| | | AC | | | |
| | | | SEC8 | | |
| | 88 | GGTTCTTTCCCCCT | | MNN4 (136) | YKT9 (671) |
| | | TT | | | |
| | 89 | CTAATAATTTGGAA | YNL156C | | |
| | | AGG | ARP8 | | |
| HFM1 | 90 | AAGTGGTTTTTCTG | YCR024C | | |
| | | GA | | | |
| | 91 | TAGATAATAAAAG | | PPA1 (112) | RPN1 (133) |
| | | AAA | | | |
| | 92 | CTAGATAATAAAA | | YPL141C | MKK2 (117) |
| | | GAA | | (1336) | |
| HOP2 | 93 | GTTAAGTATTTTTT | | HXT12 | YIL169C (273) |
| | | TA | | (2999) | YOL155C (102) |
| | | | | HXT11 | |
| | | | | (1625) | |
| MMS2 | 94 | CCTTTCAAAACTTA | | FIT1 (586) | YDR535C (1120) |
| | | TA | | | |
| | 95 | ATTTGTTAGTATAT | | MAM33 (8) | RPS24B (473) |
| | | GT | | | |
| PCH2 | 96 | TCTTTCTTTCCTTCT | | SGT1 (201) | ASE1 (114) |
| | | T | | | |
| | 97 | TATGTTTTTTTCTTT | YLR379W | | |
| | | T | | | |
| | 98 | TCTTCATAAAAAA | | YGL034C | HOP2 (165) |
| | | GCA | | (1881) | |
| | 99 | TTCTTTTTCTTTCTT | | NOG1 (144) | SSU1 (728) |
| | | TC | | | |
| | 100 | GTATGTTTTTTTCT | | YKL063C | MSN4 (807) |
| | | TT | | (903) | |
| | 101 | CTTTTTCTTTCTTTC | SPP41 | | |
| | | CTT | | | |
| | 102 | TTTTTTTCTTTTATT | YGL131C | | |
| | | CT | | | |
| | 103 | TTTTATTCTACTTTT | | TH(GUG)E1 | CHO1 (64) |
| | | A | | (152) | |
| RAD14 | 104 | AATTTAACGATGA | | NVJ1 (101) | UTP9 (118) |
| | | GATG | | | |
| | 105 | CAAACACAGAATC | YDL189W | | |
| | | ATTT | | | |
| | 106 | CGATGAGATGAGC | | URA7 (144) | MRPL16 (315) |
| | | TGTG | | | |
| SRC1 | 107 | TTTTTTTTGTTTTTG | | VPS25 (888) | URA8 (101) |
| | | A | | | |
| | 108 | TTAATTTTTTTTGA | YMR192W | | |
| | | AT | | | |
| | 109 | TAATTTTTTTTGAA | | SUL1 (333) | PCA1 (701) |
| | | TTT | | | |
| | 110 | TTTTTTTTGAATTTT | | BUR6 (38) | TR(ACG)E (356) |
| | | T | | YAP3 (220) | TV(AAC)H (18) |
| | | | | RPL34B | MMF1 (372) |
| | | | | (409) | |
| | 111 | TTTTTTTGAATTTTT | | VPS45 (429) | PAN2 (82) |
| | | T | | YAP3 (219) | TV(AAC)H (19) |
| | | | | YPR078C | MRL1 (332) |
| | | | | (273) | |
| | 112 | AGTTTTAATTTTTT | | MSC6 | GDS1 (354) |
| | | TT | | (1559) | |
| | 113 | TTTTTTTTTGTTTTT | SAP4 | | |
| | | G | | | |
| | 114 | TTTTTTTGTTTTTGA | | YHR032W | YHR033W (60) |
| | | TTT | | (399) | |
| | 115 | TTGAATTTTTTTTT | YOR154W | | |
| | | GT | | | |
| | 116 | TTTTAATTTTTTTTG | RAD59 | | |
| | | A | | | |
| | 117 | AATAAATTGTACTC | STT4 | | |
| | | AC | | | |
| | 118 | TTTTTGAATTTTTTT | | YAP3 (216) | TV(AAC)H (22) |
| | | TT | | YPR078C | MRL1 (335) |
| | | | | (270) | MCM1 (201) |
| | | | | ARG80 | |
| | | | | (534) | |
| | 119 | AAAATTCAAAAAA | | YAP3 (221) | TV(AAC)H (17) |
| | | AAT | | | |
| | 120 | AAAAAAATTCAAA | | YAP3 (218) | TV(AAC)H (20) |
| | | AAA | | YPR078C | MRL1 (333) |
| | | | | (272) | |
| YLR211C | 121 | TTTTTTTTTGTTCAT | | KGD1 (130) | AYR1 (341) |
| | | G | | | |

### EXAMPLE 15

### GRIA 3RNA Network

FIG. 6 provides and example of an eRNA network centred around the GRIA2, GRIA3 and GRIA4 genes which all share parts of an intronic sequence shown in the Figure. It is proposed that this intronic sequence is an eRNA.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Akam, M. E., A. Martinez-Arias, R. Weinzierl and C. D. Wilde. 1985. Function and expression of ultrabithorax in the Drosophila embryo. Cold Spring Harb. Symp., Quant. Biol. 50: 195-200.

Albert, R., H. Jeong and A. L. Barabasi. 2000. Error and attack tolerance of complex networks. Nature 406: 378-382.

Allmang, C., J. Kufel, G. Chanfreau, P. Mitchell, E. Petfalski and D. Tollervey. 1999a. Functions of the exosome in rRNA, snoRNA and snRNA synthesis. EMBO J. 18: 5399-5410.

Allmang, C., E. Petfalski, A. Podtelejnikov, M. Mann, D. Tollervey and P. Mitchell. 1999b. The yeast exosome and human PM-Scl are related complexes of 3′→5′ exonucleases. Genes Dev. 13: 2148-2158.

Altschul et al., 1997, Nucl. Acids Res. 25:3389.

Ausubel et al., “Current Protocols in Molecular Biology” John Wiley & Sons Inc, 1994-1998, Chapter 15.

Almeida, A. C., V. M. Fernandes de Lima and A. F. Infantosi. 1998. Mathematical model of the CA1 region of the rat hippocampus. Phys. Med. Biol. 43: 2631-2646.

Andersen, R. A., L. H. Snyder, D. C. Bradley and J. Xing. 1997. Multimodal representation of space in the posterior parietal cortex and its use in planning movements. Annu. Rev. Neurosci. 20: 303-330.

Ashe, H. L., J. Monks, M. Wijgerde, P. Fraser and N. J. Proudfoot. 1997. Intergenic transcription and transinduction of the human beta-globin locus. Genes Dev. 11: 2494-2509.

Bachellerie, J. P., M. Nicoloso, L. H. Qu, B. Michot, M. Caizergues-Ferrer, J. Cavaille and M. H. Renalier. 1995. Novel intron-encoded small nucleolar RNAs with long sequence complementarities to mature rRNAs involved in ribosome biogenesis. Biochem. Cell. Biol. 73: 835-843.

Bass, B. L. 2000. Double-stranded RNA as a template for gene silencing. Cell 101: 235-238.

Becskei, A. and L. Serrano. 2000. Engineering stability in gene networks by autoregulation. Nature 405: 590-593.

Bhalla, U. S. and R. Iyengar. 1999. Emergent properties of networks of biological signaling pathways. Science 283:381-387.

Bortolin, M. L. and T. Kiss. 1998. Human U19 intron-encoded snoRNA is processed from a long primary transcript that possesses little potential for protein coding. RNA 4: 445-454.

Boutros, M. and M. Mlodzik. 1999. Dishevelled: at the crossroads of divergent intracellular signaling pathways. Mech. Dev. 83: 27-37.

Bridgeman, B. 1995. A review of the role of efference copy in sensory and oculomotor control systems. Ann. Biomed. Eng. 23: 409-422.

Brockdorff, N. 1998. The role of Xist in X-inactivation. Curr. Opin. Genet. Dev. 8: 328-333.

Caffarelli, E., L. Maggi, A. Fatica, J. Jiricny and I. Bozzoni. 1997. A novel Mn⁺⁺-dependent ribonuclease that functions in U16 SnoRNA processing in X. laevis. Biochem. Biophys. Res. Commun. 233: 514-517.

Castelli-Gair, J., J. Muller and M. Bienz. 1992a. Function of an Ultrabithorax minigene in imaginal cells. Development 114: 877-886.

Castelli-Gair, J. E., M. P. Capdevila, J. L. Micol and A. Garcia-Bellido. 1992b. Positive and negative cis-regulatory elements in the bithoraxoid region of the Drosophila Ultrabithorax gene. Mol. Gen. Genet. 234: 177-184.

Castelli-Gair, J. E. and A. Garcia-Bellido. 1990. Interactions of Polycomb and trithorax with cis regulatory regions of Ultrabithorax during the development of Drosophila melanogaster. EMBO J. 9: 4267-4275.

Castelli-Gair, J. E., J. L. Micol and A. Garcia-Bellido. 1990. Transvection in the Drosophila Ultrabithorax gene: a Cbx1 mutant allele induces ectopic expression of a normal allele in trans. Genetics 126: 177-184.

Cavaille, J., K. Buiting, M. Kiefmann, M. Lalande, C. I. Brannan, B. Horsthemke, J. P. Bachellerie, J. Brosius and A. Huttenhofer. 2000. Identification of brain-specific and imprinted small nucleolar RNA genes exhibiting an unusual genomic organization. Proc. Natl. Acad. Sci. USA 97: 14311-14316.

Cavalier-Smith, T. 1991. Intron phylogeny: a new hypothesis. Trends Genet. 7: 145-148.

Cecconi, F., P. Mariottini and F. Amaldi. 1995. The Xenopus intron-encoded U17 snoRNA is produced by exonucleolytic processing of its precursor in oocytes. Nucleic Acids Res. 23: 4670-4676.

Chanfreau, G., G. Rotondo, P. Legrain and A. Jacquier. 1998. Processing of a dicistronic small nucleolar RNA precursor by the RNA endonuclease Rnt1. EMBO J. 17: 3726-3737.

Chervitz, S. A., L. Aravind, G. Sherlock et al. (13 co-authors). 1998. Comparison of the complete protein sets of worm and yeast: orthology and divergence. Science 282: 2022-2028.

Chinnaiyan, A. M. 1999. The apoptosome: heart and soul of the cell death machine. Neoplasia 1: 5-15.

Cho, G. and R. F. Doolittle. 1997. Intron distribution in ancient paralogs supports random insertion and not random loss. J. Mol. Evol. 44: 573-584.

Coffey, E. T., V. Hongisto, M. Dickens, R. J. Davis and M. J. Courtney. 2000. Dual roles for c-Jun N-terminal kinase in developmental and stress responses in cerebellar granule neurons. J. Neurosci. 20: 7602-7613.

Consortium, I. H. G. S. 2001. Initial sequencing and analysis of the human genome. Nature 409: 860-921.

Cousineau, B., S. Lawrence, D. Smith and M. Belfort. 2000. Retrotransposition of a bacterial group II intron. Nature 404: 1018-1021.

Croft, L., S. Schandorff, F. Clark, K. Burrage, P. Arctander and J. S. Mattick. 2000. ISIS, the intron information system, reveals the high frequency of alternative splicing in the human genome. Nature Genet. 24: 340-341.

Dano, S., P. G. Sorensen and F. Hynne. 1999. Sustained oscillations in living cells. Nature 402: 320-322.

Daugas, E., D. Nochy, L. Ravagnan, M. Loeffler, S. A. Susin, N. Zamzami and G. Kroemer. 2000. Apoptosis-inducing factor (AIF): a ubiquitous mitochondrial oxidoreductase involved in apoptosis. FEBS Lett. 476: 118-123.

Davidson, E. H., W. H. Klein and R. J. Britten. 1977. Sequence organization in animal DNA and a speculation on hnRNA as a coordinate regulatory transcript. Dev. Biol. 55: 69-84.

Delihas, N. 1995. Regulation of gene expression by trans-encoded antisense RNAs. Mol. Microbiol. 15: 411-414.

Dernburg, A. F., J. Zalevsky, M. P. Colaiacovo and A. M. Villeneuve. 2000. Transgene-mediated cosuppression in the C. elegans germ line. Genes Dev. 14: 1578-1583.

Deutsch, M. and M. Long. 1999. Intron-exon structures of eukaryotic model organisms. Nucleic Acids Res. 27: 3219-3228.

Dover, G. A. and D. Tautz. 1986. Conservation and divergence in multigene families: alternatives to selection and drift. Philos. Trans. R. Soc. Lond. B. Biol. Sci. 312: 275-289.

Duboule, D. and A. S. Wilkins. 1998. The evolution of ‘bricolage’. Trends Genet. 14: 54-59.

Duncan, I. 1987. The bithorax complex. Annu. Rev. Genet. 21: 285-319.

Eddy, S. R. 1999. Noncoding RNA genes. Curr. Opin. Genet. Dev. 9: 695-699.

Eickbush, T. H. 2000. Molecular biology: Introns gain ground. Nature 404: 940-941.

Elgar, G. 1996. Quality not quantity: the pufferfish genome. Hum. Mol. Genet. 5: 1437-1442.

Elman, J. L. 1998. Connectionism, artificial life, and dynamical systems: new approaches to old questions. In W. Bechtel and G. Graham, eds. A Companion to Cognitive Science. Basil Blackwood.

Elowitz, M. B. and S. Leibler. 2000. A synthetic oscillatory network of transcriptional regulators. Nature 403: 335-338.

Erdmann, V. A., M. Szymanski, A. Hochberg, N. de Groot and J. Barciszewski. 1999. Collection of mRNA-like non-coding RNAs. Nucleic Acids Res. 27: 192-195.

Feinbaum, R. and V. Ambros. 1999. The timing of lin-4 RNA accumulation controls the timing of postembryonic developmental events in Caenorhabditis elegans. Dev. Biol. 210: 87-95.

Ferat, J. L. and F. Michel. 1993. Group II self-splicing introns in bacteria. Nature 364: 358-361.

Filipowicz, W. 2000. Imprinted expression of small nucleolar RNAs in brain: Time for RNomics. Proc. Natl. Acad. Sci. USA 97: 14035-14037.

Filipowicz, W., P. Pelczar, V. Pogacic and F. Dragon. 1999. Structure and biogenesis of small nucleolar RNAs acting as guides for ribosomal RNA modification. Acta. Biochim. Pol. 46: 377-389.

Gardner, T. S., C. R. Cantor and J. J. Collins. 2000. Construction of a genetic toggle switch in Escherichia coli. Nature 403: 339-342.

Gemkow, M. J., P. J. Verveer and D. J. Arndt-Jovin. 1998. Homologous association of the Bithorax-Complex during embryogenesis: consequences for transvection in Drosophila melanogaster. Development 125: 4541-4552.

Geyer, P. K., M. M. Green and V. G. Corces. 1990. Tissue-specific transcriptional enhancers may act in trans on the gene located in the homologous chromosome: the molecular basis of transvection in Drosophila. EMBO J. 9: 2247-2256.

Goldsborough, A. S. and T. B. Kornberg. 1996. Reduction of transcription by homologue asynapsis in Drosophila imaginal discs. Nature 381: 807-810.

Haase, S. B. and S. I. Reed. 1999. Evidence that a free-running oscillator drives G1 events in the budding yeast cell cycle. Nature 401: 394-397.

Hastings, M. L., H. A. Ingle, M. A. Lazar and S. H. Munroe. 2000. Post-transcriptional regulation of thyroid hormone receptor expression by cis-acting sequences and a naturally occurring antisense RNA. J. Biol. Chem. 275: 11507-11513.

Hartwell, L. H., J. J. Hopfield, S. Leibler and A. W. Murray. 1999. From molecular to modular cell biology. Nature 402: C47-52.

Hasty, J., J. Pradines, M. Dolnik and J. J. Collins. 2000. Noise-based switches and amplifiers for gene expression. Proc. Natl. Acad. Sci. USA 97: 2075-2080.

Hendrickson, J. E. and S. Sakonju. 1995. Cis and trans interactions between the iab regulatory regions and abdominal-A and abdominal-B in Drosophila melanogaster. Genetics 139: 835-848.

Herbert, A. and A. Rich. 1999a. RNA processing and the evolution of eukaryotes. Nature Genet. 21: 265-269.

Herbert, A. and A. Rich. 1999b. RNA processing in evolution: The logic of soft-wired genomes. Ann. N.Y. Acad. Sci. 870:119-132.

Hermann, T. and Westhof, E. 1999. Non-Watson-Crick base pairs in RNA-protein recognition. Chem. Biol. 6: R335-43.

Hoeflich, K. P., J. Luo, E. A. Rubie, M. S. Tsao, O. Jin and J. R. Woodgett. 2000. Requirement for glycogen synthase kinase-3β in cell survival and NF-kappaB activation. Nature 406: 86-90.

Hogness, D. S., H. D. Lipshitz, P. A. Beachy, D. A. Peattie, R. B. Saint, M. Goldschmidt-Clermont, P. J. Harte, E. R. Gavis and S. L. Helfand. 1985. Regulation and products of the Ubx domain of the bithorax complex. Cold Spring Harb. Symp. Quant. Biol. 50: 181-194.

Holland, P. W. 1999. The future of evolutionary developmental biology. Nature 402: C41-44.

Hong, Y. K., S. D. Ontiveros and W. M. Strauss. 2000. A revision of the human XIST gene organization and structural comparison with mouse Xist. Mamm. Genome 11: 220-224.

Hopmann, R., D. Duncan and I. Duncan. 1995. Transvection in the iab-5,6,7 region of the bithorax complex of Drosophila: homology independent interactions in trans. Genetics 139: 815-833.

Huang, F. 1998. Syntagms in development and evolution. Int. J. Dev. Biol. 42: 487-494.

Hunter, T. 2000a. Signaling—2000 and beyond. Cell 100: 113-127.

Hurst, L. D. and N. G. Smith. 1999. Molecular evolutionary evidence that H19 mRNA is functional. Trends Genet. 15: 134-135.

Irish, V. F., A. Martinez-Arias and M. Akam. 1989. Spatial regulation of the Antennapedia and Ultrabithorax homeotic genes during Drosophila early development. EMBO J. 8: 1527-1537.

Jan, Y. N. and L. Y. January 1993. Functional gene cassettes in development. Proc. Natl. Acad. Sci. USA 90: 8305-8307.

Jiang, Z. H. and J. Y. Wu. 1999. Alternative splicing and programmed cell death. Proc. Soc. Exp. Biol. Med. 220: 64-72.

Judd, B. H. 1988. Transvection: allelic cross talk. Cell 53: 841-843.

Kreivi, J. P. and A. I. Lamond. 1996. RNA splicing: unexpected spliceosome diversity. Curr. Biol. 6: 802-805.

Lambowitz, A. M. and M. Belfort. 1993. Introns as mobile genetic elements. Annu. Rev. Biochem. 62: 587-622.

Laney, J. D. and M. D. Biggin. 1992. zeste, a nonessential gene, potently activates Ultrabithorax transcription in the Drosophila embryo. Genes Dev. 6: 1531-1541.

Lee, J. T., L. S. Davidow and D. Warshawsky. 1999. Tsix, a gene antisense to Xist at the X-inactivation centre. Nature Genet. 21: 400-404.

Lee, R. C., R. L. Feinbaum and V. Ambros. 1993. The C. elegans heterochronic gene lin-4 encodes small RNAs with antisense complementarity to lin-14. Cell 75: 843-854.

Lehmann, A. R. 2001. The xeroderma pigmentosum group D (XPD) gene: one gene, two functions, three diseases. Genes Dev. 15: 15-23.

Lipman, D. J. 1997. Making (anti)sense of non-coding sequence conservation. Nucleic Acids Res. 25: 3580-3583.

Lipshitz, H. D., D. A. Peattie and D. S. Hogness. 1987. Novel transcripts from the Ultrabithorax domain of the bithorax complex. Genes Dev. 1: 307-322.

Loeffler, M. and G. Kroemer. 2000. The mitochondrion in cell death control: certainties and incognita. Exp. Cell Res. 256: 19-26.

Lopez, A. J. 1998. Alternative splicing of pre-mRNA: developmental consequences and mechanisms of regulation. Annu. Rev. Genet. 32: 279-305.

Martinez-Abarca, F. and N. Toro. 2000. Group II introns in the bacterial world. Mol. Microbiol 38: 917-926.

Masquida, B. and Westhof, E. 2000. On the wobble GoU and related pairs. Rna 6: 9-15

Mattick, J. S. 1994. Introns: evolution and function. Curr. Opin. Genet. Dev. 4: 823-831.

Maxwell, E. S. and M. J. Fournier. 1995. The small nucleolar RNAs. Annu. Rev. Biochem. 64: 897-934.

McAdams, H. H. and A. Arkin. 1997. Stochastic mechanisms in gene expression. Proc. Natl. Acad. Sci. USA 94: 814-819.

McAdams, H. H. and L. Shapiro. 1995. Circuit simulation of genetic networks. Science 269: 650-656.

McClelland, J. L. and D. C. Plaut. 1993. Computational approaches to cognition: top-down approaches. Curr. Opin. Neurobiol. 3: 209-216.

McClelland, J. L. and D. E. Rumelhart. 1985. Distributed memory and the representation of general and specific information. J. Exp. Psychol. Gen. 114:159-197.

Mendoza, L. and E. R. Alvarez-Buylla. 1998. Dynamics of the genetic regulatory network for Arabidopsis thaliana flower morphogenesis. J. Theor. Biol. 193: 307-319.

Mestl, T., E. Plahte and S. W. Omholt. 1995. A mathematical framework for describing and analysing gene regulatory networks. J. Theor. Biol. 176: 291-300.

Mette, M. F., W. Aufsatz, J. van Der Winden, M. A. Matzke and A. J. Matzke. 2000. Transcriptional silencing and promoter methylation triggered by double-stranded RNA. EMBO J. 19: 5194-5201.

Micol, J. L., J. E. Castelli-Gair and A. Garcia-Bellido. 1990. Genetic analysis of transvection effects involving cis-regulatory elements of the Drosophila Ultrabithorax gene. Genetics 126: 365-373.

Mitchell, P., E. Petfalski, A. Shevchenko, M. Mann and D. Tollervey. 1997. The exosome: a conserved eukaryotic RNA processing complex containing multiple 3′→5′ exoribonucleases. Cell 91: 457-466.

Mitchell, P. and D. Tollervey. 2000. Musing on the structural organization of the exosome complex. Nature Struct. Biol. 7: 843-846.

Nashimoto, M. 2000. Anomalous RNA substrates for mammalian tRNA 3′ processing endoribonuclease. FEBS Lett. 472: 179-186.

Nemes, J. P., K. A. Benzow and M. D. Koob. 2000. The SCA8 transcript is an antisense RNA to a brain-specific transcript encoding a novel actin-binding protein (KLHL1). Hum. Mol. Genet. 9: 1543-1551.

Newman, A. J. 1994. Pre-mRNA splicing. Curr. Opin. Genet. Dev. 4: 298-304.

Nicoloso, M., L. H. Qu, B. Michot and J. P. Bachellerie. 1996. Intron-encoded, antisense small nucleolar RNAs: the characterization of nine novel species points to their direct role as guides for the 2′-O-ribose methylation of rRNAs. J. Mol. Biol. 260: 178-195.

Niehrs, C. and N. Pollet. 1999. Synexpression groups in eukaryotes. Nature 402: 483-487.

O'Brien, S. P., K. Seipel, Q. G. Medley, R. Bronson, R. Segal and M. Streuli. 2000. Skeletal muscle deformity and neuronal disorder in trio exchange factor-deficient mouse embryos. Proc. Natl. Acad. Sci. USA 97: 12074-12078.

Palmer, J. D. and J. M. Logsdon, Jr. 1991. The recent origins of introns. Curr. Opin. Genet. Dev. 1: 470-477.

Parrish, S., J. Fleenor, S. Xu, C. Mello and A. Fire. 2000. Functional anatomy of a dsRNA trigger. Differential requirement for the two trigger strands in RNA interference. Mol. Cell 6: 1077-1087.

Pasquinelli, A. E., B. J. Reinhart, F. Slack et al. (11 co-authors). 2000. Conservation of the sequence and temporal expression of let-7 heterochronic regulatory RNA. Nature 408: 86-89.

Pawson, T. 1995. Protein modules and signalling networks. Nature 373: 573-580.

Pelczar, P. and W. Filipowicz. 1998. The host gene for intronic U17 small nucleolar RNAs in mammals has no protein-coding potential and is a member of the 5′-terminal oligopyrimidine gene family. Mol. Cell Biol. 18: 4509-4518.

Pirrotta, V. 1990. Transvection and long-distance gene regulation. Bioessays 12: 409-414.

Pirrotta, V. 1999. Transvection and chromosomal trans-interaction effects. Biochim. Biophys. Acta 1424: M1-8.

Plunkett, K., A. Karmiloff-Smith, E. Bates, J. L. Elman and M. H. Johnson. 1997. Connectionism and developmental psychology. J. Child Psychol. Psychiatry 38: 53-80.

Potter, S. S. and W. W. Branford. 1998. Evolutionary conservation and tissue-specific processing of Hoxa 11 antisense transcripts. Mamm. Genome 9: 799-806.

Praseuth, D., Guieysse, A. L. and Helene, C. 1999. Triple helix formation and the antigene strategy for sequence-specific control of gene expression. Biochim Biophys Acta, 1489: 181-206

Prislei, S., A. Fatica, E. De Gregorio, M. Arese, P. Fragapane, E. Caffarelli, C. Presutti and I. Bozzoni. 1995. Self-cleaving motifs are found in close proximity to the sites utilized for U16 snoRNA processing. Gene 163: 221-226.

Qian, L., M. N. Vu, M. Carter and M. F. Wilkinson. 1992. A spliced intron accumulates as a lariat in the nucleus of T cells. Nucleic Acids Res. 20: 5345-5350.

Qu, L. H., A. Henras, Y. J. Lu, H. Zhou, W. X. Zhou, Y. Q. Zhu, J. Zhao, Y. Henry, M. Caizergues-Ferrer and J. P. Bachellerie. 1999. Seven novel methylation guide small nucleolar RNAs are processed from a common polycistronic transcript by Rat1p and RNase III in yeast. Mol. Cell Biol. 19: 1144-1158.

Rebane, A., R. Tamme, M. Laan, I. Pata and A. Metspalu. 1998. A novel snoRNA (U73) is encoded within the introns of the human and mouse ribosomal protein S3a genes. Gene 210: 255-263.

Reinhart, B. J., F. J. Slack, M. Basson, A. E. Pasquinelli, J. C. Bettinger, A. E. Rougvie, H. R. Horvitz and G. Ruvkun. 2000. The 21-nucleotide let-7 RNA regulates developmental timing in Caenorhabditis elegans. Nature 403: 901-906.

Roest Crollius, H., O. Jaillon, A. Bernot et al. (12 co-authors). 2000. Estimate of human gene number provided by genome-wide analysis using Tetraodon nigroviridis DNA sequence. Nature Genet. 25: 235-238.

Rubin, G. M., M. D. Yandell, J. R. Wortman et al. (55 co-authors). 2000. Comparative genomics of the eukaryotes. Science 287: 2204-2215.

Ruskin, B. and M. R. Green. 1985. An RNA processing activity that debranches RNA lariats. Science 229: 135-140.

Sanchez-Herrero, E. and M. Akam. 1989. Spatially ordered transcription of regulatory DNA in the bithorax complex of Drosophila. Development 107: 321-329.

Santoro, B., E. De Gregorio, E. Caffarelli and I. Bozzoni. 1994. RNA-protein interactions in the nuclei of Xenopus oocytes: complex formation and processing activity on the regulatory intron of ribosomal protein gene L1. Mol. Cell Biol. 14: 6975-6982.

Sharp, P. A. 2001. RNA interference-2001. Genes Dev 15: 485-490.

Shearman, L. P., S. Sriram, D. R. Weaver et al. (11 co-authors). 2000. Interacting molecular loops in the mammalian circadian clock. Science 288: 1013-1019.

Sipos, L., J. Mihaly, F. Karch, P. Schedl, J. Gausz and H. Gyurkovics. 1998. Transvection in the Drosophila Abd-B domain: extensive upstream sequences are involved in anchoring distant cis-regulatory regions to the promoter. Genetics 149: 1031-1050.

Sit, T. L., A. A. Vaewhongs and S. A. Lommel. 1998. RNA-mediated trans-activation of transcription from a viral RNA. Science 281: 829-832.

Smith, C. M. and J. A. Steitz. 1998. Classification of gas5 as a multi-small-nucleolar-RNA (snoRNA) host gene and a member of the 5′-terminal oligopyrimidine gene family reveals common features of snoRNA host genes. Mol. Cell Biol. 18: 6897-6909.

Smolen, P., D. A. Baxter and J. H. Byrne. 1999. Effects of macromolecular transport and stochastic fluctuations on dynamics of genetic regulatory systems. Am. J. Physiol. 277: C777-790.

Smolen, P., D. A. Baxter and J. H. Byrne. 2000. Modeling transcriptional control in gene networks—methods, recent results, and future directions. Bull. Math. Biol. 62: 247-292.

Sollner-Webb, B. 1993. Novel intron-encoded small nucleolar RNAs. Cell 75: 403-405.

Stoltzfus, A. 1999. On the possibility of constructive neutral evolution. J. Mol. Evol. 49: 169-181.

Stoltzfus, A., D. F. Spencer, M. Zuker, J. M. Logsdon, Jr. and W. F. Doolittle. 1994. Testing the exon theory of genes: the evidence from protein structure. Science 265: 202-207.

Szebenyi, G. and J. F. Fallon. 1999. Fibroblast growth factors as multifunctional signaling factors. Int. Rev. Cytol. 185: 45-106.

Tanaka, R., H. Satoh, M. Moriyama, K. Satoh, Y. Morishita, S. Yoshida, T. Watanabe, Y. Nakamura and S. Mori. 2000. Intronic U50 small-nucleolar-RNA (snoRNA) host gene of no protein-coding potential is mapped at the chromosome breakpoint t(3;6)(q27;q15) of human B-cell lymphoma. Genes Cells 5: 277-287.

Tarrio, R., F. Rodriguez-Trelles and F. J. Ayala. 1998. New Drosophila introns originate by duplication. Proc. Natl. Acad. Sci. USA 95: 1658-1662.

Tautz, D., M. Trick and G. A. Dover. 1986. Cryptic simplicity in DNA is a major source of genetic variation. Nature 322: 652-656.

Thieffry, D., A. M. Huerta, E. Perez-Rueda and J. Collado-Vides. 1998. From specific gene regulation to genomic networks: a global analysis of transcriptional regulation in Escherichia coli. Bioessays 20: 433-440.

Tycowski, K. T., M. D. Shu and J. A. Steitz. 1996. A mammalian gene with introns instead of exons generating stable RNA products. Nature 379: 464-466.

van der Gugten, A. A. and H. V. Westerhoff. 1997. Internal regulation of a modular system: the different faces of internal control. Biosystems 44: 79-106.

van Hoof, A., P. Lennertz and R. Parker. 2000. Three conserved members of the RNase D family have unique and overlapping functions in the processing of 5S, 5.8S, U4, U5, RNase MRP and RNase P RNAs in yeast. EMBO J. 19: 1357-1365.

van Hoof, A. and R. Parker. 1999. The exosome: a proteasome for RNA? Cell 99: 347-350.

Varani, G. and McClain, W. H. 2000. The G x U wobble base pair. A fundamental building block of RNA structure crucial to RNA function in diverse biological systems. EMBO Rep, 1: 18-23

Venter, J. C., M. D. Adams, E. W. Myers, P. W. Li, R. J. Mural, G. G. Sutton, H. O. Smith, M. Yandell et al. (274 co-authors). 2001. The sequence of the human genome. Science 291: 1304-1351.

von Neumann, J. 1982. First draft of a report on the EDVAC. In B. Randall, ed. The origins of digital computers: selected papers. Springer, Berlin.

Weng, G., U.S. Bhalla and R. Iyengar. 1999. Complexity in biological signaling systems. Science 284: 92-96.

Wightman, B., I. Ha and G. Ruvkun. 1993. Posttranscriptional regulation of the heterochronic gene lin-14 by lin-4 mediates temporal pattern formation in C. elegans. Cell 75: 855-862.

Wolf, D. M. and F. H. Eeckman. 1998. On the relationship between genomic regulatory element organization and gene regulatory dynamics. J. Theor. Biol. 195: 167-186.

Wrana, J. L. 1994. H19, a tumour suppressing RNA? Bioessays 16: 89-90.

Wu, C. T. and M. L. Goldberg. 1989. The Drosophila zeste gene and transvection. Trends Genet. 5: 189-194.

Wu, C. T. and J. R. Morris. 1999. Transvection and other homology effects. Curr. Opin. Genet. Dev. 9: 237-246.

Yang, D., H. Lu and J. W. Erickson. 2000. Evidence that processed small dsRNAs may mediate sequence-specific mRNA degradation during RNAi in drosophila embryos. Curr. Biol. 10: 1191-1200.

Yean, S. L., G. Wuenschell, J. Termini and R. J. Lin. 2000. Metal-ion coordination by U6 small nuclear RNA contributes to catalysis in the spliceosome. Nature 408: 881-884.

Yuh, C. H., H. Bolouri and E. H. Davidson. 1998. Genomic cis-regulatory logic: experimental and computational analysis of a sea urchin gene. Science 279: 1896-1902.

Zamore, P. D., T. Tuschl, P. A. Sharp and D. P. Bartel. 2000. RNAi: double-stranded RNA directs the ATP-dependent cleavage of mRNA at 21 to 23 nucleotide intervals. Cell 101: 25-33.

1. A method for identifying an eRNA or a DNA sequence comprising an eRNA-encoding sequence in the nucleome of a eukaryotic cell, said method comprising identifying non-protein-encoding nucleotide sequences within an mRNA transcript or a DNA sequence encoding same in said nucleome, determining the nucleotide sequence of said non-protein-encoding nucleotide sequence and subjecting said sequence to phenotyping to determine its effect on one or more biological events within a cell and/or determining the degree to which said sequence is conserved in the cell's genome or in the genome of other species or genera of eukaryotic cells wherein a non-protein-encoding nucleotide sequence having a biological effect in a cell or a nucleotide sequence conserved within the genome or between different cells' genomes is deemed to be an eRNA or DNA sequence comprising a nucleotide sequence encoding same.

2. A method for identifying a receiver DNA or RNA sequence, said method comprising identifying non-protein-encoding nucleotide sequences within an RNA transcript or a DNA sequence encoding same in said nucleome, determining the nucleotide sequence of said non-protein-encoding nucleotide sequence and subjecting said sequence to phenotyping to determine its effect on one or more biological events within a cell and/or determining the degree to which said sequence is conserved in the cell's genome or in the genome of other species or genera of eukaryotic cells wherein a non-protein-encoding nucleotide sequence having a biological effect in a cell or a nucleotide sequence conserved within the genome or between different cells' nucleomes is deemed to be an eRNA or DNA sequence comprising a nucleotide sequence encoding same and then contacting said eRNA with nucleome and proteome material and screening for interaction between the eRNA and an DNA or RNA or protein wherein the detection of such an interaction is indicative of a receiver molecule.

3. The method of claim 1 or 2 wherein the phenotyping comprises determining the degree to which a non-protein-encoding sequence is conserved within a cell's genome.

4. The method of claim 1 or 2 or 3 wherein the phenotyping comprises determining the degree to which a non-protein-encoding sequence is conserved amongst genomes of different species, genera or families.

5. The method of claim 1 or 2 wherein the phenotyping comprises determining a biological effect caused or associated with said non-protein-encoding sequence.

6. The method of claim 1 or 2 wherein the eRNA is or is derived from an intron.

7. The method of claim 1 or 2 wherein the eRNA is or is derived from an exon.

8. The method of claim 2 wherein the receiver DNA or RNA is located in the coding sequence of a gene or its RNA transcript, in the 3′ or 5′ flanking region of a gene or its RNA transcript, in the intron or intron-exon junction of a gene or its RNA transcript, or in an intergenic (non transcribed) region of the genome.

9. The method of claim 1 or 2 wherein the eukaryotic cell is from a vertebrate.

10. The method of claim 1 or 2 wherein the eukaryotic cell is from an invertebrate.

11. The method of claim 1 or 2 wherein the vertebrate is a mammal.

12. The method of claim 1 or 2 wherein the vertebrate is an avian species.

13. The method of claim 1 or 2 wherein the vertebrate is a reptilian species.

14. The method of claim 1 or 2 wherein the vertebrate is an amphibian species.

15. The method of claim 1 or 2 wherein the mammal is a human.

16. The method of claim 1 or 2 wherein the eukaryotic cell is from a plant.

17. The method of claim 1 or 2 wherein the plant is a monocotyledonous plant.

18. The method of claim 1 or 2 wherein the plant is a dicotyledonous plant.

19. A method for identifying a receiver protein, said method comprising identifying non-protein-encoding nucleotide sequences within an RNA transcript or a DNA sequence encoding same in said nucleome, determining the nucleotide sequence of said non-protein-encoding nucleotide sequence and subjecting said sequence to phenotyping to determine its effect on one or more biological events within a cell and/or determining the degree to which said sequence is conserved in the cell's genome or in the genome of other species or genera of eukaryotic cells wherein a non-protein-encoding nucleotide sequence having a biological effect in a cell or a nucleotide sequence conserved within the genome or between different cells' nucleomes is deemed to be an eRNA or DNA sequence comprising a nucleotide sequence encoding same and then contacting said eRNA with proteome material and screening for interaction between the eRNA and a protein wherein the detection of such an interaction is indicative of a receiver protein.

20. The method of claim 19 wherein the phenotyping comprises determining the degree to which a non-protein-encoding sequence is conserved within a cell's genome.

21. The method of claim 19 wherein the phenotyping comprises determining the degree to which a non-protein-encoding sequence is conserved amongst genomes of different species, genera or families.

22. The method of claim 19 wherein the phenotyping comprises determining a biological effect caused or associated with said non-protein-encoding sequence.

23. The method of claim 19 wherein the eRNA is an intron.

24. The method of claim 19 wherein the eRNA is an exon.

25. The method of claim 19 wherein the eukaryotic cell is from a vertebrate.

26. The method of claim 19 wherein the eukaryotic cell is from an invertebrate.

27. The method of claim 19 wherein the vertebrate is a mammal.

28. The method of claim 19 wherein the vertebrate is an avian species.

29. The method of claim 19 wherein the vertebrate is a reptilian species.

30. The method of claim 19 wherein the vertebrate is an amphibian species.

31. The method of claim 19 wherein the mammal is a human.

32. The method of claim 19 wherein the eukaryotic cell is from a plant.

33. The method of claim 19 wherein the plant is a monocotyledonous plant.

34. The method of claim 19 wherein the plant is a dicotyledonous plant.

35. A method of modulating the phenotype of a cell, said method comprising identifying an eRNA associated with the particular phenotype by the method of claim 1 or a receiver sequence for the eRNA by the method of claim 2 or 19 and manipulating the cell to up-or down-regulate the level or activity of the eRNA or its receiver sequence to thereby alter the phenotype of the cell.

36. The method of claim 35 wherein the eRNA is derived from an intron.

37. The method of claim 38 wherein the eRNA is derived from an exon.

38. The method of claim 38 wherein the receiver DNA is RNA is is located in the coding sequence of a gene or its RNA transcript, in the 3′ or 5′ flanking region of a gene or its RNA transcript, in the intron or intron-exon junction of a gene or its RNA transcript, or in an intergenic (non transcribed) region of the genome.

39. The method of claim 35 wherein the eukaryotic cell is for a vertebrate.

40. The method of claim 35 wherein the eukaryotic cell is from an invertebrate.

41. The method of claim 35 wherein the vertebrate is a mammal.

42. The method of claim 35 wherein the vertebrate is an avian species.

43. The method of claim 35 wherein the vertebrate is a reptilian species.

44. The method of claim 35 wherein the vertebrate is an amphibian species.

45. The method of claim 35 wherein the mammal is a human.

46. The method of claim 35 wherein the eukaryotic cell is from a plant.

47. The method of claim 35 wherein the plant is a monocotyledonous plant.

48. The method of claim 35 wherein the plant is a dicotyledonous plant.

49. A computer program product for assessing the likelihood of a candidate nucleotide sequence or group of nucleotide sequences being an eRNA or a receiver for an eRNA involved in network genetic signalling, said product comprising:—
(1) code that receives as input index values for one or more of features wherein said features are selected from:
(a) the transmitter sequence is derived from an intron in a protein-coding RNA transcript or an intron or an exon in a non-protein-coding RNA transcript or their DNA equivalent;
(b) the target receiver sequence lies in an intron or an exon in an RNA transcript or its DNA equivalent;
(c) the target receiver sequence lies in an intergenic genomic DNA sequence, such as a promoter region;
(d) the target sequence is a DNA or RNA sequence capable of interaction with an eRNA;
(e) the target receiver sequence lies in a 5′ untranslated region of an RNA transcript or its DNA equivalent;
(f) the target receiver sequence lies in a 3′ untranslated region of an RNA transcript or its DNA equivalent;
(g) the target receiver is a protein capable of sequence-specific recognition of an eRNA and/or its target recognition sequences;
(h) the sequence is a DNA or RNA which recognizes and/or interacts with an eRNA;
(i) the sequence comprises at least 12 nucleotides;
(j) the sequence has at least 80% nucleotide identity or complementarity to at least one sequence of the same genome or nucleome; or
(k) the sequence has at least 80% nucleotide identity or complementarity to at least one sequence in a genome or nucleome of a different species, genus or family of animal or plant cells;
(l) The sequence associates its position to a feature from available databases, for example, Genbank, the Gene Ontology databse or SWISSPORT; and
(m) The sequence associates by its position to a protein (ie. falls within the transcript) and that protein's expression profile, as determined by microarray analysis, is modulated in a specific way during a phenomona of interest, for example, highly up or down regulated in the initial phase of meiosis.
(2) code that adds said index values to provide a sum corresponding to a predictive value for said candidate sequences; and
(3) a computer readable medium that stores the codes.

50. A computer program product for assessing the likelihood of a candidate nucleotide sequence or group of nucleotide sequences being a receiver molecule involved in network signalling via an eRNA, said product comprising:—
(1) code that receives as input index values for one or more of features wherein said features are selected from:—
(a) the target receiver sequence lies in an intergenic genomic DNA sequence, such as a promoter region;
(b) the target receiver is a DNA or RNA sequence capable of interaction with an eRNA;
(c) the target receiver sequence lies in a 5′ untranslated region of an RNA transcript or its DNA equivalent;
(d) the target receiver sequence lies in a 3′ untranslated region of an RNA transcript or its DNA equivalent;
(e) the target receiver is a protein capable of sequence-specific recognition of an eRNA and/or its target recognition sequences;
(f) the sequence is a DNA or RNA which recognizes and/or interacts with an eRNA;
(g) the sequence comprises at least 12 nucleotides;
(h) the sequence has at least 80% nucleotide identity or complementarity to at least one sequence of the same genome or nucleome;
(i) the sequence has at least 80% nucleotide identity or complementarity to at least one sequence in a genome or nucleome of a different species, genus or family of animal or plant cells;
(j) The sequence associates its position to a feature from available databases, for example, Genbank, the Gene Ontology databse or SWISSPORT; and
(k) The sequence associates by its position to a protein (ie. falls within the transcript) and that protein's expression profile, as determined by microarray analysis, is modulated in a specific way during a phenomena of interest, for example, highly up or down regulated in the initial phase of meiosis.
(2) code that adds said index values to provide a sum corresponding to a predictive value for said candidate sequences; and
(3) a computer readable medium that stores the codes.

51. A computer system for assessing the likelihood of a candidate sequence or group of candidate sequences being an eRNA involved in network genetic signalling wherein said computer system comprises:—
(1) a machine-readable data storage medium comprising a data storage material encoded with machine-readable data, wherein said machine-readable data comprise index values for one or more features, wherein said features are selected from:—
(a) the transmitter eRNA sequence is derived from an intron in a protein-coding RNA transcript or an intron or an exon in a non-protein-coding RNA transcript, or their DNA equivalent;
(b) the transmitter sequence comprises at least 12 nucleotides;
(c) the transmitter sequence has at least 80% nucleotide identity or complementarity to at least one sequence of the same genome or nucleome;
(d) the transmitter sequence has at least 80% nucleotide identity or complementarity to at least one sequence in a genome or nucleome of a different species, genus or family of animal or plant cells;
(e) the transmitter sequence comprises a secondary or tertiary structure having an activity; and
(f) the transmitter sequence exhibits catalytic activity;
(2) a working memory for storing instructions for processing said machine-readable data;
(3) a central-processing unit coupled to said working memory and to said machine-readable data storage medium, for processing said machine readable data to provide a sum of said index values corresponding to a predictive value for said candidate sequences; and
(4) an output hardware coupled to said central processing unit for receiving said predictive value.

52. A computer system for assessing the likelihood of a candidate sequence or group of candidate sequences being a receiver RNA, DNA or protein involved in network gentic signalling wherein said computer system comprises:—
(1) a machine-readable data storage medium comprising a data storage material encoded with machine-readable data, wherein said machine-readable data comprise index values for one or more features, wherein said features are selected from:—
(a) the receiver sequence is located in an intron or an exon in an RNA transcript or its DNA equivalent;
(b) the receiver sequence lies in an intergenic genomic DNA sequence, such as a promoter region;
(c) the receiver sequence is located in a 5′ untranslated region of an RNA transcript or its DNA equivalent;
(d) the receiver sequence is located in a 3′ untranslated region of an RNA transcript or its DNA equivalent;
(e) the receiver sequence is a protein capable of sequence-specific recognition of an eRNA and/or its target recognition sequence;
(f) the receiver sequence is an RNA or DNA which recognizes and/or interacts with an eRNA;
(g) the receiver sequence comprises at least 12 nucleotides;
(h) the receiver sequence has at least 80% nucleotide identity or complementarity to at least one sequence of the same genome or nucleome;
(i) the receiver sequence has at least 80% nucleotide identity or complementarity to at least one sequence in a genome or nucleome of a different species, genus or family of animal or plant cells;
(j) the receiver sequence comprises a secondary or tertiary structure having an activity; and
(k) the receiver sequence exhibits catalytic activity;
(2) a working memory for storing instructions for processing said machine-readable data;
(3) a central-processing unit coupled to said working memory and to said machine-readable dvata storage medium, for processing said machine readable data to provide a sum of said index values corresponding to a predictive value for said candidate sequences; and
(4) an output hardware coupled to said central processing unit for receiving said predictive value.

53. An eRNA molecule identified by the method comprising identifying non-protein-encoding nucleotide sequences within an RNA transcript or a DNA sequence encoding same in said nucleome, determining the nucleotide sequence of said non-protein-encoding nucleotide sequence and subjecting said sequence to phenotyping to determine its effect on one or more biological events within a cell and/or determining the degree to which said sequence is conserved in the cell's genome or in the genome of other species or genera of eukaryotic cells wherein a non-protein-encoding nucleotide sequence having a biological effect in a cell or a nucleotide sequence conserved within the genome or between different cells' nucleomes is deemed to be an eRNA or DNA sequence comprising a nucleotide sequence encoding same.

54. A receiver DNA or RNA identified by the method comprising identifying non-protein-encoding nucleotide sequences within an RNA transcript or a DNA sequence encoding same in said nucleome, determining the nucleotide sequence of said non-protein-encoding nucleotide sequence and subjecting said sequence to phenotyping to determine its effect on one or more biological events within a cell and/or determining the degree to which said sequence is conserved in the cell's genome or in the genome of other species or genera of eukaryotic cells wherein a non-protein-encoding nucleotide sequence having a biological effect in a cell or a nucleotide sequence conserved within the genome or between different cells' nucleomes is deemed to be an eRNA or DNA sequence comprising a nucleotide sequence encoding same and then contacting said eRNA with nucleome material and screening for interaction between the eRNA and a DNA, RNA or protein wherein the detection of such interaction is indicative of a receiver molecule.

55. A receiver protein identified by the method comprising identifying non-protein-encoding nucleotide sequences within an RNA transcript or a DNA sequence encoding same in said nucleome, determining the nucleotide sequence of said non-protein-encoding nucleotide sequence and subjecting said sequence to phenotyping to determine its effect on one or more biological events within a cell and/or determining the degree to which said sequence is conserved in the cell's genome or in the genome of other species or genera of eukaryotic cells wherein a non-protein-encoding nucleotide sequence having a biological effect in a cell or a nucleotide sequence conserved within the genome or between different cells' nucleomes is deemed to be an eRNA or DNA sequence comprising a nucleotide sequence encoding same and then contacting said eRNA with proteome material and screening for interaction between the eRNA and a protein wherein the detection of such interaction is indicative of a receiver protein.

56. A method of inducing post transcriptional gene silencing (PTGS) in a eukaryotic cell, said method comprising identifying an eRNA having a receiver sequence in a target gene to be silenced and expressing a DNA comprising said eRNA in said cell for a time and under conditions sufficient for the target gene to be silenced.

57. The method of claim 56 wherein the cell is a plant cell.

58. The method of claim 56 wherein the cell is a mammalian cell.

59. The method of claim 58 wherein the mammalian cell is a human cell.

60. Use of an eRNA or an analog or homolog to modify a genetic network in a cell to thereby alter a cell's phenotype.

61. A method for detecting an altered genetic network said method comprising screening for the presence or absence of an eRNA or an altered level of eRNA wherein an alteration in the presence, absence or level of eRNA is indicative of an altered genetic network and thereby an altered phenotype.
